(19) United States
(12) Patent Application Publication
(10) Pub. No.: US 2023/0386610 A1
(43) Pub. Date: Nov. 30, 2023

(54) NATURAL LANGUAGE PROCESSING TO PREDICT PROPERTIES OF PROTEINS

(71) Applicant: GLAXOSMITHKLINE BIOLOGICALS SA, Rixensart (BE)

(72) Inventors: Gurpreet SINGH, Princeton, NJ (US); Ahmed ESSAGHIR, Rixensart (BE); Paul SMYTH, Rixensart (BE); Matthias BAL, Ghent (BE); Nanda Kumar SATHIYAMOORTHY, Mumbai (IN)

(73) Assignee: GLAXOSMITHKLINE BIOLOGICALS SA, Rixensart (BE)

(21) Appl. No.: 18/321,044

(22) Filed: May 22, 2023

Related U.S. Application Data
(60) Provisional application No. 63/345,128, filed on May 24, 2022.

(51) Int. Cl. G16B 40/00; G16B 45/00; G16B 20/00; G16B 30/00

(52) U.S. Cl. CPC G16B 40/00; G16B 30/00; G16B 20/00; G16B 45/00

(57) ABSTRACT

A protein language natural language processing (NLP) system is trained to predict binding affinity. Amino acids of proteins are tokenized and masked. A first neural network is trained on TCR sequences and epitope sequences in an unsupervised or self-supervised manner. The information obtained from the first phase of training is applied in a subsequent training operation via transfer learning, to a second neural network. An annotated compact dataset is used to fine-tune the second neural network in a second phase of training, and in a supervised manner, to predict biophysiochemical properties of proteins, including TCR-epitope binding.

### FIELD OF THE INVENTION

The present application relates to predicting properties of proteins using natural language processing, and more specifically, to methods, systems and computer-readable media for utilizing natural language processing to predict a binding affinity and/or a level of binding affinity between a TCR and an epitope.

### BACKGROUND

Various in silico and in vitro approaches have been developed to analyze the structural and functional features of proteins. In vitro approaches aim to understand protein structure and function using experimental techniques. For example, proteins may be synthesized, crystallized, and analyzed based on their crystal structure or characterized with various binding assays, expression assays, motility assays, luminescence assays or mechanical assays. However, such wet-lab based approaches are costly and time consuming.

De novo in silico approaches attempt to predict the second, third, and even fourth dimensional structures and corresponding functions of a protein from its primary amino acid structure by simulation, for example, using molecular dynamics simulations. However, such approaches typically have a high computational cost, are time consuming, and may not generate structures that correspond well with known biological structures. While improvements in computing architecture such as distributed computing/massively parallel super computing have increased computational power, these approaches are still costly and time-consuming to build. Further, computing resources may be shared among multiple research and scientific groups which may limit access.

More recently, machine learning driven in-silico approaches have emerged, offering alternatives to traditional time-consuming de novo computational approaches while being far less cost prohibitive than wet-lab approaches. However, there are disadvantages to machine learning techniques. Machine learning approaches may be tailored to one application with limited or no transferability to other applications, may require a large amount of expert-annotated data for a particular task, and may rely on time-consuming, trial-and-error processes of feature design and parameter selection. Thus, early generation machine learning approaches replaced time consuming and costly wet-lab work with time-consuming, expensive, trial-and-error based computational techniques, trained to analyze a single biological topic.

Recent advances in natural language processing (NLP) have led to alternatives to generating a large expert-annotated dataset. By applying self-supervised learning to train an NLP model on a repository of English text documents that adheres to a grammatical standard, the NLP system learns the lexicography of a particular language without a large, expert-annotated training dataset. In aspects, the trained NLP model may be subsequently retrained for another language task such as question answering (see, Devlin et al., BERT: Pre-training of Deep Bidirectional Transformers for Language Understanding (2019), arxiv.org/pdf/1810.04805.pdf). In other aspects, NLP approaches have been utilized for binding predictions (see, Filipavicius et al., “Pre-training Protein Language Models with Label-Agnostic Binding Pairs Enhances Performance in Downstream Tasks” (2020), https://arxiv.org/abs/2012.03084). While such approaches have worked well for natural languages having defined grammatical rules relating to sentence structure and parts of speech, the applicability of such approaches in other areas is not well understood.

The interactions of T-cell receptors (TCRs) with their cognate epitopes lie at the heart of the adaptive immune system's response¹. TCRs on the surface of a T-cell recognize immunogenic peptides, or epitopes, presented by an HLA molecule on the surface of antigen presenting cells and infected cells. The recognition and interaction of epitopes with TCRs is constrained by the HLA types and the V(D)J gene segment recombination coding for the TCR sequences^{3,4}. HLA class I molecules typically present 8-10 amino acid peptides, which then bind to TCRs within a minimum range of affinity. These peptides are generated through proteasomal cleavage of intra-cellular proteins and are recognized by CD8 T-cells. HLA class II molecules present larger peptides generated from endocytosed proteins, with variable length exceeding 14 amino-acids. HLA class II-presented peptides are recognized by CD4 T-cells⁴. TCR-epitope recognition is complex, not only because of the spectrum of physicochemical interactions between the TCR and the HLA-peptide complex, but also due to cross-reactivity: each TCR can recognize many epitopes and each epitope can be recognized by many TCRs^{4,5}. This cross-reactivity enables the emergence of public TCRs, i.e., TCRs shared by multiple individuals and immunodominant epitopes, i.e., recognized by distinct TCRs from distinct individuals^{6,7}.

TCRs are protein complexes formed by two chains: an α chain and a β chain encoded by the TRA and TRB genes, respectively. The complementarity-determining regions (CDR) of both α and β chains are the most variable regions of the TCR and the main interactors with the HLA-epitope complexes. CDRs drive T-cell specificity towards an antigen⁸. In particular, the CDR3β region of a TCR essentially accounts for the contacts with the epitope^{9,10}.

To identify T-cell epitopes, immunoinformatic tools predict the affinity of peptides for HLA molecules given an HLA allele, with output scores reflecting the strength of the peptide-HLA binding. TCR binding is not considered in such models, even though it may aid in targeting immunogenic peptides and thus reduce false positive predictions¹¹. Several attempts have been made to tackle antigen-specificity prediction from the TCR side. Emerson et. al. showed that TCR sequences were able to distinguish cytomegalovirus positive and negative patients by training a classifier based on the public TCR frequencies¹². The GLIPH and ALICE algorithms showed that TCRs contain motifs that would capture antigen recognition^{8,13}. Later works developed TCR binding classifier models, specific to each epitope. TCRex uses random forest-based classifiers¹⁴, while TCRGP uses Gaussian processes classifiers¹⁵.

More recently, deep learning-based modeling approaches have also been proposed. NetTCR implemented a convolutional neural network (CNN) architecture to predict epitope binding for any new CDR3β sequence among a list of known epitopes restricted to HLA-A*02:01¹⁶. In another work, ERGO (pEptide TCR matchinG predictiOn) implemented autoencoders and long short-term memory (LSTM) based model architecture to output binding probabilities of TCR-peptide pairs¹⁷. T-cellMatch implemented a range of natural language processing (NLP) architectures (including gated recurrent units (GRUs), LSTM, self-attention) to achieve TCR-epitope binding predictions with integration of multi-omics data generated by single-cell studies on T-cells¹⁸. Examples of language models include BERT models and Hopfield networks for immune repertoire classification^{30,31}.

An open question has been whether such models perform well on previously unseen epitopes. This has been tested by several authors from the above-mentioned papers and by ImRex models¹⁹. However, there is still a lack of epitope space representability, which would allow for better generalization performances, and limited transparency into underlying amino-acid interactions between the TCR and the epitope.

There is an ongoing need for computational approaches having the capability to accurately predict binding affinities between TCRs and epitopes (e.g., unseen epitopes) that can be performed within a suitable timeframe with accessible computing resources and with increased accuracy. There is an ongoing need for computational approaches that can perform well on previously unseen epitopes and provide transparency at the amino acid level of interactions between the TCR and the epitope.

### SUMMARY

The following paragraphs provide a summary of various aspects of training and using a natural language processing (NLP) system to predict a binding affinity and/or a level of binding affinity between a TCR and an epitope. This summary is not to be limited to the following exemplary embodiments.

### Training the NLP System

In an embodiment, a computer-implemented method for training a protein language NLP system to predict a binding affinity and/or a level of binding affinity between a TCR and an epitope is provided comprising:
in a first phase, training using one or more processors the protein language NLP system with a TCR sequence dataset and/or an epitope sequence dataset in a self-supervised manner; and
in a second phase, training using one or more processors the protein language NLP system, wherein the protein language NLP system comprises features from the first phase of training and generates a binding affinity or level of binding affinity prediction. Optionally, in the second phase, the protein language NLP system may be trained with an annotated tuple sequence dataset in a supervised manner, wherein the annotated protein sequence dataset comprises known TCR sequences and epitope sequences that are annotated with binding affinities (e.g., binding or not binding) or a level of binding affinity (e.g., ranges for weak, intermediate, or strong binding); the annotated tuple sequence dataset comprises pairs of TCR sequences and epitope sequences annotated with a binding affinity or a level thereof.

In an aspect, the method further comprises: training, in the first phase with one or more processors, the protein language NLP system using a TCR sequence dataset and/or an epitope sequence dataset that has undergone individual amino acid-level tokenization, n-mer level tokenization, or sub-word tokenization or any combination thereof of respective sequences.

In another aspect, the method further comprises: training, in the first phase with one or more processors, the predictive protein language NLP system using a TCR sequence dataset and/or an epitope sequence dataset, wherein about 10-20%, 12-18%, 14-16% or 15% of the amino acids in the TCR sequence dataset and/or epitope sequence datasets are masked.

In an aspect, the method further comprises: training with one or more processors the protein language NLP system, wherein the NLP system comprises a first neural network that is trained in the first phase and a second neural network comprising features (e.g., embeddings, variables, etc.) from the first neural network that is trained in the second phase.

In another aspect, the first neural network comprises at least one transformer model having at least one encoder and at least one decoder. In still another aspect, the transformer model comprises a transformer model with self-attention.

In still another aspect, the first neural network comprises a first and a second transformer model with self-attention. In an aspect, the transformer model with self-attention further comprises a robustly optimized bidirectional encoder representations from transformers approach model.

In another aspect, the second neural network comprises a perceptron or fully connected neural network.

In still another aspect, the method further comprises:
training the first neural network with a TCR sequence dataset and/or an epitope sequence dataset until each meeting a first criterion; and
storing features associated with the trained first neural network in memory. In aspects, stored features may include but are not limited to the configuration and parameters of the first neural network, including a model type, a number of layers, weights, inputs, outputs, hyperparameters, optimizer type, embeddings, concatenated representations of sequence and categorical feature embeddings, categorical variables, etc. In aspects, the stored features allow a user to reconstruct the trained first neural network or to transfer knowledge from training of the first neural network to the second neural network.

In still another aspect, the method further comprises:
obtaining a second neural network comprising features of the trained first neutral network;
and
training the second neural network comprising features of the first neural network until meeting a second criterion.

Optionally, during the second phase of training, the protein language NLP system may be trained with an annotated protein sequence dataset in a supervised manner, wherein the annotated protein sequence dataset comprises known TCR sequences and epitope sequences (“tuples”) that are each annotated with a binding affinity (e.g., binding or not binding) or a level of binding affinity (e.g., a numeric value, which may be mapped to a range for weak, intermediate, or strong binding) between pairs of TCR sequences and epitope sequences.

In still another aspect, the method further comprises: transferring the information associated with the trained first neutral network to a second neural network. In aspects, and in reference to transfer learning, a neural network may be modified by truncating one or more output layers of the first neural network, and replacing the truncated layers with one or more untrained layers. In aspects, the modified neural network is trained, until meeting a second criterion, with the annotated tuple sequence dataset. In other aspects, information may be transferred from a first neural network to a second neural network by providing embeddings, concatenated representations of sequence and categorical feature embeddings, categorical variables, or combinations thereof as input into the second neural network. In still another aspect, the second neural network may be trained to predict a binding affinity and/or a level of binding affinity between tuples of TCRs and epitopes.

In an aspect, the method further comprises: generating, for display on a display screen, information from a salience module that indicates a contribution of respective amino acids to the prediction of a binding affinity and/or a level of binding affinity between a TCR and an epitope.

In an aspect, the predictive protein language NLP system may be further trained with experimental data to validate a predicted binding affinity and/or a level of binding affinity between a candidate TCR and a candidate epitope sequence.

A computer-implemented method for predicting a binding affinity and/or a level of binding affinity between a TCR and an epitope using NLP comprising:
obtaining a protein language NLP system trained in a first phase using one or more processors on a TCR sequence dataset and an epitope sequence dataset in a self-supervised manner; and
training using one or more processors the obtained protein language NLP system to predict a binding affinity and/or a level of binding affinity between a TCR sequence and an epitope sequence; and
using the trained protein language NLP system to predict the binding affinity and/or a level of binding affinity between a candidate TCR and a candidate epitope.

Optionally, the second phase of training using one or more processors may train the obtained protein language NLP system (e.g., from the first phase of training) with an annotated tuple sequence dataset in a supervised manner, wherein the annotated sequence dataset comprises known TCR sequences and epitope sequences with known TCR sequences and epitope sequences that are annotated with binding affinities (e.g., binding or not binding) or a level of binding affinity (e.g., a numeric value that may be mapped to a range for weak, intermediate, or strong binding) between pairs of TCR sequences and epitope sequences.

### Trained NLP System

According to an embodiment of the present techniques, a computer-implemented method for predicting a binding affinity and/or a level of binding affinity between a TCR and an epitope using natural language processing (NLP) is provided comprising:
providing a trained protein language NLP system:
   trained, in a first phase with one or more processors, with a TCR sequence dataset and an epitope sequence dataset in a self-supervised manner, and
   trained, in a second phase with one or more processors, and including features from the first phase of training to generate a binding affinity or a level of binding affinity prediction;
receiving an input query, from a user interface device coupled to the trained protein language NLP system, comprising a candidate TCR sequence and a candidate epitope sequence; and
generating, by the trained protein language NLP system using one or more processors, an output comprising a prediction including a binding affinity or a level of binding affinity for the candidate TCR sequence and candidate epitope sequence.

In aspects, the output is displayed (optionally), on a display screen of a device (e.g., server device, client device), the output comprising the predicted binding affinity or a level of binding affinity for the candidate TCR and candidate epitope sequence. Optionally, during the second phase of training, the protein language NLP system may be trained with an annotated sequence dataset in a supervised manner, wherein the annotated protein sequence dataset comprises known TCR sequences and epitope sequences that are annotated with binding affinities (e.g., binding or not binding) or a level of binding affinity (e.g., a numeric value that may be mapped to a range for weak, intermediate, or strong binding) between pairs of TCR sequences and epitope sequences. Information may be transferred from a first neural network to a second neural network by any suitable technique (e.g., including providing embeddings, concatenated representations of sequence and categorical feature embeddings, categorical variables, or combinations thereof as input into the second neural network).

According to an embodiment, a computer-implemented method for predicting binding affinity or a level of binding affinity between a TCR sequence dataset and/or an epitope sequence dataset using NLP is provided comprising:
providing a protein language NLP system trained to predict a binding affinity or a level of binding affinity between a TCR sequence and an epitope sequence;
receiving an input query, from a user interface device coupled to the protein language NLP system, comprising a candidate TCR sequence and/or a candidate epitope sequence;
generating, by the protein language NLP system, an output comprising a prediction including one or more binding affinities or levels of binding affinities for the candidate TCR sequence and candidate epitope sequence. In aspects, the output is displayed (optionally), on a display screen of a device, the output comprising the binding affinity or level thereof of the candidate TCR sequence and/or candidate epitope sequence.

In an aspect, the computer-implemented method further comprises: accessing a trained protein language NLP system, the NLP system trained in the first phase by masking at least a portion of individual amino acids in the TCR sequence dataset and/or epitope sequence dataset. In aspects, about 10-20%, 12-18%, 14-16%, or 15% of individual amino acids in the TCR sequence dataset and/or epitope sequence dataset are masked.

In another aspect, the computer-implemented method further comprises: accessing a trained protein language NLP system, the NLP system trained in the first phase using a TCR sequence dataset and/or an epitope sequence dataset that has undergone individual amino acid-level tokenization, n-mer level tokenization, sub-work tokenization, or any combination thereof, of respective protein sequences.

In another aspect, the computer-implemented method further comprises: accessing a trained protein language NLP system, the NLP system trained in the first phase using a TCR dataset and/or an epitope sequence dataset, wherein about 10-20% of the amino acids in the TCR sequence dataset and/or epitope sequence dataset that has undergone individual amino acid-level tokenization are masked.

In an aspect, the computer-implemented method further comprises: accessing a trained protein language NLP system, the NLP system generated by training in a first phase a first neural network, and in a second phase a second neural network.

In another aspect, the first neural network comprises a transformer model having at least one encoder and at least one decoder. In still another aspect, the transformer model comprises a transformer model with self-attention.

In another aspect, the first neural network comprises a first transformer model and a second transformer model, each having at least one encoder and at least one decoder. In still another aspect, the first and/or second transformer model comprises a transformer model with self-attention. In another aspect, the transformer model with self-attention further comprises a robustly optimized bidirectional encoder representations from transformers approach model.

In yet another aspect, the second neural network comprises a fully connected neural network. In still another aspect, the second transformer model comprises a perceptron.

In another aspect, the computer-implemented method further comprises: receiving a plurality of candidate TCR and candidate epitope sequences generated in silico, generating a prediction of binding affinity and/or a level of binding affinity between pairs of TCR sequences and epitope sequences. In yet another aspect, the candidate TCRs sequences and epitope sequences are displayed according to a ranking of the predicted affinity and/or a level of binding affinity between TCR epitope pairs.

In aspects, the output of the trained protein language NLP system may be utilized to select hypothetical/in-silico therapeutic candidates for synthesis, for example, for experimental validation (e.g., to validate predicted binding affinity). In other aspects, the output of the trained protein language NLP system may be utilized to select a lead therapeutic candidate (e.g., based on binding affinity).

The computer-implemented method further comprises: receiving a plurality of candidate TCR and candidate epitope sequences; analyzing the candidate epitope and candidate TCR sequences; and predicting binding affinities or level of binding of pairs of candidate TCR and epitope sequences.

In yet another aspect, the computer-implemented method further comprises: providing for display on a display screen information from a salience module that indicates a contribution of respective amino acids to the prediction of a binding affinity and/or a level of binding affinity between a TCR and an epitope (e.g., candidate TCR and candidate epitope).

In still another aspect, the computer-implemented method further comprises: providing on the display screen information from a salience module that indicates a level of attention for each amino acid of the candidate TCR sequence and candidate epitope sequence.

In aspects, the computer-implemented method identifies antigens suitable for use as part of a vaccine composition. In aspects, the computer-implemented method identifies antigens that bind to TCRs that are suitable for use as part of a vaccine composition.

### Trained Executable NLP System

According to an embodiment of the present techniques, a computer-implemented method for predicting a binding affinity and/or a level of binding affinity between a TCR and an epitope using natural language processing (NLP) is provided comprising:
receiving an executable program corresponding to a trained protein language NLP system:
   trained, in a first phase, with a TCR sequence dataset and/or an epitope sequence dataset in a self-supervised manner, and
   trained, in a second phase and including features from the first phase;
loading the executable program into memory and executing with one or more processors the executable program corresponding to the trained protein language NLP system to generate a prediction for a binding affinity and/or a level of binding affinity between a candidate TCR and a candidate epitope.

Optionally, during the second phase of training, the protein language NLP system may be trained using one or more processors with an annotated sequence dataset in a supervised manner, wherein the annotated sequence dataset comprises known TCR sequences and epitope sequences that are annotated with binding affinities (e.g., binding or not binding) or a level of binding affinity (e.g., numeric values that can be mapped to a range for weak, intermediate, or strong binding) between pairs of TCR sequences and epitope sequences.

According to an embodiment of the present techniques, a computer-implemented method or system for predicting a binding affinity and/or a level of binding affinity between a TCR and an epitope using natural language processing (NLP) is provided comprising:
receiving an executable program corresponding to a trained protein language NLP system; and
loading the executable program into memory and executing with one or more processors the executable program corresponding to the trained protein language NLP system.

In an aspect, the computer-implemented method further comprises:
receiving an input query, from a user interface device coupled to the executable program, comprising a candidate TCR sequence and a candidate epitope sequence;
generating, by the executable program, an output comprising a prediction including a binding affinity and/or a level of binding affinity between the candidate TCR sequence and candidate epitope sequence; and
displaying (optionally), the output on a display screen of a device, the predicted binding affinity and/or a level of binding affinity between the candidate TCR sequence and candidate epitope sequence.

In another aspect, the computer-implemented method further comprises: receiving an executable program corresponding to the protein language NLP system, the NLP system trained in the first phase with a TCR sequence dataset and/or an epitope sequence dataset that has undergone individual amino acid-level tokenization, n-mer level tokenization, sub-word level tokenization of respective protein sequences, or any combination thereof.

In an aspect, the computer-implemented method further comprises: receiving an executable program corresponding to the protein language NLP system, the NLP system trained in the first phase by masking at least a portion of individual amino acids in the TCR sequence and/or epitope sequence datasets. In aspects, about 10-20%, 12-18%, 14-16%, or 15% of the amino acids in the TCR sequence dataset and/or the epitope dataset are masked.

In another aspect, the computer-implemented method further comprises: receiving an executable program corresponding to a protein language NLP system, the NLP system trained in the first phase using tokenized, masked TCR sequences and tokenized, masked epitope sequences, wherein about 10-20%, 12-18%, 14-16% or 15% of the amino acids in each of the datasets are masked.

In an aspect, the computer-implemented method further comprises: receiving an executable program corresponding to a protein language NLP system, the NLP system generated by training, in a first phase using one or more processors, a first neural network and in a second phase using one or more processors, a second neural network, comprising features from the first neural network.

In another aspect, the first neural network comprises at least one transformer model. In aspects, the transformer model comprises at least one encoder and at least one decoder. In still another aspect, the transformer model comprises a transformer model with self-attention.

In yet another aspect, the first neural network comprises a first transformer and a second transformer model. In aspects, the first and second transformer model each comprise at least one encoder and at least one decoder. In still another aspect, the first and second transformer model each comprise a transformer model with self-attention. In another aspect, the transformer model with self-attention further comprises a robustly optimized bidirectional encoder representations from transformers approach model.

In an aspect, the second neural network comprises a fully connected neural network or perceptron. Information may be transferred from a first neural network to a second neural network by any suitable technique (e.g., including providing embeddings, concatenated representations of sequence and categorical feature embeddings, categorical variables, or combinations thereof as inputs into the second neural network from the first neural network).

In another aspect, the computer-implemented method further comprises: receiving a plurality of candidate TCR and epitope sequences generated in silico, generating using one or more processors a prediction of a binding affinity and/or a level of binding affinity for the respective candidate TCR sequence and candidate epitope sequence. In yet another aspect, the candidate TCR sequence and candidate epitope sequence are displayed according to a ranking of the level of binding affinity and/or as groups of binders and non-binders.

In yet another aspect, the computer-implemented method further comprises: providing for display on a display screen information from a salience module that indicates a contribution of respective amino acids to the prediction of a binding affinity and/or a level of binding affinity between a TCR sequence and an epitope sequence.

In still another aspect, the computer-implemented method further comprises: providing on the display screen information from a salience module that indicates a level of attention for each amino acid of the candidate TCR sequence and candidate epitope sequence.

### System

In an embodiment, a system or apparatus is provided for training a protein language NLP system comprising one or more processors to predict a binding affinity and/or a level of binding affinity between a TCR and an epitope according to any of the methods provided herein.

A system or apparatus to predict a binding affinity and/or a level of binding affinity between a TCR and an epitope comprising one or more processors for executing instructions corresponding to a protein language NLP system to:
access a protein language NLP system trained to predict a binding affinity and/or a level of binding affinity between a TCR sequence and an epitope sequence;
receive an input query, from a user interface device coupled to the trained protein language NLP system, comprising a candidate TCR sequence and a candidate epitope sequence;
generate, by the trained protein language NLP system, a prediction including a binding affinity and/or a level of binding affinity between the candidate TCR sequence and candidate epitope sequence; and
display (optionally), on a display screen of a device, the predicted binding affinity and/or level of binding affinity between the candidate TCR sequence and candidate epitope sequence.

In aspects, a system or apparatus is provided to predict a binding affinity and/or a level of binding affinity between the candidate TCR sequence and candidate epitope sequence comprising one or more processors for executing instructions corresponding to a protein language NLP system, the system:
trained, in a first phase, with a TCR sequence dataset and/or an epitope sequence dataset in a self-supervised manner, and
trained, in a second phase and including features from the first phase to generate a prediction of a binding affinity or a level thereof. Optionally, in a second phase, the protein language NLP system may be trained with an annotated protein sequence dataset (e.g., tuples of TCR and epitope sequences) in a supervised manner, wherein the annotated protein sequence dataset comprises known TCR sequences and epitope sequences that are annotated with binding affinities (e.g., binding or not binding) or a level of binding affinity (e.g., numerical values that may be mapped to ranges for weak, intermediate, or strong binding) between pairs of TCR sequences and epitope sequences.

In an embodiment, a system or apparatus is provided for executing instructions corresponding to a protein language NLP system to predict a binding affinity and/or a level of binding affinity between the candidate TCR sequence and candidate epitope sequence according to the methods provided herein.

In another aspect, the system comprises a first neural network comprising at least one transformer model. In aspects, the transformer model comprises at least one encoder and at least one decoder. In still another aspect, the transformer model comprises a transformer model with self-attention.

In other aspects, the first neural network comprises a first transformer model and a second transformer model. In aspects, the first and second transformer model each comprise a transformer model with self-attention. In another aspect, the transformer model with self-attention further comprises a robustly optimized bidirectional encoder representations from transformers approach model. In an aspect, the first transformer may be trained on a tokenized, masked TCR sequence dataset and the second transformer may be trained on a tokenized, masked epitope sequence dataset.

In yet another aspect, the system comprises a second neural network comprising a fully connected neural network or perceptron. Information may be transferred from a first neural network to a second neural network by any suitable technique (e.g., including providing embeddings, concatenated representations of sequence and categorical feature embeddings, categorical variables, or combinations thereof as input into the second neural network).

### Computer Readable Media

According to yet another embodiment, a computer program product is provided, the computer program product comprising a computer readable storage medium having instructions for training a protein language NLP system to predict a binding affinity and/or a level of binding affinity between a TCR and an epitope embodied therewith, the instructions executable by one or more processors to cause the processors to train the protein language NLP system to predict the binding affinity and/or a level of binding affinity between the TCR sequence and the epitope sequence according to the methods provided herein.

According to still another embodiment, a computer program product for predicting a binding affinity and/or a level of binding affinity between a TCR sequence and an epitope sequence is provided, the computer program product comprising a computer readable storage medium having instructions corresponding to a protein language NLP system embodied therewith, the instructions executable by one or more processors to cause the processors to predict a binding affinity and/or a level of binding affinity between a TCR sequence and an epitope sequence according to the methods provided herein.

In another aspect, the first neural network comprises at least one transformer model. In aspects, the transformer model comprises at least one encoder and at least one decoder. In still another aspect, the transformer model comprises a transformer model with self-attention.

In yet another aspect, the first neural network comprises a first transformer model and a second transformer model. In aspects, the first and second transformer model comprise at least one encoder and at least one decoder. In still another aspect, the first and second transformer model each comprise a transformer model with self-attention. In another aspect, the first and second transformer models with attention further comprise a robustly optimized bidirectional encoder representations from transformers approach model.

In still other aspects, a computer-readable data carrier is provided having stored thereon the computer program product for predicting a binding affinity and/or a level of binding affinity according to any of the methods or systems provided herein.

In another aspect, a computer-readable storage medium is provided having stored thereon the computer program product for predicting a binding affinity and/or a level of binding affinity according to any of the methods or systems provided herein.

In other aspects, a system is provided comprising one or more processors and the computer readable storage medium/computer program product for predicting a binding affinity and/or a level of binding affinity between a TCR sequence and an epitope sequence according to any of the methods or systems provided herein. The summary is not intended to restrict the disclosure to the aforementioned embodiments. Other aspects and iterations of the disclosure are provided below.

### BRIEF DESCRIPTION OF THE DRAWINGS

Generally, like reference numerals in the various figures are utilized to designate like components.

FIG. 1 is an illustration of an example computing environment for the protein language NLP system in accordance with certain aspects of the present disclosure.

FIG. 2A is a block diagram of the protein language NLP system of FIG. 1 in accordance with certain aspects of the present disclosure.

FIG. 2B is a block diagram of the protein language NLP system pipeline of FIG. 2A in accordance with certain aspects of the present disclosure.

FIG. 2C shows an illustration of the protein language NLP pipeline according to certain embodiments herein.

FIG. 2D shows an example architecture of an embodiment of a cross attention module according to aspects provided herein.

FIG. 3A is an illustration of individual amino acid-level tokenization of a protein sequence in accordance with certain aspects of the present disclosure.

FIG. 3B is an illustration of n-mer tokenization of a protein sequence in accordance with certain aspects of the present disclosure.

FIG. 3C is an illustration of sub-word tokenization of a protein sequence in accordance with certain aspects of the present disclosure.

FIG. 4 is an illustration of randomly masking a tokenized protein sequence in accordance with certain aspects of the present disclosure.

FIG. 5A is a flow diagram showing generation of a training dataset for a first neural network in accordance with certain aspects of the present disclosure.

FIG. 5B is a flow diagram showing training of a first neural network with a first training dataset in accordance with certain aspects of the present disclosure.

FIG. 5C is a flow diagram showing training of a second neural network subjected to transfer learning with a second dataset in accordance with certain aspects of the present disclosure.

FIG. 5D is a flow diagram showing updating a trained second neural network with experimental data in accordance with certain aspects of the present disclosure.

FIG. 6 is an illustration of generating prediction probabilities for each masked instance of an amino acid by the first neural network trained on randomly masked, tokenized protein sequences in accordance with certain aspects of the present disclosure.

FIG. 7A is a high-level flowchart of operations of training a protein language NLP system, in accordance with certain aspects of the present disclosure.

FIG. 7B is another flowchart of example operations for training a protein language NLP system comprising one or more transformers that predicts a binding affinity of an epitope to a TCR, in accordance with the embodiments provided herein.

FIG. 7C is a flowchart of operations, including operations by a cross attention module, in accordance with the embodiments provided herein.

FIG. 7D is a flowchart of example operations for accessing a trained predictive protein language NLP system comprising one or more transformers that predicts a binding affinity or a level of binding of an epitope sequence to a TCR sequence, in accordance with the embodiments provided herein.

FIG. 7E is a flowchart of example operations for receiving an executable program corresponding to a trained protein language NLP system comprising one or more transformers that predict a binding affinity or a level of binding of an epitope sequence to a TCR sequence, in accordance with the embodiments provided herein.

FIG. 8 is a block diagram of an example computing device, in accordance with certain aspects of the present disclosure.

FIG. 9A is a diagrammatic illustration of a model architecture used for predicting TCR-epitope binding affinity in accordance with certain aspects of the present disclosure.

FIG. 9B shows results of predicted TCR-binding affinities for a plurality of epitopes by the protein language NLP system in accordance with certain aspects of the present disclosure.

FIG. 10A is a screenshot showing aspects of a user interface in accordance with certain aspects of the present disclosure.

FIG. 10B is another screenshot of a user interface showing an enlarged view of classification results in accordance with certain aspects of the present disclosure.

FIG. 10C is another screenshot of a user interface showing an enlarged view of a salience module in accordance with certain aspects of the present disclosure.

FIG. 10D is another screenshot showing layers of attention in accordance with certain aspects of the present disclosure.

FIG. 11 shows model performances on three different splitting strategies. Model performance was compared under three different splitting strategies, namely (1) random CDR3 assignment, (2) Ting clustered CDR3 assignment, and (3) assignment based on epitope clustering, reflecting increasing order of complexity for the generalizability of the protein language model. The protein language models were trained and tested using 5-fold cross-validation.

FIG. 12 shows protein language model performances per epitope by the number of TCRs. Each dot represents an epitope colored by its number of TCRs (CDR3β) in the ting-based training split.

FIG. 13 shows protein language model performances per epitope edit distance between train and test sets. ROC-AUC metrics in function of edit distance between epitopes in the test set were compared to the epitopes in the train set. Each dot represents an epitope, and its color represents the number of TCR instances for that epitope (the darker the color the higher the number of TCRs).

FIG. 14 shows comparison of the performance of the protein language model with Titan and ImRex models using three different splitting strategies. The performances of Titan and ImRex models have been evaluated on the exact same data splits used to train and evaluate the protein language model. Either the original trained models for inference only were used or their model architectures were fully re-trained. In the inference mode (original), the models were used as obtained from their respective repositories and were tested on the held-out data split. For the retrained comparisons, all models were trained from scratch. In the case of Titan, SMILES (SMI) or amino-acid (AA) encodings for epitope sequences were used, wherein the TCR sequences were always encoded as AA; either the models were fine-tuned using their original embeddings or the models were fully retrained (random: random assignments of TCRs to train/test splits; ting: ting based TCR cluster assignments; epitope cluster: assignments to train/test splits constrained by epitope clusters).

FIGS. 15A-15D show a Local Interpretable Model-Agnostic Explanations (LIME) for the binding between TCR and epitope sequence for PDB ID 2VLJ. FIG. 15A shows a 3D structure of the 2VLJ entry in PDB database, which shows a TCR beta (CASSRSSSYEQYF): TCR alpha complex binding GILGFVFTL epitope presented by HLA class I (HLA-A*02:01):Beta-2 microglobulin complex. FIG. 15B shows a heatmap visualizing the distance matrix (in Å units) between amino acids in TCR CDR3β to their pairs in the epitope side. Distances less than 8 Å showed strong H-bond interactions (e.g., V6 epitope-R6 and S7 CDR3β interactions showed 8 Å and 7 Å respectively). The smaller the distance, the higher the interaction. The below row (MIN) represents the minimum distance of CDR3β amino acids to each of the epitope amino acids. FIG. 27C shows a heatmap visualization showing the LIME scores as computed for each amino acid in the CDR3β chain (CASSRSSSYEQYF) when predicting its interaction with GILGVFTL epitope. The position of each amino acid in the sequence is presented in the x-axis and its identity in the y-axis. A color scale may be used to show strong or weak effects in the binding predictions. FIG. 15D shows the crystal structure of the complex which shows the interactions of epitope V6 amino-acid with CDR3β R6 and S7 amino-acids via hydrogen bond and water molecules.

FIGS. 16A-16C show a comparison of performance of the protein language NLP model with other ML models on three different splitting strategies. Here, a −gram with a stride of 1 is used.

FIGS. 17A-17C show a comparison of performance of the protein language model with other ML models on three different splitting strategies. Here we have used 2-gram with a stride of 1 for generating a one-hot-encoded version of the amino acid sequences to train other ML models.

FIGS. 18A-18C show a comparison of performance of the protein language model with other ML models on three different splitting strategies. Here, a 3-gram with a stride of 1 is used for generating a one-hot-encoded version of the amino acid sequences to train simpler ML models.

FIGS. 19A and 19B show flowcharts of data preprocessing operations according to embodiments herein. In aspects, preprocessing of the dataset leads to improved classification by standardizing amino acid sequences (e.g., capping if needed), ensuring that components of the dataset are not overweighted (e.g., removing duplicates), and identifying categories of amino acids (e.g., HLA class I) that lead to improved predictions. In aspects, optionally, preprocessing may also include randomization of TCR and MHC pairing (while preserving MHC antigen specificity) to create a negative training dataset, which also leads to an improvement in the accuracy of predictions.

### DETAILED DESCRIPTION

Amino acids are the building blocks from which a variety of macromolecules are formed, including peptides, proteins, and antibodies. These macromolecules play pivotal roles in a variety of cellular processes, for example, by forming enzyme complexes, acting as messengers for signal transduction, maintaining physical structures of cells, and regulating immunological responses. For example, enzyme complexes catalyze biochemical reactions, messengers act in various signal transduction pathways to regulate and control cellular processes, scaffold and support proteins provide shape and mechanical support to cells, and antibodies provide an immune system defense against viruses and bacteria.

Amino acids have an amino group (N-terminus), a carboxyl group (C-terminus), and an R group (a side chain) that confers various properties (e.g., polar, nonpolar, acidic, basic, etc.) to the amino acid. Amino acids may form chains through peptide bonds, which is a chemical bond formed by joining the C-terminus of an amino acid with the N-terminus of another amino acid. There are twenty naturally occurring amino acids, and unnaturally occurring amino acids have been synthesized as well. The amino acid side chains are thought to influence protein folding and shape.

The overall shape of a protein may be described at various levels, including primary, secondary, tertiary, and quaternary structures. The sequence or order of amino acids in a protein corresponds to its primary structure, also referred to as a protein backbone. Secondary structures such as alpha helices, beta sheets, turns, coils or other structures may form locally along the protein backbone. A three-dimensional shape of the protein structure/subunit, which includes local secondary structures, forms a tertiary structure, and quaternary structures are formed from the association of multiple protein structures/subunits. Thus, the structure of proteins may be described at various levels. Proteins range in size from tens to thousands of amino acids, with many proteins on the order of about 300 amino acids.

The sequence of amino acids confers structure and protein function. A protein may have one or more domains (e.g., a local three-dimensional fold relative to the full protein structure) corresponding to a particular function and such domains may be evolutionarily conserved. Thus, evolutionary demands, at least in part, are thought to influence protein sequences, leading to conservation of amino acids at positions that govern protein folding and/or function.

By self-supervised learning, it is meant that supervision is not needed, as the data itself (e.g., next amino acid) provides supervision. By supervised learning, it is meant that annotated/labeled data is provided so that the system learns to map an input to an output based on example input output pairs. The NLP models referred to herein, unless otherwise indicated, generally include a trained machine learning algorithm with one or more of embedded data, parameters, variables, configurations, and inputs/outputs related to training the machine learning algorithm. A machine learning algorithm generally refers to an untrained algorithm (e.g., procedures implemented in source code).

Natural language processing refers to a subfield of artificial intelligence geared towards processing of text-based input. Artificial neural networks may be utilized for natural language processing techniques. In the present application, NLP techniques are applied to protein sequences, including protein sequences for epitopes and TCR or fragments thereof.

Present techniques may further be applied to a wide variety of applications in vaccine development including TCR-epitope recognition, etc. These techniques may be used to prioritize vaccine antigens based upon a categorization and/or a ranking provided by the protein language NLP system. Present techniques may also be used to generate and explore binding predictions for novel TCR and epitope sequences (e.g., TCR, epitopes tuples) as well as interrogate and visualize which amino acid residues contribute to the prediction.

In aspects, the protein language NLP system includes one or more transformer models. The transformer model includes a transformer model with attention (e.g., self-attention). In aspects, the transformer model is a robustly optimized bidirectional encoder representation with transformers approach model.

Advantages of present techniques include by training a first neural network during a first phase, the system learns the lexicography of epitope and TCR sequences. The knowledge gained from training the first neural network in the first phase and enhanced with cross attention may be transferred to a second neural network and fine-tuned for further improvements in predicting binding affinities or levels of binding affinities between TCR sequences and epitopes. During the second phase, fine-tuning is performed with an annotated, compact dataset (e.g., listing known tuples of TCR and epitopes with an indication of binding affinity or a level of binding affinity) and may typically be performed more quickly than the first phase of training. These approaches provide an alternative to time consuming, one-shot machine learning approaches that need large amounts of annotated data, while offering accelerated development of machine learning applications.

In addition to offering reduced computational time as compared to other approaches, present techniques do not rely on ingesting large volumes of data that may be difficult to obtain, such as atomic data needed by molecular simulation techniques. Instead, present approaches first train on TCR and/or epitope sequences in an unsupervised/self-supervised manner to learn the features of TCR and epitope sequences, which relates to structure, and then apply this knowledge in a second phase of training to fine-tune the system to predict binding affinities or a level thereof.

Surprisingly, present approaches apply natural language processing techniques (e.g., neural nets such as transformers and transfer learning) to the biological domain, offering improved predictive capabilities of binding affinities between TCR and epitopes that meet or exceed current benchmarks. With reference now to the figures, examples of a computer-implemented method, a computer-implemented system, a computer program product and results are provided.

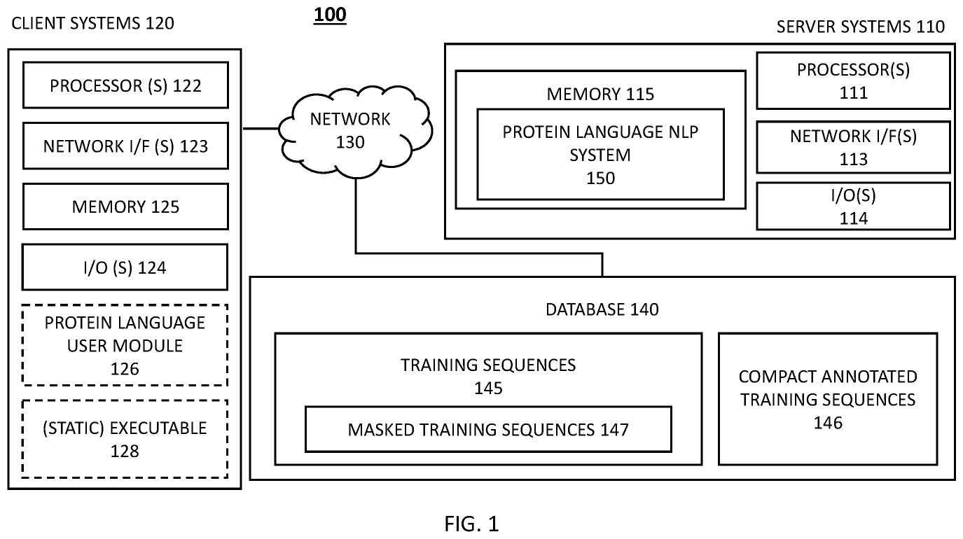

FIG. 1 shows an example computing environment 100 for use with the protein language NLP system provided herein. The computing environment may include one or more server systems 110 and one or more client/end-user systems 120. Server systems 110 may communicate remotely with client systems 120 over a network 130 with any suitable communication medium (e.g., via a wide area network (WAN), a local area network (LAN), the Internet, an Intranet, or any other suitable communication medium, hardwire, a wireless link, etc.). Server systems 110 may comprise a protein language NLP system 150, stored in memory 115 that is trained on sequences stored in database 140.

Server systems 110 may comprise a computer system equipped with one or more processor(s) 111 (e.g., CPUs, GPUs, etc.), one or more memories 115, internal or external network interfaces (I/F) 113 (e.g., including but not limited to a modem, a network card, etc.), and input/output (I/O) interface(s) 114 (e.g., a graphical user interface (GUI) or other interface (e.g., command line prompts, menu screens, etc.) to receive input from an input device (e.g., a keyboard, a mouse, etc.) or to display output on a display screen. The server system may comprise any commercially available software (e.g., server operating system, server/client communications software, browser/interface software, device drivers, etc.) as well as custom software (e.g., protein language NLP system 150, etc.). In some aspects, server systems 110 may be, for example, a server, a supercomputer, a distributed computing platform, etc. Server systems 110 may execute one or more applications, such as software for the protein language NLP system 150 that predicts binding affinities or levels thereof of tuples of candidate TCR and epitope sequence(s).

Memory 115 stores program instructions that provide the functionality for the predictive protein language NLP system 150. These program instructions are generally executed by processor(s) 111, alone or in combination with other processors.

Client systems 120 may comprise a computer system equipped with one or more processor(s) 122, one or more memories 125, internal or external network interface(s) (I/F) 123 (e.g., including but not limited to a modem, a network card, etc.), input/output (I/O) interface(s) 124 (e.g., a graphical user interface (GUI) or other interface (e.g., command line prompts, menu screens, etc.) to receive input from an input device (e.g., a keyboard, a mouse, etc.) by a user or to display output on a display screen. The client system 120 may comprise any commercially available software (e.g., operating system, server/client communications software, browser/interface software, etc.) as well as any custom software (e.g., protein language user module 126, etc.). In some aspects, client systems 120 may be, for example, any suitable computing device, such as a desktop computing device, a laptop computing device, a tablet computing device, a mobile phone computing device, etc.

Client systems 120 may execute via one or more processors one or more applications, such as software corresponding to the protein language user module 126. In some aspects, protein language user module 126 may enable a user to provide candidate epitope and TCR sequence(s) to the protein language NLP system and to receive predictions of binding affinities or levels thereof for said sequence(s). In aspects, client systems 120 may provide one or more candidate TCR and epitope sequence(s) to server systems 110 for analysis by protein language NLP system 150, and protein language NLP system may analyze the candidate sequence(s) to return one or more binding affinities or levels thereof predicted by the system. Thus, in aspects, client systems 120 may access server systems 110, which hosts/provides the trained protein language NLP system.

In aspects, the trained protein language NLP system 150 continues to undergo additional training as new data is available. In this dynamic mode of operation, the protein language NLP system continues to be trained at the server side, and a client system may access the protein language NLP system through network 130 via protein language user module 126.

In an alternative embodiment, once the protein language NLP system has been trained (e.g., with a first phase of training (unsupervised) and a second phase of training with a specific annotated dataset), the trained protein language NLP system may be converted into an executable for execution on client systems 120, allowing analysis of candidate sequence(s) to proceed in a stand-alone mode of operation.

In this stand-alone mode of operation, the client system runs an executable 128 corresponding to the trained protein language NLP system in a static mode of operation. In this mode, the static executable 128 corresponding to the protein language NLP system 150 does not undergo additional training and is locked in a static configuration. In operation, the executable may receive and analyze candidate sequence(s) to return one or more predicted binding affinities or levels thereof for the candidate sequence(s).

Typically, the client device includes protein language user module 126 or executable 128.

Thus, in aspects, protein language NLP system 150 may be compiled into an easy-to-use python package/executable. In other aspects, protein language NLP system 150 may be provided as a software as a service (“SaaS”), in which a user remotely accesses the protein language NLP system 150 (trained) hosted by a server.

The environment of present invention embodiments may include any number of computers or other processing systems (e.g., client/end-user systems, server systems, etc.) and databases or other storage repositories arranged in any suitable fashion. These embodiments are compatible with any suitable computing environment (e.g., distributed, cloud, client-server, server-farm, network, mainframe, stand-alone, etc.).

A database 140 may store various data (e.g., training sequences 145 (e.g., TCR sequences and epitope sequences) for training in phase one, and annotated, compact training sequences 146 for fine-tuning in phase two). In some aspects, masked training sequences may be stored in database 140 as masked training sequences 147. In some aspects, the database may be implemented by any conventional or other suitable database or storage system, and may be local to or remote from server systems 110 and client systems 120. In aspects, the database may be connected to a network 130 and may communicate with the client and/or the server via any appropriate local or remote communication medium (e.g., local area network (LAN), wide area network (WAN), Internet, Intranet, hardwire, wireless link, cellular, satellite, etc.).

The system may employ any number of any conventional or other databases, data stores or storage structures (e.g., files, databases, data structures, data or other repositories, etc.) to store information (e.g., training sequences, candidate sequences, binding affinity or levels thereof predictions, neural network features such as neural network configurations and/or hyperparameters, etc.). The database system may be included within or coupled to the server and/or client systems. The database systems and/or storage structures may be remote from or local to the server or other processing systems, and may store any desired data.

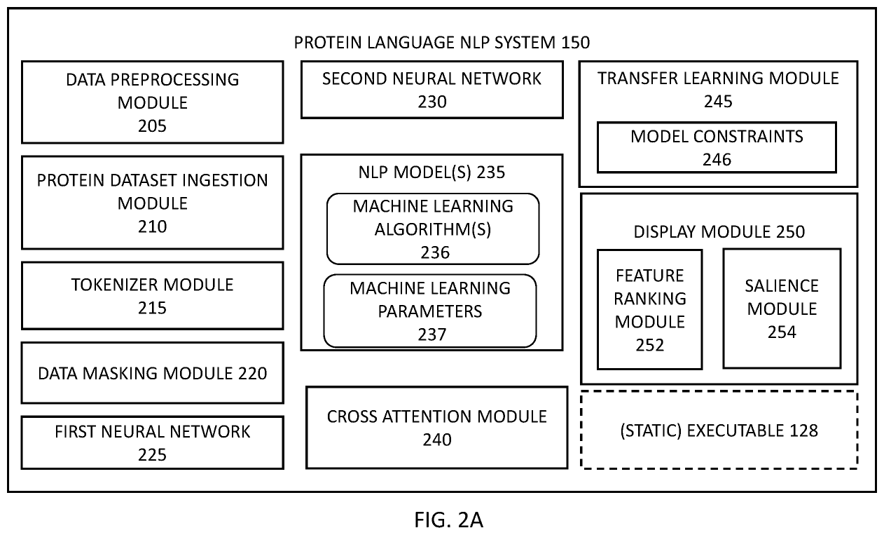

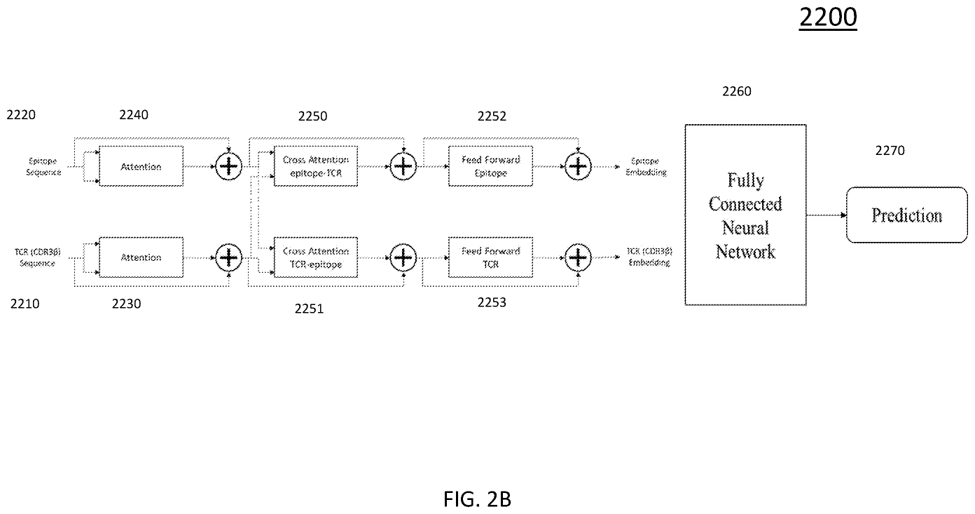

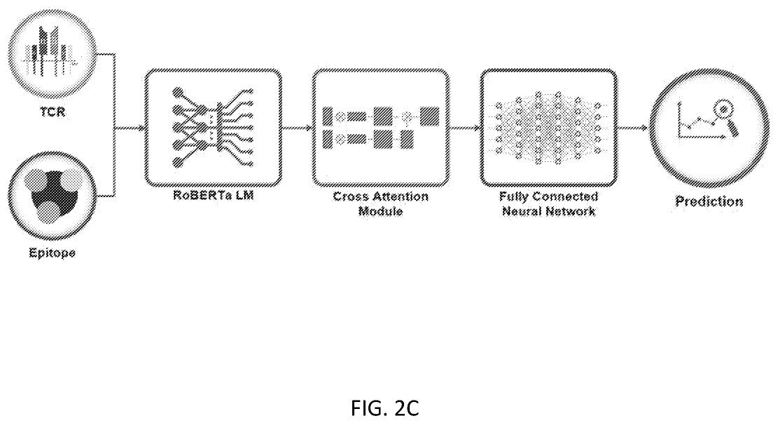

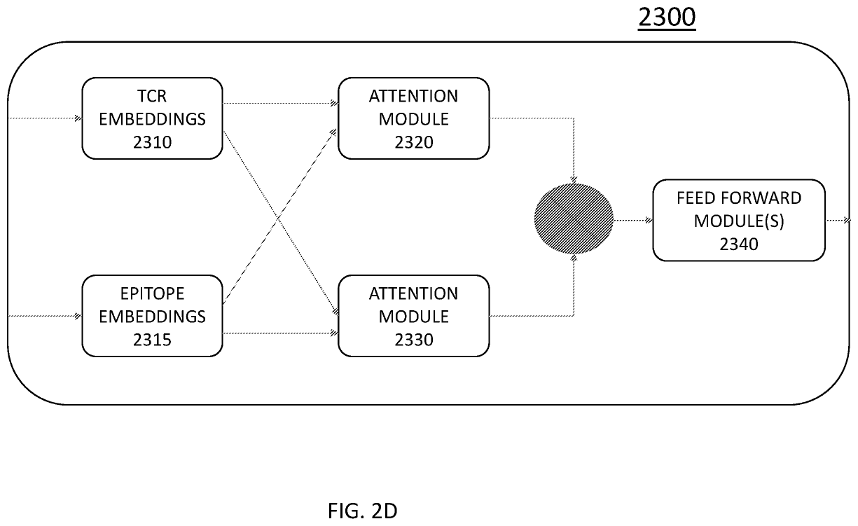

As shown in FIG. 2A, the protein language NLP system 150 comprises a data preprocessing module 205, a protein dataset ingestion module 210, a tokenizer module 215, a data masking module 220, a first neural network 225 (an artificial neural network), a second neural network 230 (an artificial neural network), NLP Models(s) 235, a cross attention module 240, a transfer learning module 245, and a display module 250. In some aspects, the protein language NLP system 150 may also include an executable 128 configured to run on client systems/devices. The executable 128, which corresponds to the protein language NLP system 150, does not undergo further learning, but rather, is compiled as a static configuration. It is to be understood that the neural networks provided herein refer to artificial neural networks.

The protein language NLP system 150 comprises a first neural network 225 and a second neural network 230 that is trained in two phases. In the first phase, the first neural network 225 is trained on datasets of TCR sequences and epitope sequences. By training on this dataset, the protein language NLP system learns, in an unsupervised/self-supervised manner, “rules” of TCR and epitope sequences. An annotated dataset is not needed in the first phase of training as the next amino acid is known (except for end of sequence). Once the first phase of training is complete, transfer learning module 245 transfers knowledge from training the first neural network to a second neural network 230. The second neural network is then fine-tuned using an annotated, compact dataset (e.g., TCR sequences and epitopes, annotated with binding affinities or a level of binding affinity thereof) that is of a reduced size as compared to the dataset(s) used in the first phase of training.

Thus, the first neural network refers to a neural network trained in a first phase, and the second neural network refers to a neural network trained in a second phase.

NLP model(s) 235 may contain any suitable machine learning algorithm(s) 236 (e.g., typically, an untrained algorithm) for the embodiments provided herein. Models may be constructed by training algorithms including but not limited to neural networks, deep learning networks, generative networks, convolutional neural networks, long short term memory networks, transformers, transformers with attention, robustly optimized neural networks, etc. In aspects, a robustly optimized bidirectional encoder representations from transformers approach (RoBERTa) is provided (see, e.g., Liu et al., RoBERTa: A Robustly Optimized BERT Pretraining Approach, (2019), arXiv.org/abs/1907.11692). Additional types of machine learning algorithms may include, for example, a classifier(s) to predict a label or category of a given input value (e.g., binding, not binding); and/or a regression model(s) to predict a discrete value (e.g., a value within a low range, an intermediate range, or a high range of affinity binding). Ranges may map discrete values to particular properties (e.g., a first range to indicate strong binding, a second range to indicate medium/intermediate binding, and a third range to indicate weak binding). Machine learning parameters 237, which comprises information from training the machine learning algorithm in the first and/or second phase, may also be stored within memory 115 or database 140.

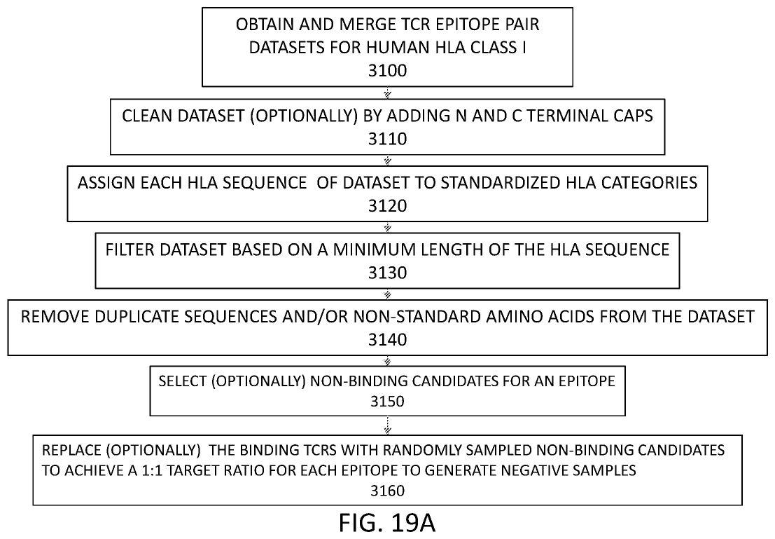

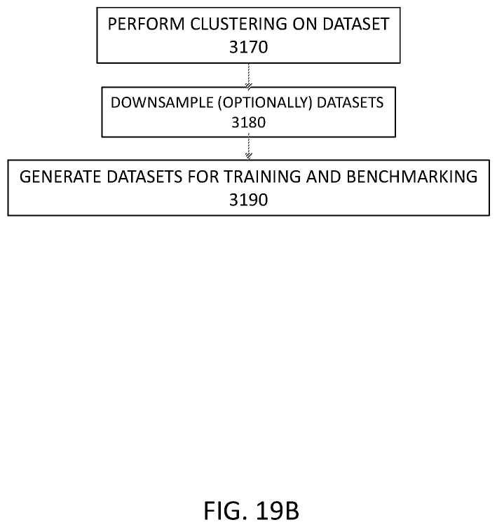

Data preprocessing module 205 processes the TCR sequence dataset and the epitope sequence datasets prior to tokenization, masking, and provision of the datasets to the protein dataset ingestion module 210. FIGS. 19A and 19B show example operations of the data preprocessing module 205. This module may select/filter for a specific type of HLA category, add N or C terminal caps (if needed) to standardize the data sets, and may cluster sequences to generate subsets of data for training. This module may also generate non-binding datasets and perform other operations to generate other types of datasets for training and benchmarking.

Protein dataset ingestion module 210 ingests datasets from public and/or private repositories. In some aspects, the protein dataset ingestion module 210 may ingest protein sequences downloaded from a database (e.g., with or without preprocessing from data preprocessing module 205), and may format the dataset (e.g., by removing extraneous text and/or extracting the protein sequences from database records, etc.) in order to provide this data as input to the tokenizer module 215. Publicly and/or privately available sequence data may be provided in any suitable format (e.g., FASTA, SAM, etc.) from any suitable source (public or private databases). In various implementations, the protein sequence may be parsed and stored in one or more data structures, including but not limited to trees, graphs, lists (e.g., linked list), arrays, matrices, vectors, and so forth.

In aspects, the protein dataset ingestion module may receive as input protein sequence information downloaded from a publicly available database, e.g., in FASTA, XML, or another text based format. For example, individual records of a bulk database download may contain one or more fields including a record identifier (e.g., a numeric and/or text identifier such as one or more NCBI identifiers including a library accession number, protein name, etc.) as well as the protein sequence itself (amino acid sequence). The protein dataset ingestion module may parse each downloaded record/entry obtained from the database (e.g., in a FASTA, XML, text, or other format etc.) into a data structure or structured data (e.g., a format such as an array or matrix) suitable for input into the tokenization module. For example, the output of the protein dataset ingestion module may comprise a data structure or structured data with each entry including an identifier and the corresponding sequence listing (e.g., an array or matrix of entries). In some aspects, the protein dataset module 210 may receive data pre-processed according to FIGS. 19A and 19B.

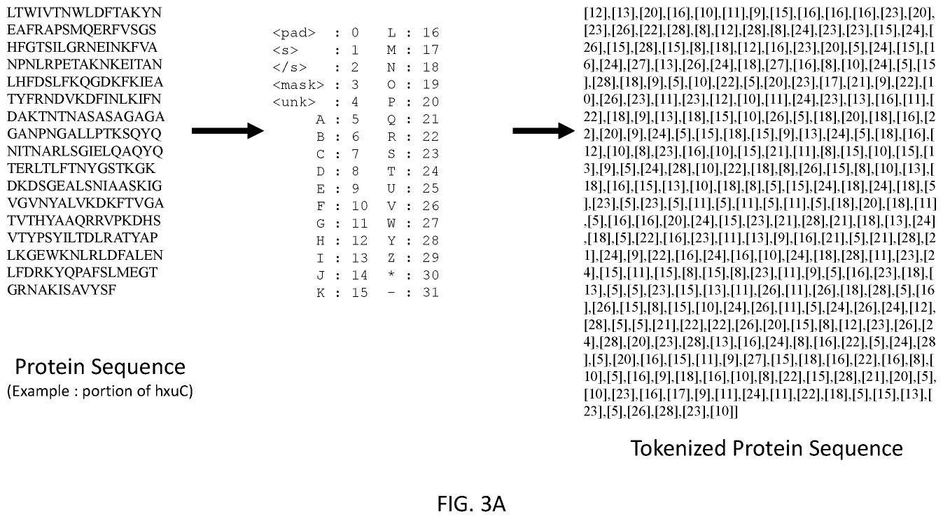

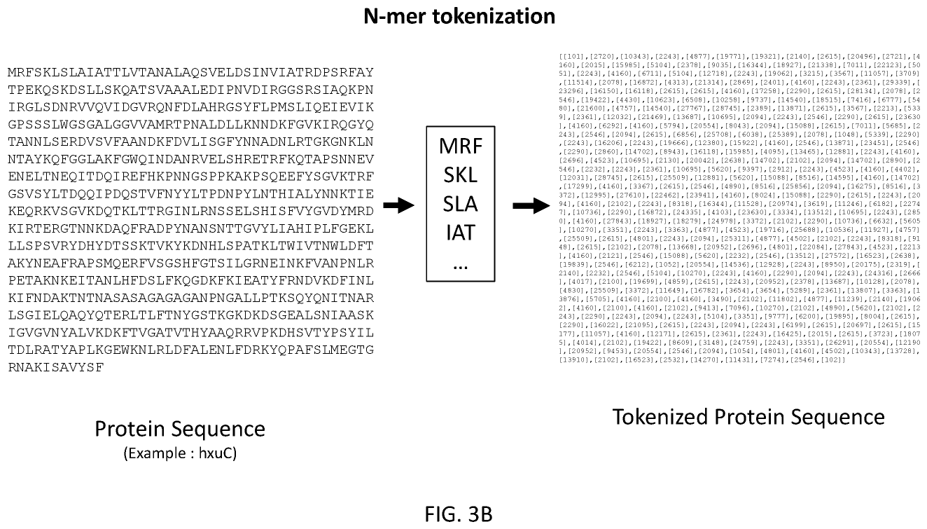

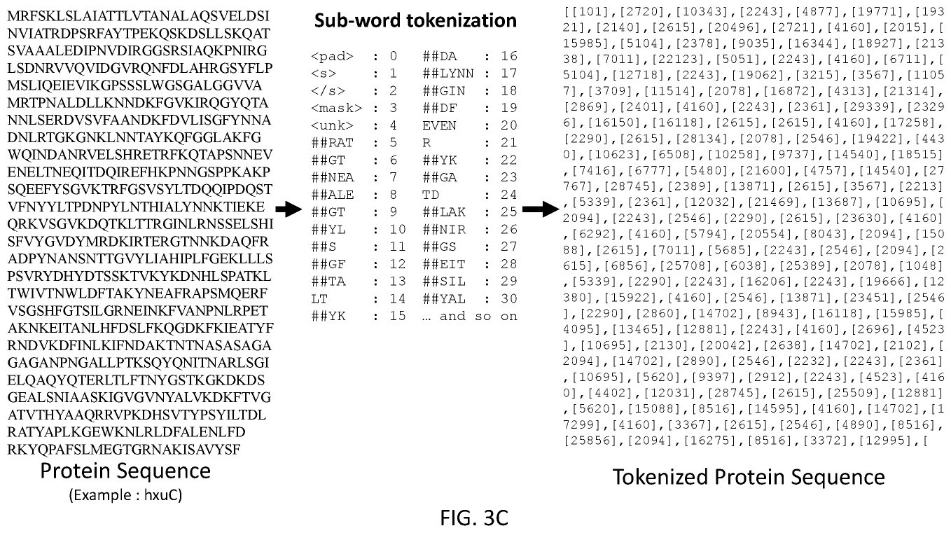

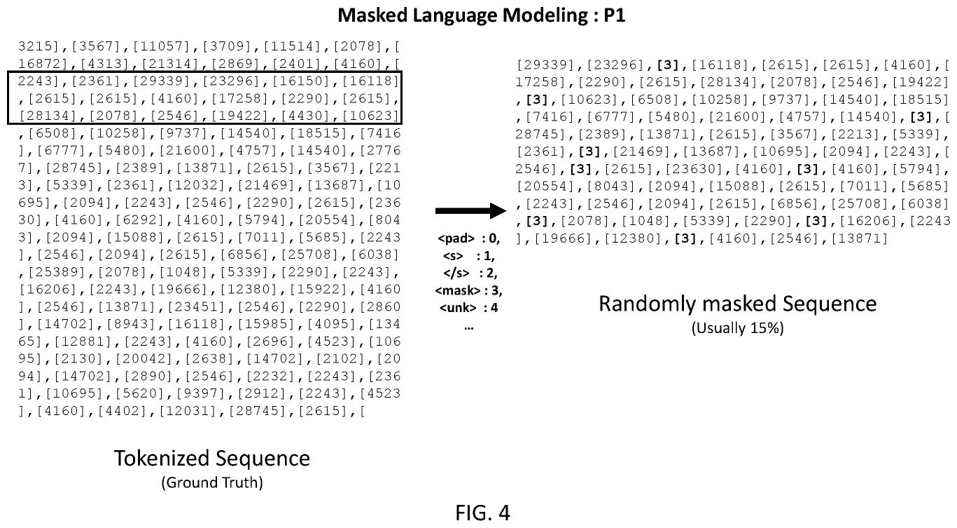

Tokenizer module 215 performs tokenization on the amino acid sequences. Tokenizer module 215 receives the output of the ingestion module (e.g., comprising a data structure or structured data with each entry of an array comprising an identifier and a corresponding amino acid sequence), and converts the received data structure or structured data into a tokenized data structure. Tokenization of an amino acid sequence comprises parsing the amino acid sequence into components (e.g., individual amino acids, n-mers, subwords, etc.) and mapping each component to a value. For example, an amino acid sequence may be separated into individual amino acids, and each individual amino acid may be mapped to a numeric value (e.g., MRF . . . ->[17], [22], [10] . . . ). The output of the tokenizer module may be another data structure or structured data (e.g., comprising an array or matrix structure) with each entry corresponding to a tokenized representation of an amino acid sequence. In aspects, inputs may be embedded into the system via individual amino acid tokenization. (Other inputs may be treated as categorical variables that are not subject to individual amino acid tokenization). In some aspects, the components are mapped to numeric values to streamline processing. Various approaches for tokenization and masking that may be utilized in accordance with training the protein language NLP system are provided below with reference to FIGS. 3A-3C and FIG. 4.

With reference to FIG. 3A, in some aspects, tokenization may be performed at the individual amino acid-level (individual amino acid-based tokenization), also referred to as single or individual amino acid-level tokenization, in which each individual amino acid is mapped to a different numeric value. For example, the amino acid for alanine “A” may be mapped to a numeric value “5,” the amino acid for valine “V” may be mapped to a numeric value “26”, and so forth. Other characters such as various types of whitespace characters (e.g., padding, space, tab, return, etc.), unknown characters, wildcard characters, hyphens, etc. may each be mapped to other numeric values. An example of an individual amino acid-level tokenization scheme is provided in FIG. 3A.

With reference to FIG. 3B, in another aspect, n-mer tokenization may be performed. In this approach, short n-mers of adjacent amino acids (e.g., where n is a numeric value such as 2, 3, 4 or more to form respective strings of two amino acids, three amino acids, four amino acids, etc. or any combination thereof), are each mapped to numeric values. Examples of n-mers include but are not limited to: two-mers such as AV, LK, CY, NW, etc., three-mers such as ASK, SKJ, VAL, TGW, JHS, etc. and so forth.

Referring to FIG. 3C, in another aspect, sub-word tokenization may be performed. In this approach, sub-words or strings of amino acids of varying length are each mapped to particular numeric values. Examples of sub-words include but are not limited to: ##RAT, ##GT, R, TD, ##LYNN, etc. In some aspects, sub-words are determined based upon analysis of protein sequences or may be based upon knowledge from the literature and/or subject matter experts.

Sequences may be tokenized according to any suitable tokenization scheme provided herein.

Once tokenized, one or more sequences may be subject to masking, which hides the identity of amino acids at random locations in the amino acid sequences. With reference to FIG. 2, data masking module 220 may mask amino acid sequences, obscuring the identity of amino acids at random locations to create a training dataset (e.g., masked training sequences 147) for the first neural network 225. Data masking module may receive as input, the output of the tokenizer module (e.g., a data structure or structured data comprising an array or matrix structure) with each entry comprising a tokenized representation of an amino acid sequence. The data masking module may utilize a masking function that randomly selects amino acids (e.g., a percentage of amino acids within a protein sequence) and masks the identity of these amino acids. Masking hides the identity of amino acids at particular locations in the sequence. For example, an amino acid at a given position may be known to be a valine, and the data masking module hides or obfuscates the identity of the amino acid at this position by replacing it with a designated masking value. The output of the data masking module may comprise another data structure or structured data, for example, comprising an array or matrix of entries (e.g., sequences), with each entry including a masked and tokenized amino acid sequence, which is provided as input to a first neural network for a first phase of training.

For example, and with reference to FIG. 4, a tokenized sequence in which individual amino acids are represented as numeric values is masked, with the masked amino acids represented by a designated masking value (e.g., in this case, <3>). In some aspects, about 5%, 6%, 7%, 8%, 9%, 10%, 11%, 12%, 13%, 14%, 15%, 16%, 17%, 18%, 19%, 20%, 21%, 22%, 23%, 24%, 25% of amino acid sequences, collectively across the library of proteins may be masked or any range therein. In other aspects, for TCR sequences, about 1, 2, 3, 4 or 5 amino acids per sequence may be masked. For example, masking may be applied to an individual epitope or TCR sequence, masking from about 1 to 5 amino acid residues (e.g., with shorter sequences having fewer absolute numbers of masked amino acids than longer sequences) or masking may be applied across the library (without regard to individual proteins) such that 15% of the total number of amino acids in the library are masked.

In some aspects, between 5-25% of the protein sequence, between 10-20%, between 11-19%, between 12-18%, between 13-17%, between 14-16%, or about 15% of the dataset is masked.

In some aspects, amino acids are masked in a random manner In still other aspects, masking may be constrained such that the masked amino acids are not permitted to be adjacent to each other. For example, the masked amino acids may be separated by a minimum spacing such that there is 1, 2, 3, 4, 5, 6, etc. unmasked amino acids between masked amino acids.

Referring back to FIG. 2A, a first neural network 225 is provided for training with the masked, tokenized, TCR and epitope datasets (e.g., masked training sequences 147). The first neural network 225 may comprise any suitable NLP model 235 including but not limited to deep learning models designed to handle sequential data. For example, the first neural network may comprise one or more of a transformer model, a generative model, a LSTM model, a shallow neural network model, etc. In aspects, the first neural network may comprise a first transformer and a second transformer (e.g., as provided in machine learning algorithm 236). The first transformer may be trained with an epitope sequence dataset, with the corresponding model parameters stored in machine learning parameters 237. In another aspect, a second transformer may be trained with a TCR sequence dataset, with the corresponding model parameters stored in machine learning parameters 237.

The first neural network 225 may be trained on one or more dataset (e.g., publicly available, privately available, or a combination thereof) and with or without preprocessing in a self-supervised manner Self-supervised training may proceed until meeting suitable criteria, for example, as specified by an AUC-ROC curve. For example, training may continue until reaching an AUC value of 0.7, 0.75, 0.8, 0.85, 0.90, 0.95, 0.96, 0.97, etc. An annotated dataset is not needed for training the first neural network, since the next amino acid is known (except for the end of the amino acid sequence).

In some aspects, transformer models, which are suitable for understanding context and long-range dependencies during self-supervised learning on large datasets are preferred. Transformer models with attention have been applied to the English language (see, Vaswani et al., “Attention is All you Need,” (2019) arXiv:1706.03762v5).

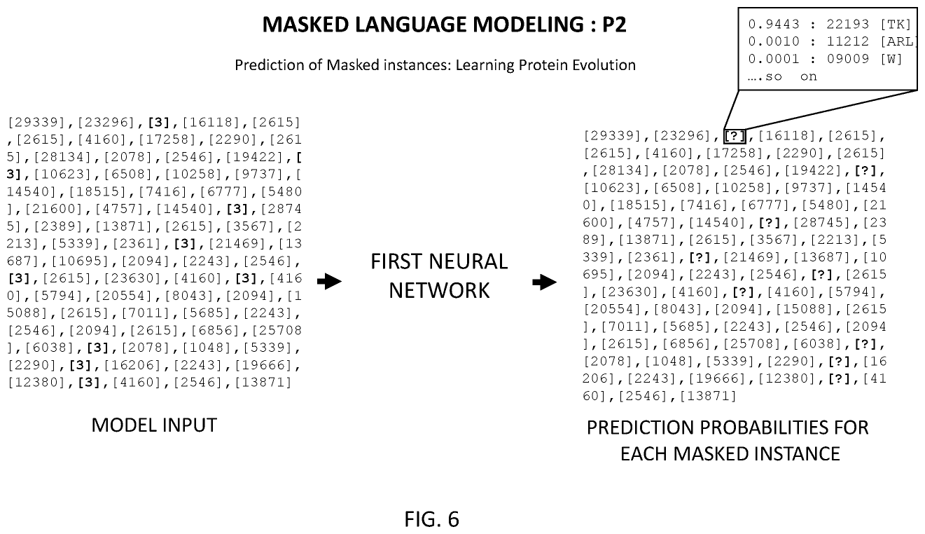

In some aspects, an attention-based transformer (e.g., self-attention) may be trained on masked, tokenized protein sequences, wherein the protein sequences have been masked to obscure about 15% of the amino acid identities within a given dataset. During training, the first neural network makes a determination of the masked value based upon a statistical likelihood. As the identity of the amino acid (true value) is known, the predicted amino acid identity may be compared to the true value, and this information may be provided back to the first neural network to improve the predictive ability of the first neural network. The output of the attention-based transformer model corresponds to another data structure or structured data, e.g., comprising entries of masked, tokenized amino acid sequences, with each masked amino acid instance associated with a probability of a specific amino acid at the masked position (see, FIG. 6). In aspects, a first transformer model is trained on an epitope sequence datasets separately from a second transformer model that is trained on TCR sequence datasets.

In some aspects, the protein language model may be trained on sequences limited to human origin or mammalian origin. In other aspects, the protein language model may be trained on sequences limited to human origin.

Once the first neural network 225 has been trained, the knowledge (e.g., neural network configuration and hyperparameters stored in machine learning parameters 237) obtained from this process may be provided to cross attention module 240, which computes cross attention between TCR sequences and epitope sequences (see, FIGS. 2B-2D) to improve binding predictions between TCR sequences and epitopes.

For example, the output of the first transformer may comprise a vector output including information from training the first transformer on TCR sequences and the second transformer may comprise another series of vectors with information from training the second transformer on epitope sequences. For computing cross attention, a subset of the vector output from the first transformer may be combined with a subset of output from the second transformer to compute cross attention between TCR embeddings and epitope embeddings. A different subset of vectors from the output of the first transformer may be combined with a different subset of vectors from the second transformer to compute cross attention between epitope embeddings and TCR embeddings. Thus, cross attention may be computed between TCR embeddings and epitope embeddings and between epitope embeddings and TCR embeddings by combining different subsets of vector outputs from each of the first and second transformer models. This is described in additional detail below. The output of the cross attention module 240 may be provided to a second neural network 230 (e.g., using transfer learning module 245), and the second neural network may be fine-tuned through further training.

In aspects, the second neural network 230 may comprise any suitable NLP model 235 including but not limited to deep learning models designed to handle sequential data. For example, the second neural network may comprise a fully connected neural network, a perceptron, a transformer model, a generative model, a LSTM model, a shallow neural network model, etc. In aspects, the output layer of the second neural network may comprise a classifier or a (multi-output) regression model that predicts binding affinity between an epitope sequence and a TCR sequence.

With respect to the present application, the first neural network refers to the neural network which is trained in a first phase on TCR sequences and epitope sequences, and may include computing cross attention between TCR sequences and epitope sequences. The second neural network refers to a neural network containing information from the first phase of training and trained in a second phase on an annotated, compact, specific dataset. Any suitable technique in transfer learning may be used to transfer knowledge from the first neural network to the second neural network.

Thus, a second neural network may refer to a neural network comprising information from the first neural network using any suitable approach (e.g., loading, copying, transferring embeddings/layers, custom generated data functions/variables, embeddings, concatenated representations of sequence and categorical feature embeddings from the output of the first neural network, categorical variables, etc.) that is further fine-tuned in a second phase for to predict binding affinity between TCRs and epitopes. In further aspects, hyperparameters (e.g., weights, layers, labels, inputs, outputs, parameters, variables, etc.) obtained from the trained first neural network may be transferred or loaded into a second neural network.

In other aspects, processing may continue using a modified form of the first neural network. These changes may include replacing one or more output layers of the trained first neural network with replacement layers, such that the resulting neural network comprises layers (retained layers) from the first phase of training and replacement layers that replace one or more output layers. In some aspects, model constraints 246 are applied to the retained layers to prevent or constrain modification of the weights of these layers during subsequent training with an annotated dataset, which tailors or fine-tunes the second neural network for a specific task (see, e.g., https://keras.io/guides/transfer_learning/). Model constraints 246 ensure that information from the first phase of training is retained by reducing/minimizing parameter changes for the retained layers. In some aspects, one or more retained layers may be released in a layer-by-layer manner as training progresses to allow small scale parameter modification.

In other aspects, data may be transferred by obtaining embeddings from the first phase of training and providing the embeddings (in any suitable form including embeddings/layers, custom generated data functions/variables, embeddings, concatenated representations of sequence and categorical feature embeddings, categorical variables, etc.) to the second neural network for the second phase of training.

Thus, the second training phase fine-tunes the second neural network to a specific application (e.g., binding affinity). Training may proceed until a specified AUC-ROC parameter has been met. In some aspects, the output layer of the second neural network determines whether a physiological feature is present (e.g., the output layer may act as a classifier regarding presence of binding or no binding, level of binding, etc.). In some aspects, the output layer comprises a measure of the specified feature, for example, a measure of binding affinity (e.g., high, medium, low, etc.) between a receptor and a ligand (e.g., between a TCR receptor and an epitope).

Display module 250 provides various displays for users to visualize and explore the output of the protein language NLP system. For example, display module 250 comprises a salience module 254 (interpretability module), which provides interpretability into the protein language NLP system, providing users with insights into predictions at the amino acid level. Using this salience module, a user may gain insight into which amino acids of the protein sequence contributes to a specific biophysiochemical property (e.g., TCR-epitope interactions). In some aspects, the contributory amino acids are individually highlighted according to a color schema (e.g., red to blue, light to dark, etc.) in the display.

In other aspects, feature ranking module 254 may rank the output of the second neural network for a plurality of candidate amino acid sequences, based on any suitable parameter, e.g., strength of binding affinity, etc.

FIG. 2B shows an overview of a protein language modeling pipeline for training RoBERTa/BERT-style transformer language models with a cross-attention module on large corpora of TCR amino acid sequences and epitope sequences to predict binding probabilities between epitope and TCR sequences.

TCR dataset 2210 and/or epitope dataset 2220 may undergo data preprocessing (see, e.g., FIGS. 19A and 19B), tokenization at the character level, and masking at the character level (see also, e.g., FIGS. 3A-6) before being provided as input to an attention-based (e.g., self-attention) transformer (e.g., in some aspects, RoBERTa LM 2230 or 2240).

In a first phase of training, the TCR sequence dataset 2210 is provided to RoBERTa language model (LM) 2230, which is trained on TCR (CDR3) sequences; the epitope sequence dataset 2220 is provided to RoBERTa language model (LM) 2240, which is trained on epitope sequences. In this example, the RoBERTa styled language model (LM) may be trained in a self-supervised manner with CDR3β or epitope sequences to learn the probability distribution of amino acids in a given TCR or epitope sequence. The tokenized sequences may be provided as input to a transformer model, and converted to a sum of token and positional vector embeddings, which are then processed by alternating layers of self-attention and feed-forward modules in the transformer model (e.g., RoBERTa LM architecture). RoBERTa models are generally described in Liu et al. (RoBERTa: A Robustly Optimized BERT Pretraining Approach; arxiv.org/abs/1907.117692).

This system has four attention blocks 2240, 2230, 2250, 2251 (e.g., with the same architecture and size, and different weights, see https://arxiv.org/abs/1706.03762). The first two blocks compute self-attention (e.g., (ep-ep) and (tcr-tcr)). The second two blocks compute cross-attention (e.g., (ep-tcr) and (tcr-ep)). The two feedforward layers 2252, 2253 are of the same size, and transform the inputs independently from all other inputs provided to the cross-attention module. The sums and the lines connecting inputs to outputs represent skip connections and their role is to improve training.

For each tokenized sequence dataset, a random subset of tokens may be selected to serve as training target labels/training data. For example, the transformer may be trained to predict amino acid residues at specified positions, and predictions may be verified based upon known sequences. A second subset may be replaced with a random token (e.g., to corrupt the value of the amino acid residue), and used during training to improve predictive capabilities as well.

For this self-attention model (e.g., RoBERTa), the input to the transformer may also be added to the output of the transformer via a skip connection module to improve e.g., convergence during training. Skip connections may skip one or more layers in a neural network to provide the output of one layer as input to another layer—in this example, the input is provided to an output layer. The transformer model generates a probability distribution over the amino acid token vocabulary for each token in the training dataset by acting as a prediction head on the final contextualized token embeddings. In aspects, training involves optimizing the cross-entropy loss between the model's predictions and the target labels (see also, FIG. 3A to FIG. 6).

Information from the pretrained RoBERTa LMs may be transferred downstream for further refinement of TCR and epitope binding predictions. The outputs of the RoBERTa language models (LM) 2230 and 2240 are passed to cross-attention module 2250, 2251, which computes cross attention between embedded TCR sequences and embedded epitope sequences to improve predictive capabilities. This downstream processing utilizes cross-attention mechanisms that include as inputs, tuples of epitope and TCR-CDR3β sequences, which are individually embedded using the (pretrained) RoBERTa based language model. An embodiment of cross attention module 2250, 2251 is described in additional detail below (see, FIG. 2D).

The output of the cross attention module 2250, 2251 is provided to fully connected neural network 2260 (e.g., a multilayer perceptron). Concatenated representations of sequence and categorical feature embeddings from the cross attention module are appended and provided as input to fully connected neural network 2260. During the second phase of training, the fully connected neural network 2260 is fine-tuned to generate TCR-epitope binding probabilities 2270 (e.g., binding probabilities between tuples of TCR sequences and epitope sequences). A classification head outputs binding probabilities by acting with a multilayer perceptron on the concatenated representations of sequence and categorical feature embeddings (e.g., from the cross-attention module). Thus, the protein language NLP model is trained end-to-end to generate predictions 2270 (binding probabilities) for a given TCR and epitope sequence (e.g., tuple).

FIG. 2C shows an illustration of a protein language NLP processing pipeline according to the embodiments provided herein. In this figure, TCR sequence datasets and epitope sequence datasets are provided to a RoBERTa language model (e.g., two RoBERTa language models may be present, one is shown here for simplicity). The first RoBERTa language model trained on a TCR sequence dataset and the second RoBERTa language model trained on an epitope sequence dataset. After training, the output of the TCR RoBERTa model and the epitope RoBERTa model are provided to a cross attention module. Cross attention is computed between the epitope input and the TCR input, as well as between the TCR input and the epitope input. The output of the cross attention module is provided to a fully connected neural network. Input to the fully connected neural network may be in the form of embeddings, concatenated representations of sequence and categorical feature embeddings, categorical variables or any combination thereof. The fully connected network outputs predictions of binding affinities between tuples of epitopes and TCRs.

FIG. 2D shows cross attention module 2300. A description of cross attention architectures are generally described in Wei et al. (Multi-Modality Cross Attention Network for Image and Sentence Matching (2020), Computer Vision Foundation, 10941-10950). The output of the transformer-based (RoBERTa) LM module 2230 (see, FIG. 2B) may be provided to cross attention module 2300 as TCR embeddings 2310. Similarly, the output of the transformer-based (RoBERTa) LM module 2240 may be provided to cross attention module 2300 in the form of epitope embeddings 2315. In aspects, the output of transformer models, such as from RoBERTa models, may comprise vectors (e.g., query vectors, key vectors, and value vectors). To compute cross attention, vectors from the TCR embeddings 2310 and from the epitope vectors 2315 may be provided as input to an attention module 2320, and a different subset of vectors from the TCR embeddings 2310 and from the epitope embeddings 2315 may be provided as input to another attention module 2330. For example, a query vector from TCR embeddings and key/value vectors from epitope embeddings may be provided to attention module 2320; key/value vectors from TCR embeddings and query vectors from epitope embeddings may be provided to attention module 2330. The output of the attention modules 2320, 2330 may be provided as input to feed forward module(s) 2340, which provide input to fully connected neural network 2260.

One or more feed forward layers of feed forward module(s) 2340 may be used to pass epitope and TCR embeddings as input to neural network 2260. The feed forward layers (see, FIG. 2D), provide input to the neural network 2260 from the attention modules 2320, 2330. Thus, the output of the cross attention module 2300 is provided to neural network 2260, which processes the input (e.g., tuples of an epitope and TCR) to generate a binding prediction 2270. These embodiments are intended to be exemplary, and in no way should be construed as limited to the embodiments provided herein.

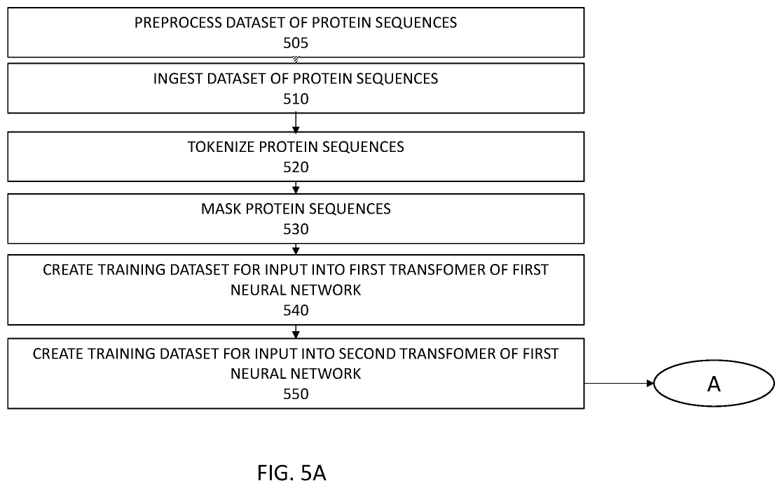

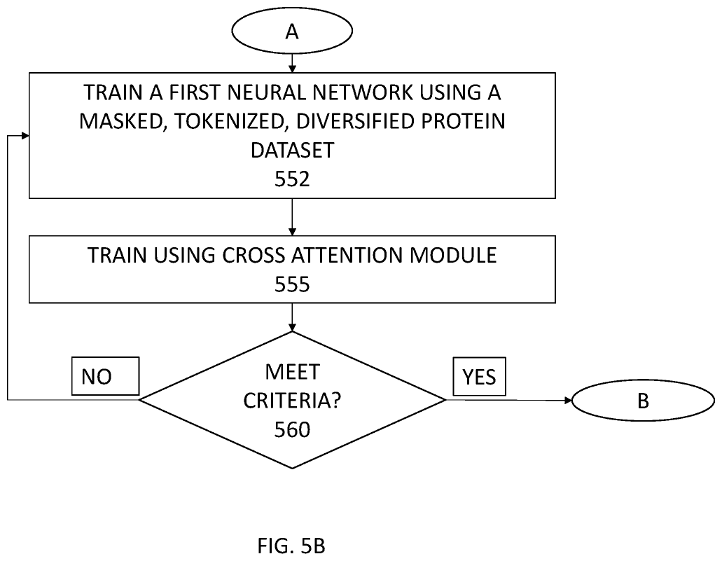

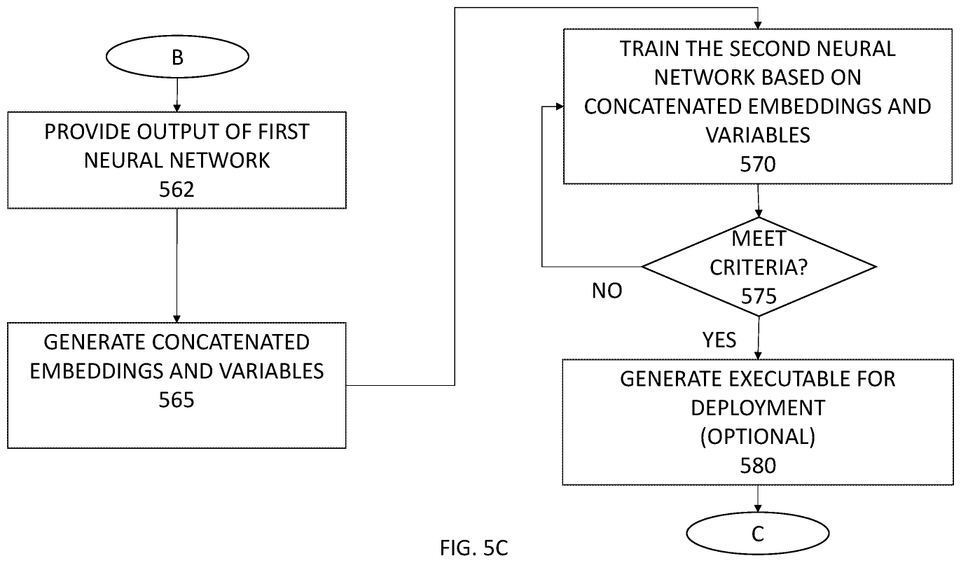

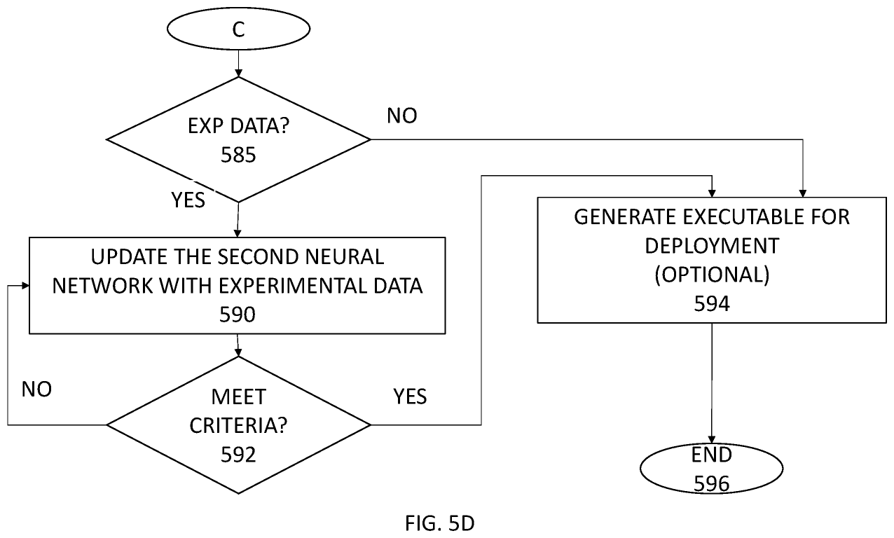

FIGS. 5A-5D provide flow diagrams for generating a trained protein language NLP system, according to embodiments of the present disclosure. FIG. 5A is a flow diagram for operations involving generating a training data set for training a protein language NLP system according to embodiments of the present disclosure. At operation 505, protein sequences are preprocessed (e.g., TCR sequences and epitope sequences). The sequences may undergo preprocessing according to FIGS. 19A and 19B prior to ingestion. At operation 510, protein sequences are ingested. Protein sequences may include epitope sequence datasets, TCR sequence datasets from public or private databases, such as internal databases, or any combination thereof. The protein sequences may be provided in any suitable format including FASTA, SAM, etc. At operation 520, the protein sequences undergo tokenization, for example, individual amino acid-level tokenization, n-mer tokenization or sub-word tokenization. At operation 530, the tokenized sequences are subjected to a masking process, for example, in which about 15% of the amino acids of the dataset are masked (e.g., 5-25%, 12-17%, 15%). In aspects, data processing at this stage may also optionally include a low level of data corruption, in which amino acids are replaced by other amino acids in a random manner. At operation 540, the training dataset for TCR sequences is generated for input into the first transformer of the first neural network. At operation 550, the training dataset for epitope sequences is generated for input into the second transformer of the first neural network. Label “A” from FIG. 5A continues to Label “A” on FIG. 5B.

Continuing to FIG. 5B, at operation 552, a first neural network (e.g., one or more transformer model(s) with attention) is trained, e.g., in a self-supervised manner, using a masked, tokenized, protein dataset. For example, this includes training the first transformer with a TCR sequence dataset and the second transformer with an epitope sequence dataset. At operation 555, training continues by computing cross attention with a cross attention module based on the output of the first and second transformer models—between the embeddings of the TCR sequences and the epitope sequences. At operation 560, the system determines whether suitable training criteria, such as AUC-ROC criteria, have been met. If not, training may continue at operation 552. Otherwise, training is terminated. Label “B” from FIG. 5B continues to Label “B” on FIG. 5C.

With respect to FIG. 5C, at operation 562, the output of the first neural network is provided. At operation 565, concatenated embeddings and variables are generated. At operation 570, the second neural network is trained by providing the concatenated embeddings and variables as input to the second neural network. The second neural network is trained using a specific, compact annotated dataset in a supervised manner. At operation 575, the system determines whether suitable training criteria have been met (e.g., AUC-ROC, etc.). If not, training may continue at operation 570. If criteria have been met, training is terminated. In some aspects, at operation 580, an executable for deployment on a client system may be generated (optional). Label “C” of FIG. 5C continues to Label “C” on FIG. 5D.

With reference to FIG. 5D, at operation 585, the system determines whether experimental data is available. If no data is available, training operations may cease. At operation 594, an executable for deployment may be generated (optional), and at operation 596, the process ends.

If data is available, additional training of the second neural network may occur with the experimental data at operation 590. At operation 592, the system determines whether suitable training criteria have been met. If not, training may continue at operation 590. Otherwise, training is terminated. At operation 594, an executable for deployment may be generated (optional). The process ends at operation 596. Operations 585-596 may be repeated as additional data becomes available.

Additionally, the first neural network may be updated as new protein sequences become available, and the second neural network retrained accordingly.

FIG. 6 shows output probabilities at masked amino acid positions, according to embodiments of the present disclosure, based on the first phase of training the protein language NLP system.

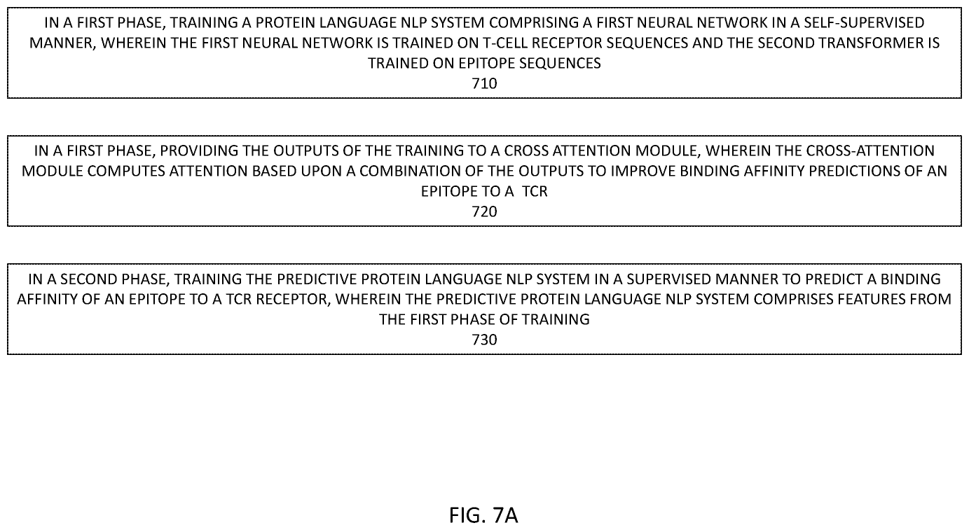

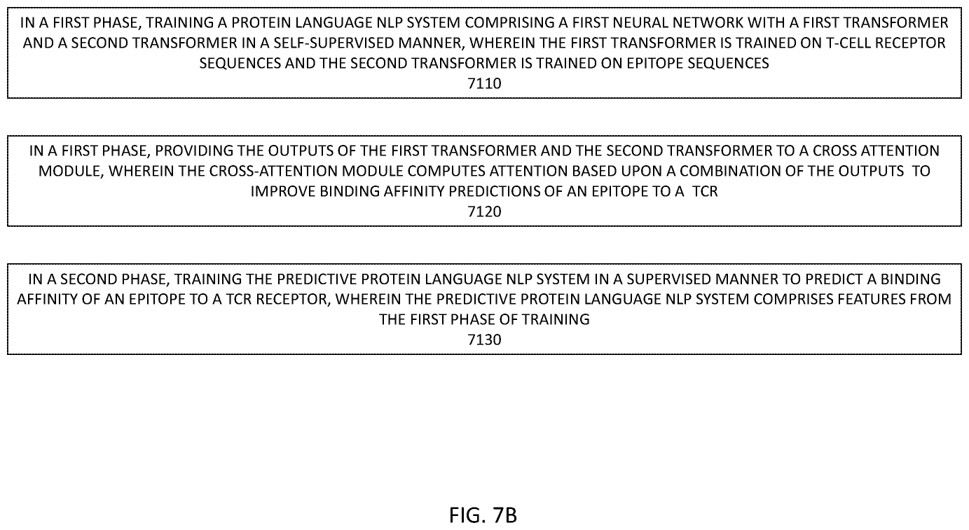

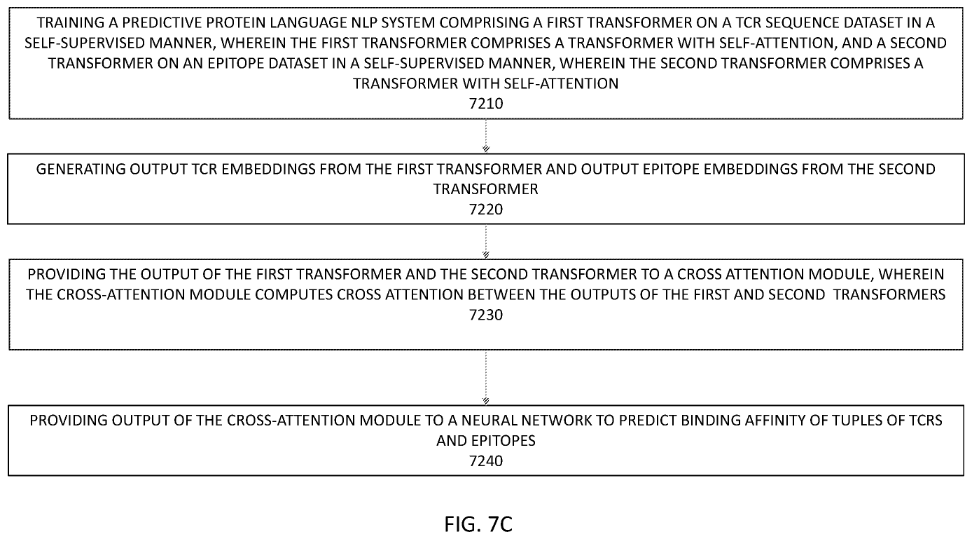

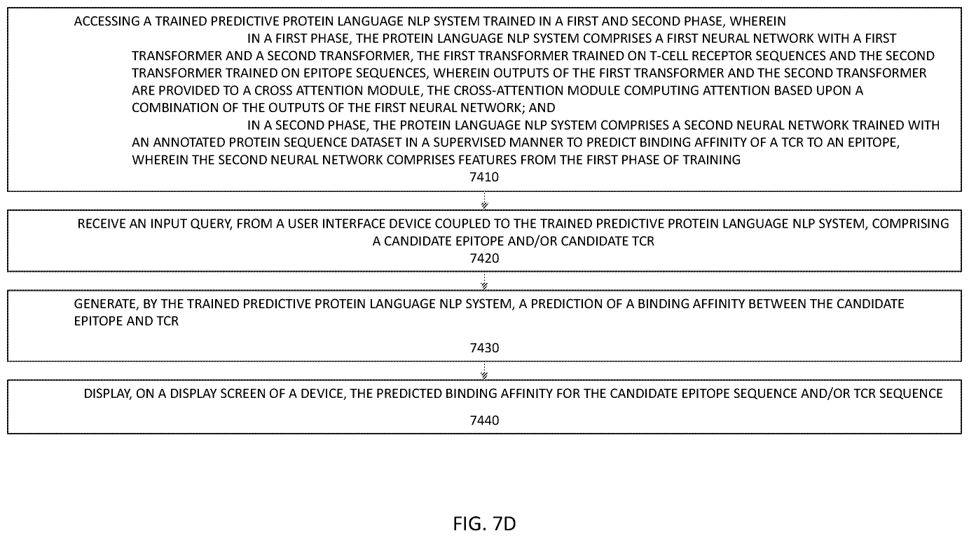

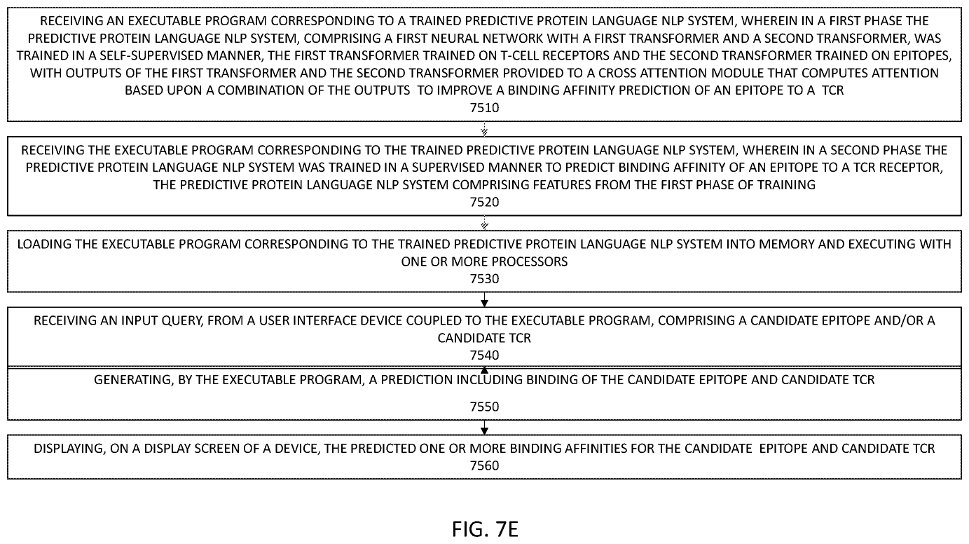

FIGS. 7A-7E show example flowcharts of operations for training and utilizing the protein language NLP system according to the embodiments provided herein.

FIG. 7A is a high-level flowchart of example training operations of the protein language NLP system, in accordance with the embodiments provided herein. At operation 710, in a first phase, a protein language NLP system comprising a first neural network is trained in a self-supervised manner, wherein the first neural network is trained on T-cell receptor sequences and the second transformer is trained on epitope sequences. At operation 720, in a first phase, the outputs of the training of the first neural network are provided to a cross-attention module, wherein the cross-attention module computes attention based upon a combination of the outputs (e.g., TCR embeddings and epitope embeddings) to improve binding affinity predictions of an epitope to a TCR. At operation 730, in a second phase, the predictive protein language NLP system, wherein the predictive protein language NLP system comprises features from the first phase of training, is trained with an annotated protein sequence dataset in a supervised manner to predict a binding affinity of an epitope to a TCR, wherein the annotated protein sequence dataset comprises known tuples of TCRs and epitopes with known binding affinities.

FIG. 7B is another flowchart of example operations for training a protein language NLP system that predicts a binding affinity of an antigen to a TCR epitope, in accordance with the embodiments provided herein. At operation 7110, in a first phase, a protein language NLP system comprising a first neural network with a first transformer and a second transformer is trained in a self-supervised manner, wherein the first transformer is trained on t-cell receptor sequences and the second transformer is trained on epitope sequences. At operation 7120, in a first phase, the outputs of the first transformer and the second transformer are provided to a cross-attention module, wherein the cross-attention module computes attention based upon a combination of the outputs to improve binding affinity predictions of an epitope to a TCR. At operation 7130, in a second phase, the predictive protein language NLP system is trained in a supervised manner to predict a binding affinity of an epitope to a TCR receptor, wherein the predictive protein language NLP system comprises features from the first phase of training.

FIG. 7C is a flowchart of example operations for an executable corresponding to a trained predictive protein language NLP system, in accordance with the embodiments provided herein. At operation 7210, a predictive protein language NLP system comprising a first transformer is trained on a TCR sequence dataset in a self-supervised manner, wherein the first transformer comprises a transformer with self-attention, and a second transformer is trained on an epitope dataset in a self-supervised manner, wherein the second transformer comprises a transformer with self-attention. At operation 7220, output TCR embeddings are generated from the first transformer and output epitope embeddings are generated from the second transformer. At operation 7230, the outputs of the first transformer and the second transformer are provided to a cross-attention module, wherein the cross-attention module computes cross-attention between outputs of the first and second transformer. At operation 7240, the output of the cross-attention module is provided to a neural network to predict binding affinity of tuples of TCRs and epitopes.

Thus, in aspects, a trained predictive protein language NLP system comprising a first neural network with a first transformer and a second transformer, trained in a first phase in a self-supervised manner, may be accessed by a user, wherein the first transformer is trained on T-cell receptor sequences and the second transformer is trained on epitope sequences, and wherein outputs of the first transformer and the second transformer are provided to a cross-attention module. As provided herein, the cross-attention module computes attention based upon a combination of the outputs to improve binding affinity predictions of an epitope and a TCR.

FIG. 7D is a flowchart of example operations for a trained protein language NLP system, in accordance with the embodiments provided herein. At operation 7410, a trained protein language NLP system is accessed, wherein the system was trained in a first phase (the protein language NLP system comprising a first neural network with a first transformer and a second transformer), the first transformer trained on t-cell receptor sequences and the second transformer trained on epitope sequences, and wherein outputs of the first transformer and the second transformer were provided to a cross attention module, the cross-attention module computing attention based upon a combination of the outputs of the first neural network; and in a second phase, the protein language NLP system comprising a second neural network trained with an annotated protein sequence dataset in a supervised manner to predict binding affinity of a TCR to an epitope, wherein the second neural network comprises features from the first phase of training.

At operation 7420, an input query is received, from a user interface device coupled to the trained protein language NLP system, comprising a candidate epitope and/or TCR. At operation 7430, the trained predictive protein language NLP system generates a prediction of a binding affinity between the candidate epitope and TCR. At operation 7440, the predicted binding affinity for the candidate epitope sequence and/or TCR sequence is displayed on a display screen of a device.

FIG. 7E is another flowchart of example operations for accessing a trained protein language NLP system that predicts a binding affinity of an antigen to a TCR epitope, in accordance with the embodiments provided herein. At operation 7510, an executable program corresponding to a trained predictive protein language NLP system is received, wherein the predictive protein language NLP system comprises a first neural network with a first transformer and a second transformer trained in a first phase in a self-supervised manner, the first transformer trained on T-cell receptor sequences and the second transformer trained on epitope sequences, with outputs of the first transformer and the second transformer provided to a cross attention module that computes attention based upon a combination of the outputs to improve a binding affinity prediction of an epitope to a TCR. At operation 7520, the executable program corresponding to a trained predictive protein language NLP to predict binding affinity of an epitope to a TCR receptor is received, wherein in a second phase the predictive protein language NLP system was trained in a supervised manner to predict binding affinity of an epitope to a TCR receptor, the predictive protein language NLP system comprising features from the first phase of training. At operation 7530, the executable program corresponding to the trained predictive protein language NLP system was loaded into memory and executed with one or more processors. At operation 7540, an input query was received from a user interface device coupled to the executable program, comprising a candidate epitope and/or a candidate TCR. At operation 7550, the executable program generated a prediction including binding of the candidate epitope and candidate TCR. At operation 7560, the predicted one or more binding affinities for the candidate epitope and candidate TCR are displayed, on a display screen of a device.

In aspects, a trained protein language NLP system is provided, wherein the system comprises a neural network that predicts binding affinity of tuples of TCRs and epitopes. TCR embeddings are generated from a first transformer and the epitope embeddings are generated from a second transformer. A cross-attention module determines cross-attention between TCR embeddings and epitope embeddings. This information is transferred to a second neural network, which outputs predictions of binding affinity and/or a levels of binding affinity.

In aspects, the protein language NLP system allows a library of TCR sequences and epitopes to be provided as input into a trained protein language NLP system, and for the system to predict which, if any, of the epitope bind with high affinity to the TCR. The results may identify epitopes (e.g., for vaccine design) to be selected based upon desired binding affinity.

In aspects, the library of therapeutic candidates (e.g., epitopes) may be hypothetical (not yet synthesized) and the output of the protein language NLP system may select candidates predicted to have certain properties. These candidates may be synthesized and experimentally validated.

In aspects, the protein language NLP system may be applied to the field of biotechnology and adapted to the specific technical implementation of predicting TCR epitope binding and/or a degree of binding, e.g., with a confidence interval according to an AUC criteria as determined by statistical approaches.

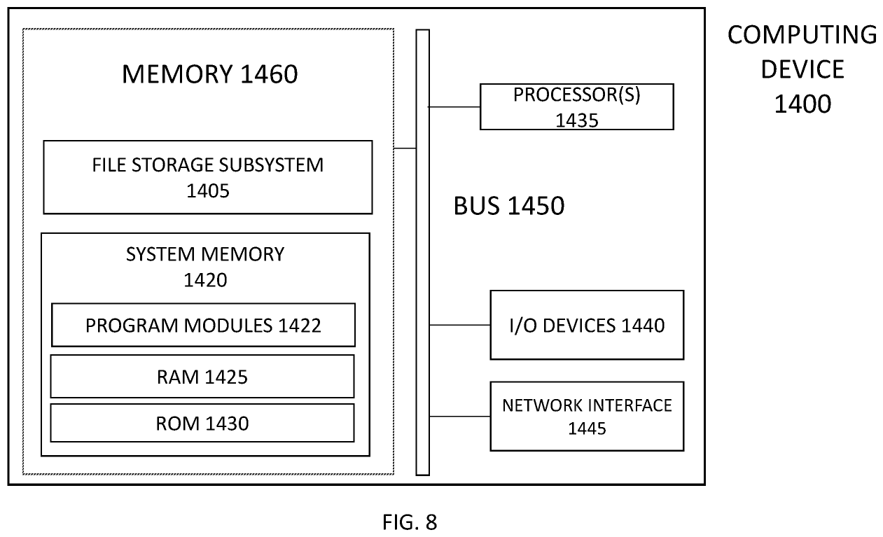

FIG. 8 is an example architecture of a computing device 1400 that may be used to perform one or more aspects of the protein language NLP system described herein. Components of computing device 1400 may include, without limitation, one or more processors (e.g., CPU, GPU and/or TPU) 1435, network interface 1445, I/O devices 1440, memory 1460, and bus subsystem 1450. Each component is described in additional detail below. It is to be understood that the software (e.g., protein language NLP system of the present embodiments) may be implemented in any desired computer language and may be developed by one of ordinary skill in the computer arts based on the functional descriptions contained in the specification and flow charts illustrated in the drawings. Further, any references herein of software performing various functions generally refer to computer systems or processors performing those functions under software control. The computer systems of the present embodiments may alternatively be implemented by any type of hardware and/or other processing circuitry.

In aspects, the client device may be located in a first geographical location, and the server device housing the protein language NLP system may be located in a second geographical location. In another aspect, the client device and the server device housing the protein language NLP system may be located within a defined geographical boundary. In another aspect, an executable corresponding to the trained protein language NLP system may be generated in a first geographical location, and downloaded and executed by a client device in a second geographical location.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, and/or any other intermediary processing devices. The software and/or algorithms described above and illustrated in the flow charts may be modified in any manner that accomplishes the functions described herein. In addition, the functions in the flow charts or description may be performed in any order that accomplishes a desired operation.

It should be appreciated that all combinations of the foregoing concepts and additional concepts are contemplated as being part of the subject matter disclosed herein. All combinations of claimed subject matter appearing at the end of this disclosure are contemplated as being part of the subject matter disclosed herein. It will be appreciated that the embodiments described above and illustrated in the drawings represent only a few of the many ways of combining the features provided herein.

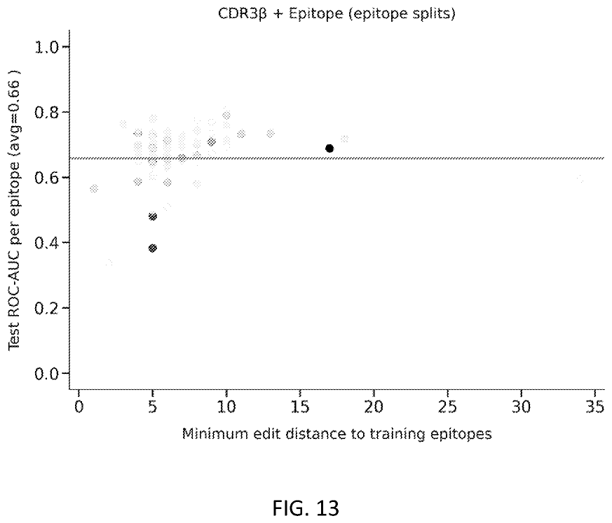

Computing device 1400 can any suitable computing device including a workstation, server, computing cluster, blade server, server farm, or any other data processing system or computing device. The example description of computer device 1400 depicted in FIG. 13 is intended only for purposes of illustrating some implementations. It is understood that many other configurations of computer system 1400, for example, with more or less components than as depicted in FIG. 13 are possible and fall within the scope of the embodiments provided herein.

It should also be understood that, although not shown, other hardware and/or software components may be used in conjunction with computing device 1400. Examples, include, but are not limited to: redundant processing units, external disk drive arrays, RAID systems, data archival storage systems, etc.

### Memory

Memory 1460 stores programming instructions/logic and data constructs that provide the functionality of some or all of the software modules/programs described herein. For example, memory 1460 may include the programming instructions/logic and data constructs associated with the protein language NLP system to perform aspects of the methods described herein. The programming instructions/logic may be executed by one or more processor(s) 1435 to implement one or more software modules as described herein. In embodiments, computing device 1400 may have multiple processors 1435, and/or multiple cores per processor.

Programming instructions/logic and data constructs may be stored on computer readable storage media. Unless indicated otherwise, a computer readable storage medium is a tangible device that retains and stores program instructions/logic for execution by a processor device (e.g., CPU, GPU, controller, etc.).

Memory 1460 may include system memory 1420 and file storage subsystem 1405, which may include any suitable computer readable storage media. In aspects, system memory 1420 may include RAM 1425 for storage of program instructions/logic and data during program execution and ROM 1430 used for storage of fixed program instructions. The software modules/program modules 1422 of the protein language NLP system contain program instructions/logic that implement the functionality of embodiments provided herein as well as any other program or operating system instructions and may be stored in system memory 1420 or in other devices or components accessible by the processor(s) 1435. File storage system 1405 may include, but is not limited to, a hard disk drive, a disk drive with associated removable media, or removable media cartridges, and may provide persistent storage for program instruction/logic files and/or data files.

A non-exhaustive list of examples of computer readable storage media may include any volatile, non-volatile, removable, or non-removable memory, such as: a random access memory (RAM), a dynamic random access memory (DRAM), a static random access memory (SRAM), a cache memory, a read-only memory (ROM), an erasable programmable read-only memory (EPROM or Flash memory), a portable computer diskette, a portable compact disc read-only memory (CD-ROM), a digital versatile disk (DVD), a floppy disk, a memory stick, or magnetic storage, a hard disk, hard disk drives (HDDs), or solid state drives (SSDs).

A computer readable storage medium is not to be construed as transitory signals, such as electrical signals transmitted through a wire, freely propagating electromagnetic waves, light waves propagating along a fiber, or freely propagating radio waves. The computer readable storage medium provided herein is non-transitory.

### I/O Devices

Input/output (I/O) device(s) 1440 may include one or more user interface devices that enable a user to interact with computing device 1400 via input/output (I/O) ports. User interface devices, which include input and output devices, may refer to any visual and/or audio interface or prompt with which a user may use to interact with computing device 1400. In some aspects, user interfaces may be integrated into executable software applications, programmed based on various programming and/or scripting languages, such as C, C#, C++, Perl, Python, Pascal, Visual Basic, etc. Other user interfaces may be in the form of markup language, including HTML, XML, or VXML. The embodiments described herein may employ any number of any type of user interface devices for obtaining or providing information.

Interface input devices may include any number of any types of input or actuation mechanisms (e.g., buttons, icons, fields, boxes, links, etc.) to enter/display information and initiate desired actions via any suitable input devices (e.g., mouse, keyboard, etc.). The interface screens may include any suitable actuators (e.g., links, tabs, etc.) to navigate between the screens in any fashion. User interface input devices may include a keyboard, pointing devices such as a mouse, a trackball, a touchpad, a graphics tablet, a scanner, a touchscreen incorporated into the display, audio input devices such as voice recognition systems, microphones, and/or any other suitable type of input device. In general, use of the term “input device” is intended to include all possible types of devices and ways to input information into computer device 1400 or any other suitable input device for receiving inputs from a user.

User interface output devices may include a display, including a cathode ray tube (CRT) display, a light emitting diode (LED) display, a liquid crystal display (LCD), an organic LED (OLED) display, a plasma display, a projection device, or other suitable device for generating a visual image. User interface output devices may include a printer, a fax machine, a display, or non-visual displays such as audio output devices. In general, use of the term “output device” is intended to include all possible types of devices and ways to output information from computer device 1400 to the user or to another machine or computer system.

### Network Interface

Computing device 1400 can communicate with one or more networks such as a local area network (LAN), a general wide area network (WAN), and/or a public or private network (e.g., the Internet, an Intranet, etc.) via network interface 1445. In some aspects, the network interface 1445 may be a wired communication interface that includes Ethernet, Gigabit Ethernet, or any suitable equivalent. In other embodiments, the network interface 1445 may be a wireless communication interface that includes modulators, demodulators, and antennas for a variety of wireless protocols including, but not limited to, Bluetooth, Wi-Fi, and/or cellular communication protocols for communication over a computer network. Network interface 1445 is accessible via bus 1450. As depicted, network interface 1445 communicates with the other components of computing device 1400, including processor 1435 and memory 1460, via bus 1450. The network interface allows the computing device 1400 to send and receive data through any suitable network.

### Bus

Bus subsystem 1450 couples the various computing device components together, allowing communication between various components and subsystems of memory 1460, processors 1435, network interface 1445, and I/O devices 1440.

Bus 1450 is shown schematically as a single bus, however, any combination of buses may be used with present embodiments. Bus 1450 represents one or more of any suitable type of bus structure, including a memory bus, a peripheral bus, an accelerated graphics port, or a local bus using any of a variety of bus architectures. By way of example, and without limitation, bus architectures may include Enhanced ISA (EISA) bus, Industry Standard Architecture (ISA) bus, Micro Channel Architecture (MCA) bus, Peripheral Component Interconnects (PCI) bus, and Video Electronics Standards Association (VESA) local bus.

### Program Instructions

The computer program product may include a computer readable storage medium (or media) having computer readable program instructions thereon for causing a processor to carry out aspects of the present invention.

Program modules 1422 may be stored in system memory 1420 by way of example, and not limitation, as well as an operating system, one or more application programs, other program modules, and program data. Program modules 1422 generally carry out the functions and/or methodologies of embodiments as described herein.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other devices implement the functions/acts specified in the flowchart and/or block diagram block or blocks.

Computer readable program instructions for carrying out operations of embodiments of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language, programming languages for machine learning or artificial intelligence such as C++, Python, Java, C, C#, Scala, CUDA or similar programming languages.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), ASICS, or programmable logic arrays (PLA) may execute the computer readable program instructions.

These computer readable program instructions may be provided to a processor of a computer, or other programmable data processing apparatus to produce a machine, such that the instructions, which execute via the processor of the computer or other programmable data processing apparatus, implement the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

Hardware and/or Software

Some of the functional components described in this specification have been labeled as systems or units in order to more particularly emphasize their implementation independence. A system or unit may be implemented as a hardware circuit (e.g., custom VLSI circuits or gate arrays, off-the-shelf semiconductors such as logic chips, transistors, or other discrete components, etc.) or in programmable hardware devices (e.g., field programmable gate arrays, programmable array logic, programmable logic devices, etc.). Alternatively, a system or unit may also be implemented in software for execution by various types of processors. For example, a system, unit or component of executable code may comprise one or more physical or logical blocks of computer instructions, which may be organized as an object, procedure, or function. The executables of an identified system or unit need not be physically located together, but may comprise disparate instructions stored in different locations which, when joined logically together, comprise the system or unit and achieve the stated purpose for the system, unit or component.

In general, a hardware element (e.g., CPU, GPU, RAM, ROM, etc.) may refer to any hardware structures arranged to perform certain operations. In one embodiment, for example, the hardware elements may include any analog or digital electrical or electronic elements fabricated on a substrate. The fabrication may be performed using silicon-based integrated circuit (IC) techniques, such as complementary metal oxide semiconductor (CMOS), bipolar, and bipolar CMOS (BiCMOS) techniques, for example. Examples of hardware elements may include processors, microprocessors, circuits, circuit elements (e.g., transistors, resistors, capacitors, inductors, and so forth), integrated circuits, application specific integrated circuits (ASICs), programmable logic devices (PLD), digital signal processors (DSP), field programmable gate array (FPGA), logic gates, registers, semiconductor devices, chips, microchips, chip sets, and so forth. However, the embodiments are not limited in this context.

Also noted above, some embodiments may be embodied in software. The software may be referenced as a software module or element. In general, a software element may refer to any software structures arranged to perform certain operations. In one embodiment, for example, the software elements may include program instructions and/or data adapted for execution by a hardware element, such as a processor. Program instructions may include an organized list of commands comprising words, values, or symbols arranged in a predetermined syntax that, when executed, may cause a processor to perform a corresponding set of operations.

A system or unit of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices and disparate memory devices.

Furthermore, systems/units may also be implemented as a combination of software and one or more hardware devices.

### General

The terminology used herein is for the purpose of describing particular embodiments only and is not intended to be limiting of the embodiments provided herein. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

Reference throughout this specification to “one embodiment,” “an embodiment,” “some embodiments”, or similar language means that a particular feature, structure, or characteristic described in connection with the embodiment is included in at least one embodiment provided herein and may refer to the same or different embodiments.

While the disclosure outlines exemplary embodiments, it will be appreciated that variations and modifications will occur to those skilled in the art. For example, although the illustrative embodiments are described herein as a series of acts or events, it will be appreciated that the present invention is not limited by the illustrated ordering of such acts or events unless specifically stated. Some acts may occur in different orders and/or concurrently with other acts or events apart from those illustrated and/or described herein, in accordance with the embodiments. For example, the functions noted in the blocks may occur out of the order noted in the Figures. For example, two blocks shown in succession may, in fact, be executed substantially concurrently, or the blocks may sometimes be executed in the reverse order, depending upon the functionality involved.

Aspects of the present techniques are described herein with reference to flowchart illustrations and/or block diagrams of methods, apparatus (systems), and computer program products. The flowchart and block diagrams in the Figures illustrate the architecture, functionality, and operation of possible implementations of systems, methods, and computer program products according to various embodiments of the present techniques. In this regard, each block in the flowchart or block diagrams may represent a module, segment, or portion of instructions, which comprises one or more executable instructions for implementing the specified logical function(s). It will be understood that each block of the flowchart illustrations and/or block diagrams, and combinations of blocks in the flowchart illustrations and/or block diagrams, can be implemented by computer readable program instructions. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Moreover, in particular regard to the various functions performed by the above described components (assemblies, devices, circuits, etc.), the terms used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments.

The embodiment was chosen and described in order to best explain the principles of the invention and the practical application, and to enable others of ordinary skill in the art to understand the invention for various embodiments with various modifications as are suited to the particular use contemplated.

A variety of other means and/or structures for performing the function and/or obtaining the results and/or one or more of the advantages described herein may be utilized, and each of such variations and/or modifications is deemed to be within the scope of the implementations described herein. More generally, all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific implementations described herein. It is, therefore, to be understood that the foregoing implementations are presented by way of example only and that, within the scope of the appended claims and equivalents thereto, implementations may be practiced otherwise than as specifically described and claimed. The description of the various embodiments herein has been presented for purposes of illustration and description, but is not intended to be exhaustive or limited to the invention in the form disclosed. It will be apparent to those skilled in the art that various modifications and variations can be made to the embodiments provided herein without departing from the spirit and scope of the invention.

Additional embodiments are provided herein. The following paragraphs provide a summary of various aspects of training and using an NLP system to predict biophysiochemical properties of an amino acid sequence using natural language processing (NLP). These embodiments are not to be limited to the following exemplary embodiments. In general, an embedding may refer to vector(s) representations of data.

### Additional Embodiments May Include:

### Training the NLP System

Clause 1. A computer-implemented method for training a protein language Natural Language Processing (NLP) system to predict binding affinity comprising:
in a first phase, training, using one or more processors, the protein language NLP system with at least one protein sequence dataset in a self-supervised manner; and
in a second phase, training, using one or more processors, the protein language NLP system, wherein the protein language NLP system comprises features from the first phase of training.

Clause 2. The computer-implemented method of Clause 1, wherein the first phase comprises:
training a first transformer based module with a first dataset comprising information obtained from TCR sequences in a self-supervised manner;
training a second transformer based module with a second dataset comprising information obtained from epitope sequences in a self-supervised manner;
providing the output of the first and second transformer models to a cross-attention module, wherein the cross-attention module computes cross attention, using one or more processors, between the outputs of the first transformer model and the outputs of the second transformer model; and
providing the output of the cross attention module to one or more second neural networks to determine binding probabilities between tuples, wherein each tuple includes an epitope and a TCR.

Clause 3. The method of Clause 1 or Clause 2, wherein the first dataset comprises a plurality of TCR sequences that have each undergone tokenization at an individual amino acid-level, an n-mer level, or a sub-word level.

Clause 4. The method of Clause 1 or Clause 2, wherein the second dataset comprises a plurality of epitope sequences that have each undergone tokenization at an individual amino acid-level, an n-mer level, or a sub-word level.

Clause 5. The method of any preceding clause, wherein about 10-20% of the amino acids in the first dataset are masked and wherein about 10-20% of the amino acids in the second dataset are masked.

Clause 6. The method of any preceding clause, wherein the first transformer model with self-attention further comprises a robustly optimized bidirectional encoder representations from transformers approach model, and the second transformer model with self-attention further comprises a robustly optimized bidirectional encoder representations from transformers approach model.

Clause 7. The method of any preceding clause, wherein the first and/or second dataset is normalized.

Clause 8. The method of any preceding clause, wherein the first transformer module comprises a first transformer model with self-attention and the second transformer model comprises a second transformer model with self-attention.

Clause 9. The method of any preceding clause, further comprising: training the first transformer until meeting a first criterion and the second transformer until meeting a second criterion.

Clause 10. The method of any preceding clause, wherein the first transformer model with self-attention is configured such that the input is added to the output via a skip connection, and/or the second transformer model with self-attention is configured such that the input is added to the output via a skip connection.

Clause 11. The method of any preceding clause, wherein the output of the cross attention module is fed forward to the second neural network through at least one feed forward layer.

Clause 12. The method of any preceding clause, wherein the output of the cross attention module comprises categorical features combined with representations of sequences to form concatenated representations of sequence and categorical feature embeddings that is provided as input to the second neural network.

Clause 13. The method of any preceding clause, wherein the second neural net is trained until meeting a specified criterion to generate binding probabilities for tuples.

Clause 14. The method of any of the preceding clauses, wherein the protein language NLP system may be trained to predict a binding affinity or a level thereof of a candidate TCR to an candidate epitope.

Clause 15. The method of any of the preceding clauses, the method further comprising: generating, for display on a display screen, information from a salience module that indicates a contribution of respective amino acids to the prediction of the binding affinity.

Clause 16. The method of any of the preceding clauses, wherein the predictive protein language NLP system may be further trained with experimental data to validate predicted binding affinities of tuples of epitopes and TCRs.

Clause 17. The method of any of the preceding clauses, wherein the length of the TCR sequence in the TCR sequence dataset is 9-11 amino acids, or preferably, 10 amino acids.

Clause 18. The method of any of the preceding clauses, wherein the TCR sequence is a CDR3 sequence.

Clause 19. The method of any of the preceding clauses, wherein the length of the epitope sequence is 7 to 9 amino acids, or preferably 8 amino acids.

Clause 20. The method of any of the preceding clauses, wherein the annotated dataset is modified to include tuples of TCR sequences and epitopes that are non-binders, and the second neural network is trained with the modified annotated dataset.

Clause 21. The method of any of the preceding clauses, wherein the candidate TCR and the candidate epitope tuple has not been previously used to train the protein language NLP system.

Clause 22. The method of any of the preceding clauses, further comprising determining whether a cysteine residue is present at the N-terminus of the TCR sequences and if the cysteine residue is not present, adding a cystine residue to the N-terminus.

Clause 23. The method of any of the preceding clauses, further comprising determining whether a phenylalanine residue is present at the C-terminus of the TCR sequences and if the phenylalanine residue is not present, adding a phenylalanine residue to the C-terminus.

Clause 24. The method of any of the preceding clauses, comprising determining whether a cysteine residue is present at the N-terminus of the candidate TCR sequence and if the cysteine residue is not present, adding a cystine residue to the N-terminus, and determining whether a phenylalanine residue is present at the C-terminus of the candidate TCR sequence and if the phenylalanine residue is not present, adding a phenylalanine residue to the C-terminus.

Clause 25. The method of any of the preceding clauses, comprising preprocessing the TCR sequence dataset by selecting for sequences with a specified HLA class.

Clause 26. The method of any of the preceding clauses, comprising categorizing the HLA sequences and filtering the dataset based on sequence size.

Clause 27 The method of any of the preceding clauses, comprising clustering the sequences and generating datasets for training.

Clause 28. The method of any of the preceding clauses, wherein the protein language NLP system is trained with short sequences (e.g., 8 to 11 amino acids) of both epitope sequences and TCR sequences to generate a prediction of binding affinity.

Clause 29. The method of any of the preceding clauses, wherein the protein language NLP system is trained on primary amino acids sequences absent structural information.

Clause 30. A computer-implemented method for preprocessing training data, comprising:
obtaining a dataset comprising TCR epitope pairs, wherein the TCR epitope pairs are deduplicated;
categorizing the TCR amino acid sequences into a plurality of categories;
filtering the dataset to retain the category of human HLA class I pairs;
capping TCR amino acid sequences with amino acid “C” at the N-terminus and with amino acid “F” at the C-terminus, if said amino acid residue is not present; and
filtering the dataset to retain amino acid sequences of a predetermined length.

Clause 31. A method of training a computer-implemented system comprising a protein language NLP system, comprising:
obtaining the dataset of claim 30;
splitting the dataset into a plurality of subsets, wherein the dataset is optionally clustered based on sequence identity;
tokenizing and masking the respective subsets;
training the protein language NLP system on one or more subsets, wherein the protein language NLP system comprises a first transformer to process TCR sequences and a second transformer to process epitope sequences, wherein training of the first and second transformer proceeds until meeting a specified respective threshold regarding predicting probabilities of amino acids at masked residue positions;
computing cross-attention between the output of the first transformer trained on TCR sequences and the second transformer trained on epitopes with a cross-attention module that receives the outputs of the first and second transformers; and providing as input embeddings from the output of the cross-attention module along with categorical variables (e.g., an HLA category) to a neural network, and training the neural network until meeting a second specified threshold for predicting binding affinity between tuples of TCRs and epitopes.

### Training an NLP System, Wherein First Phase of Training is Complete

Clause 1. A computer-implemented method for predicting binding affinities using natural language processing (NLP) comprising:
obtaining a protein language NLP system trained, using one or more processors, in a first phase on epitope sequence datasets and TCR sequence datasets in a self-supervised manner, wherein the first phase of training includes computing cross-attention between TCR embeddings and epitope embeddings using a cross attention module; and
generating concatenated representations of sequence and categorical feature embeddings based on output of the cross attention module; and
training, using one or more processors, a protein language NLP system comprising a second neural network with the concatenated representations to predict binding affinity between tuples of TCRs and epitopes.

Clause 2. The computer-implemented method according to clause 1, comprising:
training, using one or more processors, the protein language NLP system with an annotated TCR and epitope sequence dataset in a supervised manner to predict binding affinity between TCRs and epitopes.

Clause 3. The computer-implemented method for predicting binding affinities of Clause 1 or Clause 2, comprising:
predicting with the trained predictive protein language NLP system, the binding affinity between tuples of candidate TCRs and candidate epitopes.

### Trained NLP System

Clause 1. A computer-implemented method for predicting binding affinity using natural language processing (NLP) comprising:
providing a trained predictive protein language NLP system:
   trained, in a first phase using one or more processors, with a TCR sequence dataset and an epitope sequence dataset in a self-supervised manner, and wherein cross-attention was computed between TCR embeddings and epitope embeddings to improve predictions of binding affinity between tuples of TCRs and epitopes; and
   trained, in a second phase using one or more processors, with information obtained from the first phase;
   receiving an input query, from a user interface device coupled to the trained predictive protein language NLP system, comprising one or more candidate amino acid sequences; and
   generating, by the trained predictive protein language NLP system using one or more processors, an output comprising a prediction including binding affinities for the one or more candidate tuples.

Clause 2. The computer-implemented method of Clause 1 comprising:
Accessing, using one or more processors, the trained predictive protein language NLP system, trained in the first phase by masking at least a portion (e.g., about 10-20%) of individual amino acids in the TCR and/or epitope sequence datasets.

Clause 3. The computer-implemented method of Clause 1 or 2, wherein the TCR or epitope sequence dataset has undergone individual amino acid-level tokenization, n-mer level tokenization, or sub-word level tokenization of respective amino acid sequences.

Clause 4. The computer-implemented method of any preceding clause, wherein the trained protein language NLP system is generated by training, using one or more processors in a first phase, a first neural network and in a second phase a second neural network.

Clause 5. The computer-implemented method of any of Clauses 1 to 4, wherein the one or more first neural networks comprise a transformer model configured for self-attention.

Clause 6. The computer-implemented method of any of Clauses 1 to 5, wherein the one or more second neural networks comprise a perceptron.

Clause 7. The computer-implemented method of any of Clauses 1 to 6, wherein the transformer model configured for self-attention further comprises a robustly optimized bidirectional encoder representations from transformers model.

Clause 8. The computer-implemented method of any of Clauses 1 to 7, wherein the computer-implemented method further comprises: receiving a plurality of candidate TCR sequences and candidate epitope sequences generated in silico, generating using one or more processors, a prediction of a binding affinity or a level thereof for the candidate TCR sequences and candidate epitope sequences, and displaying the candidate TCR sequences and candidate epitope sequences according to a ranking of the predicted binding affinity or a level thereof for tuples of the candidate TCR sequences and candidate epitope sequences.

Clause 9. The computer-implemented method of any of Clauses 1 to 8, wherein the computer-implemented method further comprises: providing for display on a display screen information from a salience module that indicates a contribution of respective amino acids to a predicted binding affinity or a level thereof, such as a level of attention for each amino acid of the candidate TCR sequence and candidate epitope sequence.

Clause 11. The computer-implemented method of Clause 1, comprising:
providing a trained protein language NLP system comprising:
   a first neural network comprising a first transformer and a second transformer, the first transformer trained using one or more processors, in a first phase, with a first dataset comprising information obtained from TCR sequences in a self-supervised manner, and the second transformer module trained with a second dataset comprising information obtained from epitope sequences in a self-supervised manner; and
   a second neural network, trained using one or more processors in a second phase, with information obtained from the first neural network in the first phase of training;
receiving an input query, from a user interface device coupled to the trained protein language NLP system, comprising one or more candidate TCR sequences and candidate epitope sequences; and
generating using one or more processors, by the trained protein language NLP system, an output comprising a prediction of a binding affinity or a level thereof for the candidate TCR sequences and candidate epitope sequences.

Clause 13. The computer-implemented method of Clause 12, wherein the trained protein language NLP system is generated by providing the output of the first and second transformer based to a cross attention module, wherein the cross attention module computes, using one or more processors, cross attention between the output of the first transformer and the output of the second transformer using a first set of inputs and between the output of the second transformer and the output of the first transformer using a second set of inputs different from the first set; and
providing information from the output of the cross attention module to train, using one or more processors, a second neural network to determine binding probabilities between tuples, with each tuple including an epitope sequence and a TCR sequence.

Clause 14. The method of any of Clauses 11 to 13, wherein the first dataset comprises a plurality of TCR sequences that have each undergone tokenization at an individual amino acid-level, an n-mer level or a sub-word level.

Clause 15. The method of any of Clauses 11 to 14, wherein the first transformer model with self-attention further comprises a robustly optimized bidirectional encoder representations from transformers model, and the second transformer model with self-attention further comprises a robustly optimized bidirectional encoder representations from transformers model.

Clause 16. The method of any of Clauses 11 to 15, wherein about 10-20% of the amino acids in the first dataset are masked and about 10-20% of the amino acids in the second dataset are masked.

Clause 17. The method of any of Clauses 11 to 17, wherein the first transformer model comprises a first transformer model with self-attention and the second transformer model comprises a second transformer model with self-attention.

Clause 18. The method of any of Clauses 11 to 17, further comprising: training the first transformer using one or more processors, until meeting a first criterion and training the second transformer using one or more processors, until meeting a second criterion.

Clause 19. The method of any of Clauses 11 to 18, wherein the first transformer module with self-attention is configured such that the input is added to the output via a skip connection, and the second transformer module with self-attention is configured such that the input is added to the output via a skip connection.

Clause 20. The method of any of Clauses 11 to 20, wherein the output of the cross attention model is fed forward through at least one feed forward layer.

Clause 21. The method of any of Clauses 11 to 21, wherein categorical features obtained from the cross attention module are obtained and combined with representations of sequences to form concatenated representations of sequence and categorical feature embeddings for incorporation into the second neural network comprising a perceptron.

Clause 22. The method of any of Clauses 11 to 22, wherein the neural network is trained, using one or more processors, until meeting a specified criterion to generate binding probabilities for tuples of TCR sequences and epitopes sequences.

Clause 23. The method of any of the preceding clauses, the method further comprising: generating, for display on a display screen, information from a salience module that indicates a contribution of respective amino acids to the prediction of the binding affinity.

Clause 24. The method of any of the preceding clauses, wherein the predictive protein language NLP system may be further trained with experimental data to validate predicted binding affinities of a candidate TCR sequence and candidate epitope sequence.

Clause 25. A computer-implemented method for predicting binding affinities of an amino acid sequence using natural language processing (NLP) comprising:
obtaining a protein language NLP system trained, using one or more processors, in a first phase on a TCR sequence dataset and/or an epitope sequence dataset in a self-supervised manner; and
training, using one or more processors, the obtained protein language NLP system with an annotated TCR and epitope sequence dataset in a supervised manner to predict a binding affinity; and
using the trained protein language NLP system to predict a binding affinity.

Clause 26. The computer-implemented method of Clause 13 for predicting binding affinities or a level thereof using natural language processing (NLP) comprising:
obtaining embeddings from a cross attention module computing, using one or more processors, to compute cross attention between TCR embeddings and epitope embeddings; and
training, using one or more processors, a protein language NLP system comprising a neural net using the information from the first phase of training to predict binding affinity between tuples of TCRs and epitopes.

### Trained Executable NLP System

Clause 1. A computer-implemented method for predicting binding affinity or a level thereof of a TCR to an epitope using natural language processing (NLP) comprising:
receiving an executable program corresponding to a trained protein language NLP system:
   trained, in a first phase using one or more processors, with a TCR sequence dataset and a epitope sequence dataset in a self-supervised manner, and
   trained, in a second phase using one or more processors, with embeddings from the first phase;
loading the executable program into memory and executing with one or more processors the executable program corresponding to the trained protein language NLP system.

Clause 2. The computer-implemented method of Clause 1 further comprising:
receiving an input query, from a user interface device coupled to the executable program, comprising a candidate TCR sequence and a candidate epitope sequence;
generating using one or more processors, by the executable program, an output comprising a prediction including a binding affinity or a level thereof between a candidate TCR sequence and a candidate epitope sequence; and
displaying (optionally), the output on a display screen of a device, the predicted binding affinity or a level thereof between the candidate TCR sequence and the candidate epitope sequence.

Clause 3. The computer-implemented method of any of Clauses 1 or Clause 2 further comprising: receiving an executable program corresponding to the protein language NLP system, with the NLP system trained in the first phase by masking at least a portion of individual amino acids (e.g., about 10-20%) in the TCR sequence dataset and/or the epitope sequence dataset.

Clause 4. The computer-implemented method of any of Clauses 1 to 3 further comprising: receiving an executable program corresponding to the protein language NLP system, with the NLP system trained, using one or more processors, in the first phase using a TCR sequence dataset and/or an epitope sequence dataset that has undergone individual amino acid-level, n-mer level, or sub-word tokenization of respective protein sequences.

Clause 5. The computer-implemented method of any of Clauses 1 to 4 further comprising: receiving an executable program corresponding to a protein language NLP system, with the NLP system generated by training, using one or more processors, in a first phase a first neural network and in a second phase a second neural network comprising features from the first neural network.

Clause 6. The computer-implemented method of Clause 5, wherein the first neural network module comprises at least one transformer model with self-attention.

Clause 7. The computer-implemented method of Clause 5, wherein the first neural network comprises a first transformer with self-attention trained on a tokenized and masked TCR sequence dataset, and a second transformer with self-attention trained on a tokenized and masked epitope sequence dataset.

Clause 8. The computer-implemented method of Clause 7, wherein the output of the first and second transformers are provided to a cross-attention module, and the cross-attention module computes cross-attention, using one or more processors, between embeddings of the epitope sequence dataset and embeddings of the TCR sequence dataset.

Clause 9. The computer-implemented method of Clause 7 or 8, wherein the at least one transformer model with self-attention comprises a robustly optimized bidirectional encoder representations from transformers model.

Clause 10. The computer-implemented method of any of Clauses 1 to 9, further comprising a second neural network.

Clause 11. The computer-implemented method of Clause 10, wherein the second neural network comprises a perceptron.

Clause 12. The computer-implemented method of Clause 11, wherein concatenated representations of sequence and categorical feature embeddings from the first neural network are provided as input to the second neural network.

Clause 13. The computer-implemented method of any of Clauses 1 to 12, further comprising: receiving a plurality of candidate TCR sequences and candidate epitope sequences generated in silico, generating a prediction of a binding affinity or a level thereof between a candidate TCR sequence and a candidate epitope sequence; and optionally, displaying tuples of the candidate TCR sequences and candidate epitope sequences according to a ranking of the predicted binding affinity or a level thereof.

Clause 14. The computer-implemented method of any of Clauses 1 to 13, further comprising: providing for display on a display screen information from a salience module that indicates a contribution of respective amino acids to the prediction of the binding affinity or a level thereof between a candidate TCR sequence and a candidate epitope sequence.

Clause 15. The computer-implemented method of any of Clauses 1 to 14, further comprising: providing on the display screen information from a salience module that indicates a level of attention for each amino acid of a candidate TCR sequence and a candidate epitope sequence for a predicted binding affinity.

### System

Clause 1. A system or apparatus is provided for training a protein language NLP system comprising one or more processors to predict a binding affinity or a level thereof comprising one or more processors for executing instructions corresponding to a protein language NLP system to:
provide a protein language NLP system trained to predict a binding affinity or a level thereof of a TCR to an epitope;
receive an input query, from a user interface device coupled to the trained protein language NLP system, comprising a candidate TCR sequence and candidate epitope sequence;
generate, using one or more processors, by the trained protein language NLP system, a prediction including a binding affinity or a level thereof between the candidate TCR and the candidate epitope; and
display (optionally), on a display screen of a device, the predicted binding affinity or the level thereof between the candidate TCR and the candidate epitope.

Clause 2. The system or apparatus of Clause 1 comprising one or more processors for executing instructions corresponding to a protein language NLP system, the system:
trained using one or more processors, in a first phase, with a TCR sequence dataset and an epitope sequence dataset in a self-supervised manner, and
trained using one or more processors, in a second phase and including features from the first phase.

Clause 3. The system or apparatus according to Clause 1 or 2, further comprising: receiving an executable program corresponding to the protein language NLP system, with the NLP system trained in the first phase by masking at least a portion of individual amino acids (e.g., about 10-20%) in the TCR sequence dataset and/or the epitope sequence dataset.

Clause 4. The system or apparatus according to any of Clauses 1 to 4 further comprising: receiving an executable program corresponding to the protein language NLP system, with the NLP system trained in the first phase using a TCR sequence dataset and/or an epitope sequence dataset that has undergone individual amino acid-level, n-mer level, or sub-word tokenization of respective protein sequences.

Clause 5. The system or apparatus according to any of Clauses 1 to 4 further comprising: receiving an executable program corresponding to a protein language NLP system, with the NLP system generated by training in a first phase a first neural network, and in a second phase a second neural network comprising features from the first neural network.

Clause 6. The system or apparatus according to Clause 5, wherein the first neural network module comprises at least one transformer model with self-attention.

Clause 7. The system or apparatus according to Clause 5 or 6, wherein the first neural network module comprises a first transformer and a second transformer, wherein the first transformer with self-attention is trained, using one or more processors, on a tokenized and masked epitope sequence dataset, and wherein the second transformer with self-attention is trained, using one or more processors, on a tokenized and masked TCR sequence dataset.

Clause 8. The system or apparatus according to Clause 7, wherein the output of the first neural network is provided to a cross-attention module, and the cross-attention module computes cross attention, using one or more processors, between embeddings of the epitope sequence dataset and embeddings of the TCR sequence dataset.

Clause 9. The system or apparatus according to Clause 7 or 8, wherein the at least one transformer model with self-attention comprises a robustly optimized bidirectional encoder representations from transformers model.

Clause 10. The system or apparatus according to any of Clauses 1 to 9, further comprising a second neural network.

Clause 11. The system or apparatus according to Clause 10, wherein the second neural network comprises a perceptron.

Clause 12. The system or apparatus according to Clause 11, wherein concatenated representations of sequence and categorical feature embeddings derived from the first neural network are provided as input to the second neural network.

Clause 13. The system or apparatus according to any of Clauses 1 to 12, further comprising: receiving a plurality of candidate TCR and candidate epitope sequences generated in silico, generating using one or more processors, a prediction of binding affinity or a level thereof between the candidate TCR sequences and the candidate epitopes; and optionally, displaying on a display screen of a device a ranking of the candidate TCR sequences and the candidate epitopes based on a prediction of binding affinity or a level thereof.

Clause 14. The system or apparatus according to any of Clauses 1 to 13, further comprising: providing for display on a display screen information from a salience module that indicates a contribution of respective amino acids to the prediction of the one or more binding affinity or a level thereof between the candidate TCR sequences and the candidate epitopes.

Clause 15. The system or apparatus according to any of Clauses 1 to 14, further comprising: displaying on the display screen information from a salience module that indicates a level of attention for each amino acid of a respective tuple comprising a candidate TCR sequence and a candidate epitope.

Clause 16. The system or apparatus of any of the preceding clauses, wherein clustering of the datasets was performed based on sequence homology.

Clause 17. The system or apparatus of any of the preceding clauses, wherein the length of the TCR sequence in the TCR sequence dataset is 9-11 amino acids, or preferably, 10 amino acids.

Clause 18. The system or apparatus of any of the preceding clauses, wherein the TCR sequence is a CDR3 sequence.

Clause 19. The system or apparatus of any of the preceding clauses, wherein the length of the epitope sequence is 7 to 9 amino acids, or preferably 8 amino acids.

Clause 20. The system or apparatus of any of the preceding clauses, wherein the annotated dataset is modified to include tuples of TCR sequences and epitopes that are non-binders, and the second neural network is trained with the modified annotated dataset.

Clause 21. The system or apparatus of any of the preceding clauses, wherein the candidate TCR and the candidate epitope tuple has not been previously used to train the protein language NLP system.

Clause 22. The system or apparatus of any of the preceding clauses, further comprising determining whether a cysteine residue is present at the N-terminus of the TCR sequences and if the cysteine residue is not present, adding a cystine residue to the N-terminus.

Clause 23. The system or apparatus of any of the preceding clauses, further comprising determining whether a phenylalanine residue is present at the C-terminus of the TCR sequences and if the phenylalanine residue is not present, adding a phenylalanine residue to the C-terminus.

Clause 24. The system or apparatus of any of the preceding clauses, comprising determining whether a cysteine residue is present at the N-terminus of the candidate TCR sequence and if the cysteine residue is not present, adding a cystine residue to the N-terminus, and determining whether a phenylalanine residue is present at the C-terminus of the candidate TCR sequence and if the phenylalanine residue is not present, adding a phenylalanine residue to the C-terminus.

Clause 25. The system or apparatus of any of the preceding clauses, comprising preprocessing the TCR sequence dataset by selecting for sequences with a specified HLA class.

Clause 26. The system or apparatus of any of the preceding clauses, comprising categorizing the HLA sequences and filtering the dataset based on sequence size.

Clause 27 The system or apparatus of any of the preceding clauses, comprising clustering the sequences and generating datasets for training.

Clause 28. The system or apparatus of any of the preceding clauses, wherein the protein language NLP system is trained with short sequences (e.g., 8 to 11 amino acids) of both epitope sequences and TCR sequences to generate a prediction of binding affinity.

Clause 29. The system or apparatus of any of the preceding clauses, wherein the protein language NLP system is trained on primary amino acids sequences absent structural information and trained.

### Computer Readable Media

Clause 1. A computer program product comprising a computer readable storage medium having instructions for training a protein language NLP system to predict a binding affinity or a level thereof embodied therewith, the instructions executable by one or more processors to cause the processors to train the protein language NLP system to predict a binding affinity or a level thereof of a TCR to an epitope according to the methods provided herein.

Clause 2. The computer program product of Clause 1, wherein the computer program product comprises a computer readable storage medium having instructions corresponding to a protein language NLP system embodied therewith, the instructions executable by one or more processors to cause the processors to predict a binding affinity or a level thereof of a TCR to an epitope according to the methods provided herein.

Clause 3. The computer program product of Clause 1 or 2, wherein the computer-readable storage medium is provided having stored thereon the computer program product for predicting a binding affinity or a level thereof of a TCR to an epitope, according to any of the methods or systems provided herein.

Clause 4. A computer-readable data carrier is provided having stored thereon the computer program product for predicting a binding affinity or a level thereof of a TCR to an epitope, according to any of the methods or systems provided herein.

The summary is not intended to restrict the disclosure to the aforementioned embodiments. Other aspects and iterations of the disclosure are provided below.

### Examples

### Advantages

For machine learning approaches to be successful, systems are often trained with large volumes of annotated data, which can be time-consuming to generate. Additionally, traditional one-shot approaches may adjust parameters and iteratively retrain systems with the entire dataset until meeting specified criteria.

In contrast, present approaches utilize a different approach relying on the primary structure of proteins. By training a first neural network on specific sets of protein sequences, the neural network learns rules associated with the ordering of amino acids in TCR sequences and epitope sequences. This information is transferred to another neural network, which is fine-tuned with a compact, annotated dataset to predict binding affinities or levels thereof between an epitope sequence and a TCR sequence.

Present approaches provide a variety of technical improvements to the field of machine learning and advance the application of machine learning to biological systems. For example, present approaches accelerate the development and customization of machine learning systems to particular applications. By training a first neural network, the machine learning system may be further fine-tuned in a second training phase to improve prediction of binding affinities. Subsequent rounds of fine-tuning may also be performed rapidly as additional experimental data becomes available. Thus, present techniques provide a robust and rapid approach to developing and applying machine learning systems to predict binding affinity.

Further, present approaches greatly reduce the amount of annotated data needed to train a machine learning application for predicting binding affinities or levels thereof between TCR sequences and epitope sequences. Traditionally, for machine learning approaches to be successful, a sufficient amount of data is needed to train the system to meet a specified performance criteria. Here, the protein language NLP system uses primary amino acid sequences obtained from publicly available databases to learn rules of epitopes and TCR sequences, which allows the system to be trained with a smaller compact annotated dataset in the second phase of training. It is surprising that information learned purely based on the amino acid sequence order of proteins in a first phase of training is able to reduce the amount of annotated data that would otherwise be needed to train a machine learning system to predict TCR-epitope binding/binding affinity, in view of the lengths of TCR sequences and epitopes. Improved processing techniques (e.g., application of cross-attention) and improved preprocessing techniques demonstrated surprising improvements in accuracy of predictions as discussed below.

In aspects, a programming environment compatible with NLP techniques may be utilized, and may comprise or be integrated with one or more libraries of various types of neural network models. Database downloads of amino acid sequences may be obtained, and imported into the programming environment.

### Examples

The protein language NLP system may be trained and fine-tuned for predicting T-cell receptor-epitope binding specificity and prediction of TCR-epitope binding affinity for human MHC class I restricted epitopes by training with publicly available databases (e.g., VDJDB and EIDB). This system may be used to improve vaccine design and synthesis, etc. Representative examples are provided below. The examples provided herein are not intended to be limiting.

### Example Architecture

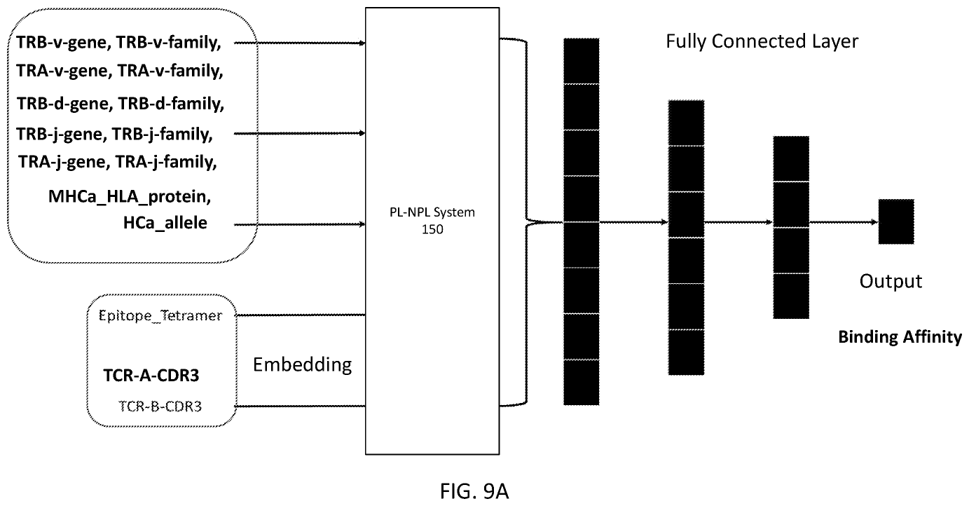

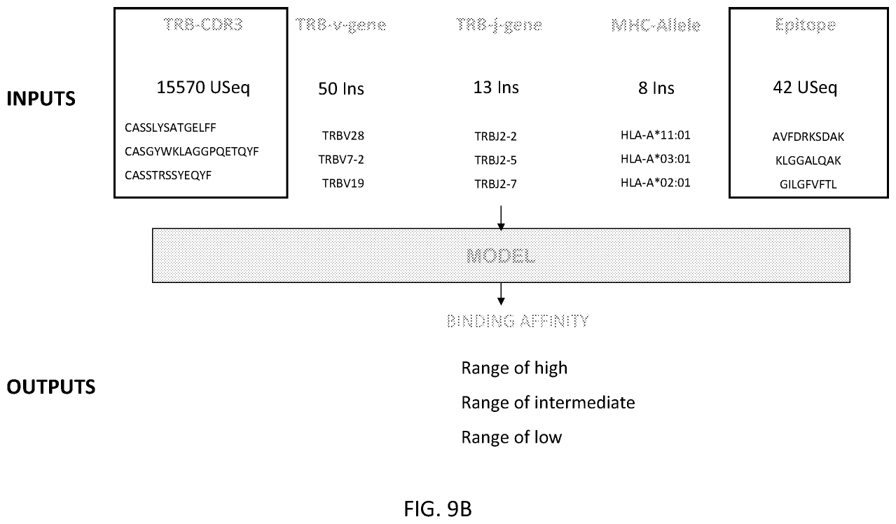

FIG. 9A is an example high-level architecture of the protein language NLP system used for predicting TCR-epitope binding. In this example, various inputs (e.g., TRA-v-gene, TRA-v-family, TRB-v-gene, TRB-v-family, TRB-d-gene, TRB-d-family, TRA-j-gene, TRA-j-family, TRB-j-gene, TRB-j-family, TRA-CDR3 (also referred to as TCR-A-CDR3), TRB-CDR3 (also referred to as TCR-B-CDR3), MHCa_HLA_protein and HCa_allelle, and epitope tetramer) are provided to fully connected layers of a neural network. In aspects, the following are treated as categorical variables: TRA-v-gene, TRA-v-family, TRB-v-gene, TRB-v-family, TRB-d-gene, TRB-d-family, TRA-j-gene, TRA-j-family, TRB-j-gene, TRB-j-family, MHCa_HLA_protein, and HCa_allele. In aspects, the following are treated as embedded variables (by tokenizing on an individual amino acid level): epitope, TCR-A-CDR3, and TCR-B-CDR3. The output is a predicted binding affinity. With present approaches, inputs such as TRA related sequences may easily be included. Predictions may be validated in the wet lab.

FIG. 9B shows results of TCR-binding prediction classification in accordance with certain aspects of the present disclosure. The top portion of the figure shows a prediction of binding or not binding, while the bottom portion of the figure shows a degree of predicted binding affinity (e.g., strongly specific, medium specific, weakly specific) for a given epitope. The protein language NLP system may classify epitopes into categories such as strongly specific, medium specific, or weakly specific binding affinity based on different threshold cutoff values or ranges. In this example, a TCR-epitope binding affinity was predicted for a given TCR-epitope combination, and example values of binding affinity using regression approaches are provided which fall within respective categories of binding affinity (strongly specific, medium specific, weakly specific).

FIG. 9B shows results of TCR-binding affinity predictions in accordance with certain aspects of the present disclosure. A list of epitopes was provided to the protein language NLP system and binding affinities were predicted for each epitope. In this example, various training sequences (TRB-CDR3, TRB-v-gene, TRB-j-gene, MHC alleles) along with candidate epitopes were provided to the system. The trained system provided as output, predicted binding affinities for the candidate epitopes.

The protein language NLP system, comprising a (TCR-Epitope) classification module, predicted the cognate epitopes of a given TCR from an exhaustive list of published epitopes based on human MHC class I restricted epitopes from publicly available databases (e.g., VDJDB and EIDB). Once classified, the protein sequence software further predicted the binding affinity of a given pair of TCR-epitope sequences (e.g., based on TCR-Epitope Regression techniques).

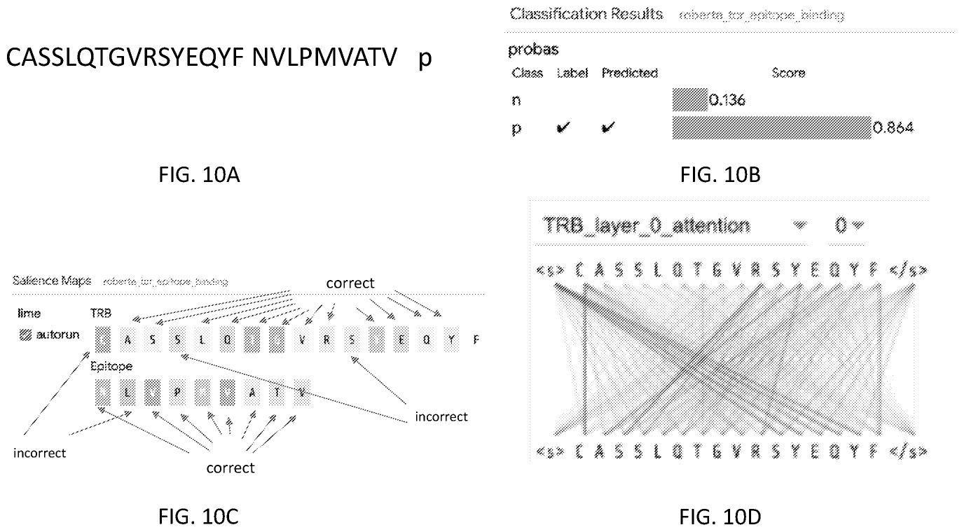

FIGS. 10A-10D show various aspects of user interfaces suitable for displaying and interpreting results generated by the protein language NLP system. A historical dataset may be used to fine-tune the protein language NLP system. In FIG. 10A, a user may enter novel candidate sequences (e.g., TRB and epitope sequences), previously unknown to the protein language NLP system, and generate binding predictions that appear in FIG. 10B. In FIG. the system's interpretation of which amino acids are important for binding are displayed. Here, a color coding scale/grayscale or other visualization technique may show strength of interaction, representing amino acids from least contributing to highly contributing.

FIG. 10B shows a view of classification results for epitopes, for example, whether an epitope binds to a TCR. FIG. 10C shows a view of a salience module showing a map of epitope-TCR binding. In this example, the TRB portion of the TCR is shown along with an epitope predicted to bind to the TRB portion. Individual amino acid residues may be color coded or shaded (grayscale) to illustrate the contribution of each amino acid to binding between the TCR and epitope. In this example, residues contributing to binding are shown as correct, and residues not contributing to binding are shown as incorrect. This feature allows insight into the predictions made by the system, and allows ease of fine-tuning epitopes to improve binding prediction scores. FIG. 10D shows an attention layer or connectivity between individual amino acid residues.

The output of the protein language NLP system may be provided in any suitable manner, including visually on a display, by an audio device, etc.

### EXAMPLE IMPLEMENTATION

### Example 1

Datasets were downloaded from publicly available resources. These datasets were tokenized and masked according to the techniques provided herein (e.g., individual amino acid tokenization with about 15% masking of individual amino acids of a dataset). This data was utilized to a train a protein language NLP system.

In aspects, a library of transformer models compatible with Python/PyTorch was obtained. In other aspects, transformer models may be developed.

In aspects, a first neural network, such as a robustly optimized bidirectional encoder representation with one or more transformers (RoBERTa) model was trained in a machine learning environment/platform such as Python/PyTorch. In aspects, a four GPU computing system with at least 500 GB RAM was utilized to train the neural network.

During the first phase of training, a RoBERTa model pretraining on UniRef50 (˜37.5M sequences) was used. The following configuration/setup was applied.

Tokenisation: vocabulary size of 32 (character-level IUPAC amino acid codes+special tokens) Architecture (˜38.6M parameters): RoBERTa transformer with: Number of layers: 12 layers; Hidden size: 512; Intermediate size: 2048; Attention heads: 8; Attention dropout: 0.1; Hidden activation: GELU; Hidden dropout: 0.1; Max sequence length: 512.

Self-supervised training (distributed across 8 GPUs with mixed-precision fp16 enabled): Effective batch size: 512 (64 times 8 GPUs); Optimizer: AdamW; Adam epsilon: 1e-6; Adam (beta_1, beta_2): (0.9, 0.99); Gradient clip: 1.0; Gradient accumulation steps: 2; Weight decay: 1e-2; Learning rate: 2e-4; Learning rate scheduler: linear decay with warmup of 1000 steps; Max training steps: 250 k (˜7 epochs).

The system may be trained in a second phase of training to be customized to TCR epitope binding (classification) or TCR epitope binding affinity (regression). Data sets including VDJdb, IEDB, McPAS-TCR, and PIRD may be obtained.

Input parameters (various combinations) included:
TCR-B-CDR3 (CDR3b), epitope; or
TCR-B-CDR3 (CDR3b), epitope, MHCa_HLA_protein and HCa_allelle; or
TCR-B-CDR3 (CDR3b), epitope, MHCa_HLA_protein and HCa_allelle, TRB-j-gene, TRB-j-family, TRB-v-gene, TRB-v-family; or
TCR-A-CDR3 (CDR3a), TCR-B-CDR3 (CDR3b), epitope; or
TCR-A-CDR3 (CDR3a), TCR-B-CDR3 (CDR3b), epitope, MHCa_HLA_protein and HCa_allelle.

In aspects, the input parameters included TCR-A-CDR3 (CDR3a), TCR-B-CDR3 (CDR3b), epitope, MHCa_HLA_protein and HCa_allelle, TRB-v-gene, TRB-v-family, TRB-j-gene, TRB-j-family, TRA-v-gene, TRA-v-family, TRA-j-gene, TRA-j-family, TRB-d-gene, TRB-d-family

In aspects, for classification (prediction of binding), a RoBERTa based model for TCR-epitope binding classification was used. The following setup/configuration was applied:

Tokenisation: vocabulary size of 32 (character-level IUPAC amino acid codes+special tokens).

Architecture: RoBERTa transformer as sequence processing unit (see above) with a final multi-layer perceptron (MLP) as classification head on top of concatenated sequence and categorical embeddings.

Supervised training: Effective batch size: 128; Optimizer: AdamW; Adam epsilon: 1e-8; Adam (beta_1, beta_2): (0.9, 0.99); Gradient clip: 1.0; Weight decay: 1e-4; Learning rate: 8e-5; Learning rate scheduler: linear decay with warmup of 600 steps; Number of epochs: 12.

In aspects, for regression analysis (a degree of binding), a RoBERTa based model for TCR-epitope regression was used. The following setup/configuration was applied.

Tokenisation: vocabulary size of 32 (character-level IUPAC amino acid codes+special tokens).

Architecture: RoBERTa transformer as sequence processing unit (see above) with a final multi-layer perceptron (MLP) as regression head on top of concatenated sequence and categorical embeddings.

Supervised training (hyperparameters): Effective batch size: 64; Optimizer: AdamW; Adam epsilon: 1e-8; Adam (beta_1, beta_2): (0.9, 0.99); Gradient clip: 1.0; Weight decay: 1e-2; Learning rate: 5e-5; Learning rate scheduler: linear decay; Number of epochs: 4.

Training at any phase proceeded until meeting a specified AUC criteria, for example, greater than 0.65; 0.70; 0.75; 0.80; 0.85; 0.90; 0.95; etc.

Present techniques may also be used for predicting HLA-peptide binding. In other aspects, present techniques may be applied to predict binding of pathogen proteins or peptides to human proteins (e.g., TCRs).

### Example 2

T-cell receptor (TCR) interactions with cognate epitopes are central to the adaptive immune system's response to antigens. Accurate prediction of TCR-epitope binding could potentially speed up the development of new therapeutic and preventative strategies for infectious, autoimmune, and chronic diseases. To accomplish this through the use of in-silico methods, by training a protein language model for TCRs, protein language models to predict binding between TCR and epitope sequences were implemented. The pretraining of the protein language model was accomplished using a large set of TCR sequences and this model was then fine-tuned for the downstream task of predicting TCR-epitope bindings across multiple HLA class-I backgrounds. Performance was evaluated on a balanced set of binders and non-binders for each epitope, minimizing any possible model shortcuts, such as HLA types. Pan-HLA versus HLA-specific models were compared and the effects were studied on the predictions of the number of TCRs per epitope as well as the sequence similarities between train and test splits. The results indicated that in-silico prediction of binding probability between unseen/novel epitopes and TCRs is achievable. It was demonstrated that protein language model embeddings, which are representations of amino acids in the context of a neural network (e.g., in some cases, embeddings may be low dimensional learned representations of variables in a transformer network), are better suited than BLOSUM and hand-crafted based embeddings. Finally, predictions are interpretated using a LIME framework.

According to the techniques provided herein, TCR-epitope binding predictions are solvable, and reasonable performance can be obtained when predicting binding for unseen CDR3β TCR sequences to epitopes in the training set.

Based on natural language processing (NLP), a new paradigm for modeling sequences through contextualized embedding (e.g., pretrained masked language models) is proposed herein. By treating amino acid sequences as characters in a language, self-supervised language models for TCRs and for epitopes were each trained on predicting masked characters in a large corpus of protein sequences. The pretrained model was then finetuned on downstream prediction tasks with limited amounts of labeled data.

A protein language model for TCR sequences has been developed to model TCR-epitope binding and to predict the binding probability between a given TCR and epitope. This model predicted binding between TCR's CDR3β sequences and HLA class I epitopes and examined the value of developing HLA-specific models. The model also evaluated the effect of TCR and epitope representability on model performance within the training set, as well as generalization to previously unseen TCRs and epitopes. Further, benchmarking was performed of the protein language model-based embeddings against other alternatives, such as BLOSUM and hand-crafted amino-acid physicochemical embeddings as implemented by Titan and ImRex models, respectively. Lastly, the model predictions are interpretable using the LIME framework, which allowed comparison between the predicted interactions with resolved 3D structure of pHLA-TCR complex.

Material and Methods

Data Preprocessing

FIGS. 19A and 19B show a flowchart of operations for preprocessing data. At operation 3100, published human TCR-epitope pairs were obtained from VDJdb, IEDB, McPAS-TCR and/or PIRD databases, and were combined and filtered for human HLA class I to generate a dataset²⁴⁻²⁷.

At operation 3110, the dataset was cleaned by adding N and C terminal caps, e.g., to sequences not having said caps. For example, CDR3β sequences should have conserved “C” and “F” amino acids at N- and C-terminals respectfully. The dataset was modified by explicitly adding the caps, e.g., if not already present.

At operation 3120, each HLA sequence or dataset was standardized to an HLA category. HLA categories included, for example, those designated by the WHO or other suitable organizations or entities.

At operation 3130, the dataset was filtered based on a minimum length of the HLA sequence, such as a length of 8 for epitopes and a length of 10 for CDR3β.

At operation 3140, duplicate sequences were removed from the dataset. Samples with non-standard amino acids and/or an undetermined HLA class were also removed. Samples duplicated between different data sources were also identified and removed.

In some aspects, e.g., optionally prior to clustering, negative sample generation was performed by randomly pairing TCR sequences (CDR3s) with non-binding epitope-MHC complexes. At operation 3150, non-binding candidates (TCRs) were selected for an epitope. At operation 3160, negative samples (optionally) were created by replacing the binding TCRs with randomly sampled non-binding candidates to achieve a 1:1 target ratio for each epitope, while keeping the HLA constraint. The integrity of pairing between MHC and epitope was maintained.

At operation 3170, clustering was performed on the dataset. TCR sequences were clustered using ting28 under default settings. Epitope sequences were clustered using IEDB (e.g., ImmunomeBrowser^{28,29}) clustering tool 25 with 70% minimum sequence identity threshold and the recommended clustering method: cluster-break for clear representative sequence.

At operation 3180, and to account for the epitope imbalance, some datasets were downsampled by imposing a limit on the number of TCRs per epitope.

At operation 3190, the dataset was split into subsets for training and benchmarking. For example, datasets were divided into subsets for training and validation. Three distinct versions of the dataset were created based on (1) random assignment of TCR, (2) TCR assignment based on ting clusters, and (3) epitope clustering-based assignment while ensuring that no epitope clusters were shared between train/test sets. For cross-validation purposes, the dataset was divided into five-folds for each of these versions.

In the latter case, it was ensured that no epitope clusters were shared between train test sets. For cross-validation purposes, the dataset was divided into fivefolds for each of these versions. This distinction in information overlaps between the three versions enabled a more refined assessment of the model's generalizability.
Random splits: 5-fold cv such that unique CDR3β sequences are randomly split between folds
Ting TCR clusters-based splits: 5-fold cv such that TCR clusters were split independently between folds i.e., a given cluster can only be bucketed in one of the folds.
Epitope-clusters based splits: 5-fold cv such that epitope clusters are split independently between folds.

### Models

Titan: 3 architectures were trained using TCR CDR3β and epitope sequences. The configuration was downloaded from IBM.box.com/v/titan_dataset.

Architecture 0—Inference: trained and published model from IBM.box.com/v/titan_dataset, where TCR CDR3β amino-acids are encoded as amino-acids and embedded using BLOSUM, and epitope sequences are encoded using SMILES and the embedding were learned.

Architecture 1—Titan retrained: TCR CDR3β are encoded as amino-acids and embedded using BLOSUM and epitope sequences were encoded using SMILES and the embeddings were learned.

Architecture 2—Titan finetuned: finetuning the model provided in titan_dataset that was trained by the authors on full TCR proteins encoded as AA and embedded using BLOSUM, and epitopes encoded as SMILES and embeddings were learned. TCR CDR3β amino-acids and embeddings using BLOSUM were used and epitopes using SMILES and embeddings were learned. The number of epochs was set to 20.

Architecture 3—Titan retrained: TCR CDR3β and epitopes were encoded as AA and embeddings were learned.

To split amino acid sequences, character-level tokenization with vocabulary corresponding to the IUPAC amino acid codes was used. For learning the probability distribution over amino acids in each sequence, a transformer-based RoBERTa type language model (LM) architecture was used, which was pre-trained in a self-supervised manner using a large corpus of TCR CDR3β sequences.

Self- (e.g., RoBERTa LM) and cross-attention mechanisms were applied, which takes as inputs tuples of epitope and TCR sequences to embed them individually using the pretrained protein language model. A downstream cross-attention module comprised six layers, each of which is processed in the following manner After normalizing the epitope and TCR sequences separately in a first step, the epitope and TCR sequences are passed through a multi-head attention model to compute self-attention. For the self-attention algorithm, the input is also added to the output via a skip connection to improve training, thereby summing the results with the original input.

In the second step, cross attention was computed between the epitope and the TCR, as well as between the TCR and the epitope. Finally, two feed forward layers were used to pass epitope and TCR sequences to a neural net. Accordingly, processing included self-attention, cross-attention and feed forward operations. Categorical features were converted independently into learnable embeddings. Together, the concatenated representations of sequence and categorical feature embeddings were appended and provided to a neural network.

Thereafter, the protein language model was fine-tuned for the downstream task of predicting binding between TCR and HLA class I epitope sequences. The embedding associated with the <CLS> token was extracted and sent to the fully connected neural network in order to predict the probability of binding between TCR-Epitope sequences (see, FIG. 2B). Additionally, the added value of additional categorical features such as HLA alleles was evaluated. Together, the concatenated representations of sequence and categorical feature embeddings were appended to a multilayer perceptron and trained end-to-end to generate binding probabilities for a given TCR and epitope sequence.

Benchmarking

ImRex and Titan models were trained on the same dataset of TCR (CDR3) and epitope sequences that were used to train the model. These comparisons used the same fivefold cross-validation splits and three categories of assignment as described herein for the model: random, TCR clusters, and epitope clusters. The average ROC-AUC and 95 percent confidence interval obtained after evaluating the test splits as an evaluation metric were used.

ImRex:

Model: Architecture 0—a model trained on VDjdb (august 2019 release).

Hyper parameter changes: batch size 32->128, dropout_conv: 0.25->0.1; max_length_epitope 11->45; max_length_CDR3β 20->40; lr 0.0001->0.00015; regularization 0.1->0.008; number of train epochs:20. Parameters were identified by doing hyperparameter search on one of the data splits.

LIME and Interpretability

The LIME algorithm was used to determine the importance of each amino acid in the protein³². To interpret the significance of the amino acids involved, a given input sequence was subjected to a masking process in which a fixed-size local dataset was created by generating “random” perturbations from the given sequence, and then a Ridge regression linear model was trained on the resulting dataset to generate scores that were then normalized with L-norm to generate the final scores Amino acids with positive scores support the prediction, whereas amino acids with negative scores contradict the prediction. This means that positively scored amino acids are likely to be the primary drivers of binding.

The Protein Data Bank (PDB) was used to download crystal structures with PDB IDs³³. To determine the interaction between amino acids for a chosen pair of CDR3B and epitope sequences, the residue distance matrix using the PyMOL tool³⁴ was calculated. The computed distances from maximum to minimum were depicted. The strongly interacting amino acids between CDR3B and epitope were visually examined using PyMOL computed hydrogen bond interactions.

Results

A self-supervised masked language model was trained on CDR3β sequences by tokenizing the sequences at the character level using a tokenizer with a vocabulary of IUPAC amino acid codes^{12,31}. For self-supervised model training, a random subset of tokens from each sequence was chosen as target labels for the model to predict. Across layers, all other tokens were implicitly considered contextual input. The transformer model processed token indices using self-attention and feed-forward modules after converting them to a sum of token and positional vector embeddings. The transformer model generates a probability distribution over the token vocabulary for each target token, with the final contextualized token embeddings serving as the prediction head (FIG. 2B). To train the transformer model, the entropy loss between predictions and target labels was optimized. Findings are summarized in the following sections.

The Protein Language Model Requires Only TCR and Epitope Sequences to Capture the TCR-Epitope Binding Prediction Signal

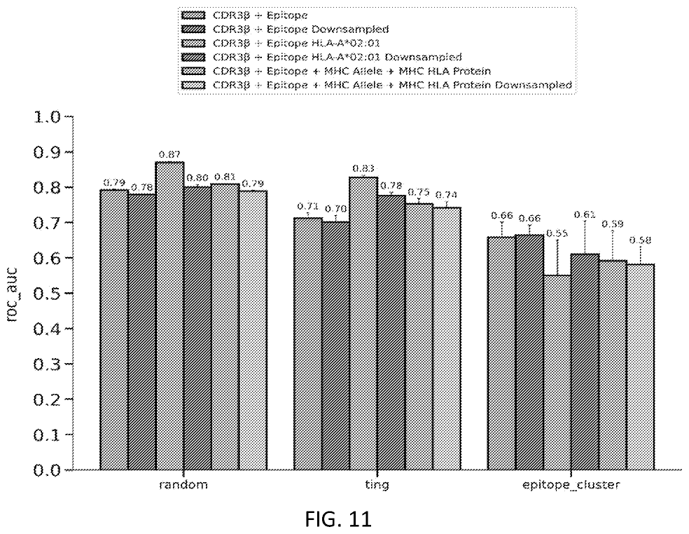

The protein language model was fine-tuned end-to-end using CDR3β and epitope amino acid sequences as input (TCR CDR3β+epitope pairs). Each training and testing split received an equal number of TCR sequences. The model achieved a ROC-AUC value of 0.79 for prediction in this design (FIG. 11, random assignment). However, random assignment of CDR3β sequences might result in information leakage due to the presence of closely related sequences in the training and testing sets. This is especially true for TCRs, which may differ in sequence but share important epitope binding motifs. To eliminate this bias, an approach that clusters TCRs based on the sequence similarity using the ting algorithm²⁸ was pursued. Following that, the TCR clusters were again divided into training and test sets. In this design, the ROC-AUC value decreased to 0.71 (FIG. 11, ting assignment), implying that there was indeed some information shared between train and test sets which might have slightly influenced the performance obtained on random splits. Overall, these findings demonstrate that training protein language models (TCR models) exclusively on CDR3β and epitope sequences was indeed sufficient to capture the signal for TCR-epitope binding prediction to a large extent.

The Predictions Made by the Protein Language Model were Robust to the Inherent Skewness of the TCR-Epitope Training Data Sets

The ideal data distribution for training any machine learning classification model is a balanced class distribution, as an imbalanced class distribution would influence the overall performance metrics, especially Accuracy and ROC-AUC. The effect of data skewness in the data set was determined, which may be caused by the presence of certain epitopes that have a greater number of corresponding TCR instances than the majority. In the present datasets, there was a difference in the number of CDR3B (>10000) and epitope (<1000) sequences. The approach was to down-sample samples from the majority classes (epitopes). After down-sampling TCRs from the most abundant epitopes, the ROC-AUC values decreased by 1% in both the random assignment and ting cluster assignment designs (0.79 vs 0.78, and 0.71 vs 0.70, FIG. 11). This change in performance could be due to a decrease in model overfitting on the most abundant epitope or simply due to a decrease in the number of classes for which the model performed optimally, resulting in a decrease in overall averaged performance. Nonetheless, in line with Moris et al., the over-representation of certain epitopes had no discernible effect on model performance¹⁹.

Adding HLA Type Information to the Model Slightly Increased the Performance

In this experiment, the effect of adding HLA typing information as covariates to the TCR and epitope sequences was studied. The addition of HLA as a categorical variable (HLA group and protein) to the basic model helped to achieve a marginal increase of ˜2% for random splits (0.81 vs 0.79, FIG. 11) and an increase of ˜4% for ting splits (0.75 vs 0.71, FIG. 11). This improvement in performance demonstrates that including HLA information in the model added value. Additionally, similar improvements were observed when models were trained with down sampled epitopes to reduce data skewness; 0.79 vs 0.78 and 0.74 vs 0.70, respectively, for random splits and ting-based splits.

Training the Protein Language Model for Specific HLA Improved Performance

The combined dataset used to train the protein language models discussed above falls in the category of pan-HLA models, i.e., it contained many different HLA types. Hence, in this experiment, the effect of restricting the task of predicting binding between TCR and epitope for only one type of HLA was investigated. In some examples, TCR-epitopes were restricted to those associated with HLA-A*02:01, the most prevalent HLA type in human populations.

This model achieved the highest ROC-AUCs: 0.87 and 0.83 in random assignments and ting-based TCR assignments, respectively (FIG. 11). Downsampling the epitopes had a similar effect on the performance of this HLA-A*02:01 specific model as well, with a reduction of 7% (0.80 vs 0.87) in the random assignment design and 5% (0.78 vs 0.83) in the ting clusters assignment. These findings indicate that while HLA-specific models can outperform pan-HLA models, they are more susceptible to data skewness than pan-HLA models.

The Number of TCR Instances Per Epitope Needed to Train Protein Language Model is Variable

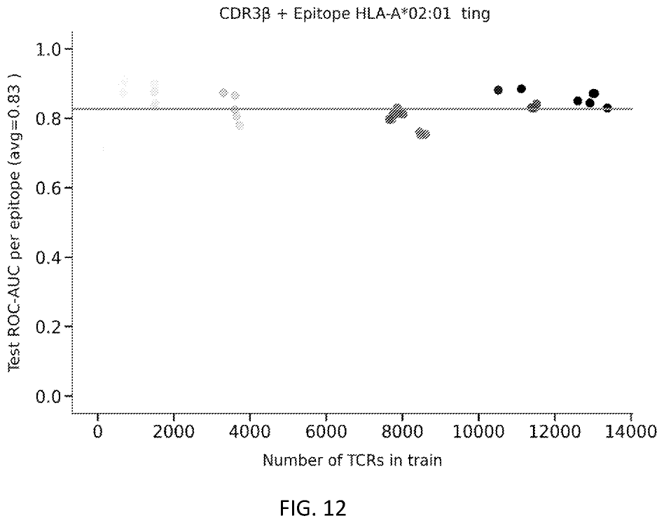

Here, the effect of the number of TCRs per epitope on the model's performance (FIG. 12) was examined. For simplicity, we examined this effect only using the HLA-A*02:01 specific model. It was discovered that when the number of TCR instances were small (less than 221), the model's performance was highly variable. At this level, ROC-AUC values were observed that were both greater than and less than the mean (0.83). With a larger number of TCR instances (greater than 221), the model's performance approached or exceeded the average. A possible explanation for these observations was that the larger the set of TCRs for a given epitope, the greater the likelihood of capturing a TCR-epitope binding signal (i.e., amino-acid motifs), whereas with smaller sets of TCRs, the presence of such signal is dependent on the composition and diversity of the TCR set.

Generalization to Previously Unseen Epitopes is Possible but More Difficult Due to the Training Data Set's Limited Diversity in the Epitope Space

Here, we sought to determine whether the models can generalize to previously unseen epitopes, a more difficult but highly desirable feature. First, epitope sequences were clustered, then assigned to test and train sets in a 5-cross-validation framework. We were mindful to ensure that epitope clusters in the test set were not present in the train set. Here, the model trained on CDR3β and epitope sequences only, achieved an average ROC-AUC of 0.66 before and after down sampling (FIG. 11). The model performances did not appreciably ameliorate upon addition of HLA covariates with or without down-sampling (ROC-AUC: 0.59 and 0.58). Further, restricting the model to a specific HLA (HLA-A*02:01) also did not improve the results (ROC-AUC: 0.55 and 0.61).

As a next step, the effect of similarity between epitope sequences in the training and test sets on the model performance was investigated. The edit distance between pairs of epitope sequences in the two sets was used to quantify this similarity. FIG. 13 demonstrates how highly variable the ROC-AUC metrics were for any given edit distance. Additionally, the performances were unrelated to the number of TCR instances per epitope, as both highly and poorly represented epitopes demonstrated above or below average performance. As a result, no clear pattern could be established for predicting the effect of epitope sequence similarity between the test and training sets.

Overall, these findings indicate that generalization to previously unknown epitopes is possible, but that a improved performance may be hampered by the low diversity of the epitope space in current training datasets.

The Protein Language Models Performed Better than n-Gram Based Classical ML Approaches

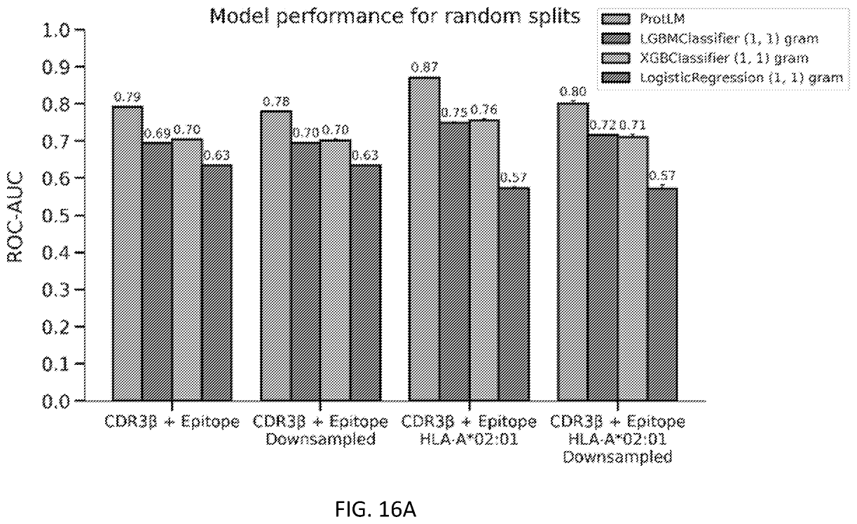

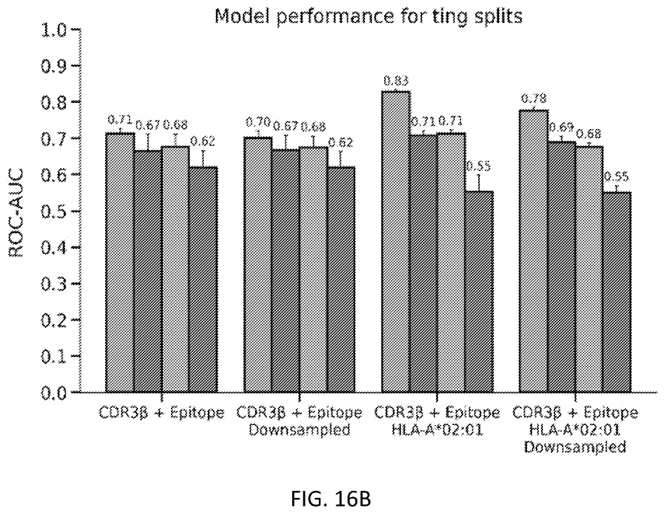

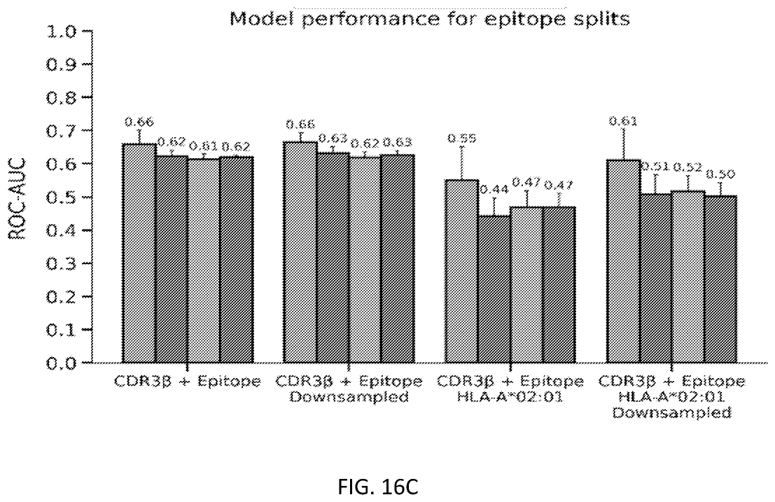

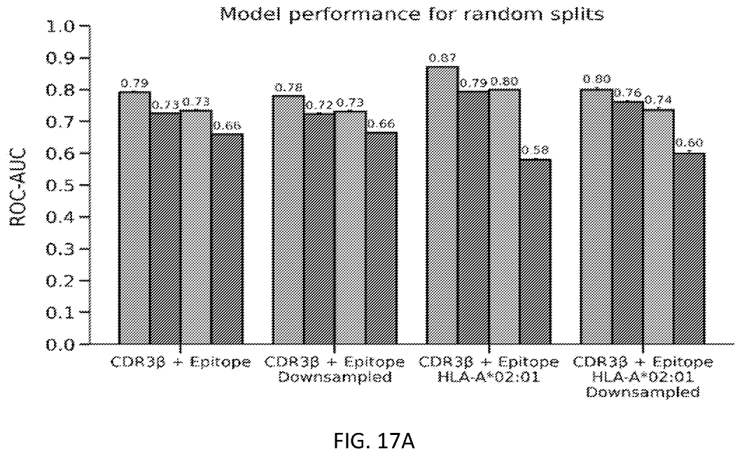

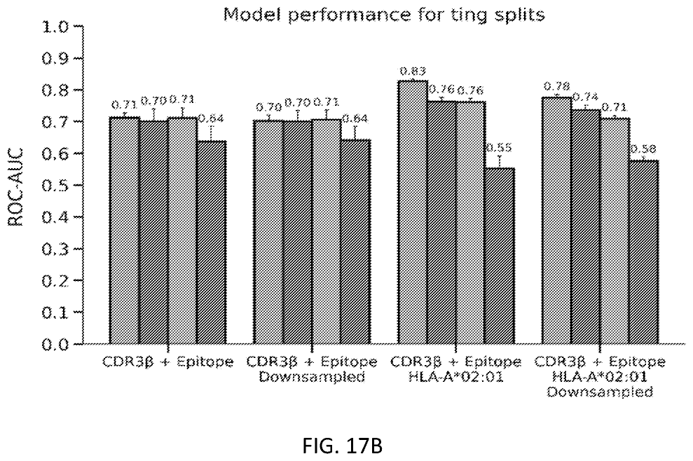

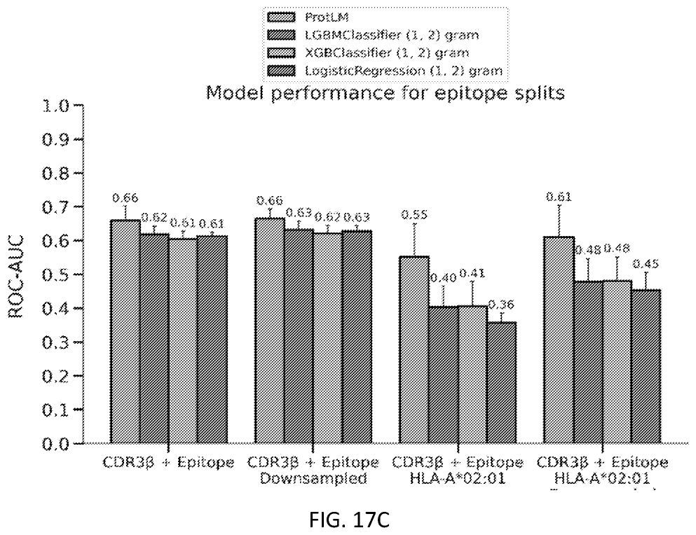

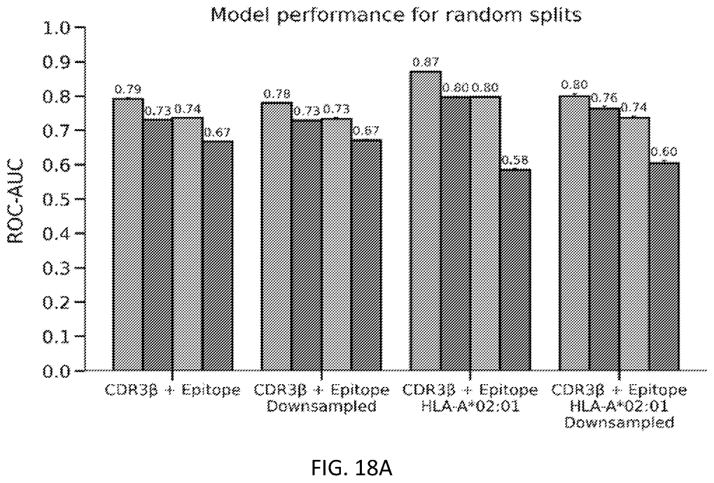

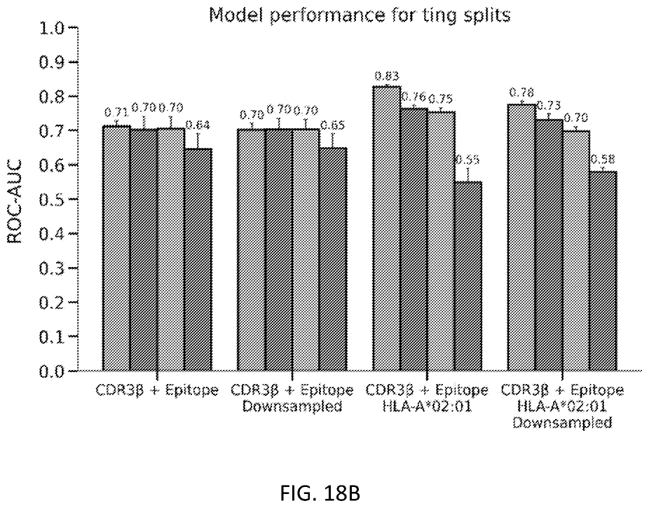

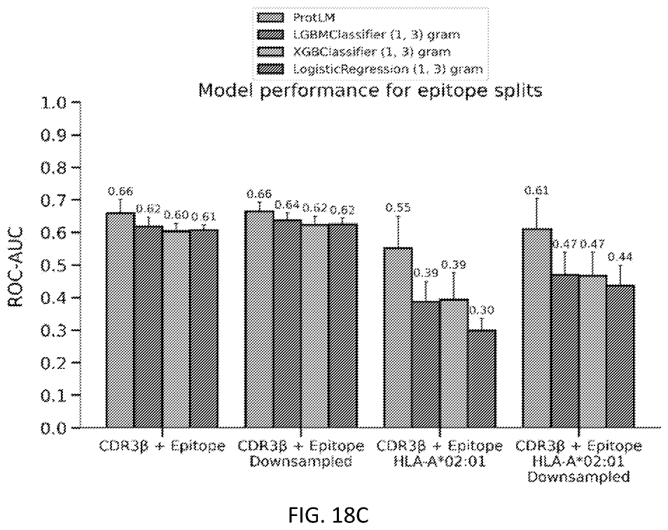

The present approach utilized language modeling to learn the probability distribution of amino acids in each protein sequence. The model was compared with classical n-gram-based machine learning approaches. To accomplish this, TCR sequences ranging from 1-3 grams of words with 1 stride each and fitted logistic regression (LR), extreme gradient boosting (XGB), and light gradient boosting machines (LGBM) models were represented (FIG. 16A-16C). It is important to note that the TCR-based model outperformed n-gram-based machine learning models. This indicated that the present language model captured a significant amount of signal during training on TCRs, which enabled it to outperform models trained exclusively on TCRs. However, it's worth noting that the XGB and LGB models performed similarly to the protein language model in the case of ting-based clusters. These models may be able to recognize motifs from n-gram tokens, which is less the case for logistic regression models.

Benchmarking Against Other Deep Learning-Based Models for TCR-Epitope Binding Prediction

This benchmarking is more focused on addressing embedding, given that the architectures of all three models are based on deep neural networks that have been previously shown to be capable of capturing motifs in the underlying learned tasks.

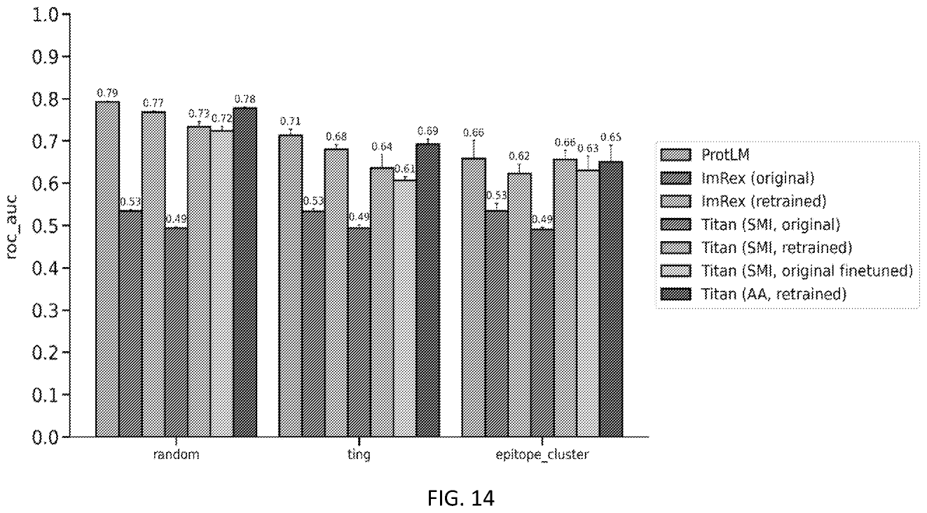

The performance of ImRex and Titan models were examined on the held-out sets used to evaluate protein language NLP model (FIG. 14, ImRex and Titan original models). As shown in FIG. 14, the protein language model outperformed the other two models across all TCR and epitope assignment designs (random: 0.79 vs 0.53 and 0.49; ting: 0.71 vs 0.53 and 0.49; epitope clustering: 0.66 vs 0.53 and 0.49). The Titan model was trained on the entire TCR sequence, whereas the inference in this case was limited to the CDR3 sequences.

To compare the ImRex, TITAN, and protein language NLP model TCR architectures, the two former models were retrained using the same cross-validation sets as for the protein language model. The results summarized in FIG. 14 indicate that retraining Titan and ImRex models enabled them to achieve performance comparable to that of the protein language model. For Titan models, using amino acid encoding for both TCR CDR3 and epitope sequences and letting the model learn their embeddings performed better than using BLOSUM for TCRs and SMILES for epitopes embeddings. Due to the large number of TCRs in the dataset, this approach approximates to language modeling of TCRs.

The fact that the ImRex and protein language models performed similarly demonstrated that the TCR language can indeed capture the physicochemical properties of the amino acids involved in binding between TCR-epitope sequences.

In conclusion, the results demonstrate that TCR-epitope prediction was learnable, that protein language modeling eliminated the need to explicitly feature-engineer the physicochemical properties of amino acids, and that additional data would need to be generated on the TCR and epitope binding to achieve generalization performances greater than 0.66 ROC-AUC.

LIME Framework can be Used to Explain the TCR-Epitope Binding Predictions by the Protein Language Models

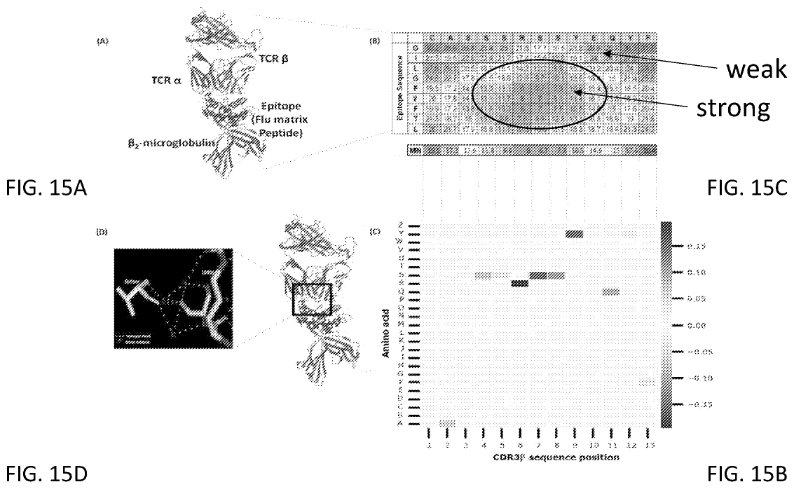

We used the Local Interpretable Model-Agnostic Explanations (LIME) framework to explain TCR-epitope binding predictions made by the protein language model. This approach is illustrated by interpreting the binding predictions using the TCR and epitope sequences in the resolved pHLA-TCR-epitope complex for the PDB entry: 2VLJ³⁵. This complex shows the interaction of CASSRSSSYEQYF CDR3β with its cognate GILGFVFTL epitope as presented by HLA-A*02:01 (FIG. 15A). The pairwise amino acid interactions were calculated between CDR3 β and epitope sequences using this PDB structure in order to highlight the closest distances showing H-bond interactions (FIG. 15B). Interestingly, the importance of Arginine (R) and Serine (S) residues in positions 6 and 7, respectively, in driving the CDR3 β interactions with this epitope from the interpretation of the LIME scores was determined. Additionally, LIME emphasized the significance of serine at positions 4 and 8, as well as tyrosine(Y) and glutamine(Q) at positions 9 and 11, respectively (FIG. 15C). By comparing the LIME heatmap (FIG. 15C) to the distance matrix table (FIG. 15B), we discovered that the highlighted amino-acid positions relevant for the interaction had a high degree of overlap. This was particularly interesting because, despite the fact that the protein model was trained using only linear sequences, identification of specific amino acids (R6 and S7) as contributing to the interaction was possible via LIME analysis (FIG. 15D), which could also be visualized using the PDB 3D interactions.

### Discussion

Accurate predictions of T-cell receptor (TCR)-epitope binding will facilitate the prioritization and rationalization of vaccine antigens, as well as other biomedical applications. The ability to accurately predict TCR-epitope binding could feasibly accelerate the development of new therapeutic and preventative strategies for infectious, autoimmune and chronic diseases. For example, identification of conserved epitopes for SARS-CoV-2 and its corresponding TCRs may allow prioritization of new vaccine candidates². However, developing such models is complicated by the scarcity of data, particularly on the epitope side, and the complexity of the biological systems. Indeed, the diversity of TCRs, HLAs, and their restricted epitopes, combined with cross-reactivity, complicates the task of TCR-binding predictions. The classical immunoinformatic tools do not take T-cell receptors into account and rely heavily on MHC-peptide binding affinity prediction, which does not guarantee that the predicted epitopes are immunogenic (i.e., will bind to TCRs), and thus the achieved performance is prone to high rates of false positives, particularly when testing for immunogenicity^{11,36}. Recently, pan-HLA models have emerged that predict the TCR-epitope bindings directly from their respective amino acid sequences. Among the challenges in developing such models are the following: (i) how to embed amino acids into vectors suitable for training machine/deep learning models while preserving their conservation and physico-chemical properties? (ii) how to create unbiased data sets from published TCR-epitope data sets that account for HLA specificity, cross-reactivity, and a bias toward positive binders? and (iii) how to interpret the models' predictions and identify key amino acid interactors?

The present approach capitalizes on these considerations by developing a protein language model for TCR by using a large corpus of CDR3β sequences in order to comprehend the physicochemical properties of amino acids and their probabilistic co-occurrences, as demonstrated by general protein language models³⁷. Thereafter, through transfer learning of these understandings, the models are fine-tuned to predict the binding between a given epitope and TCR sequence. These models were trained on a TCR-epitope data set prepared from published instances and prepared by balancing negative and positive binders across HLA types.

Training the protein language model on only CDR3β and epitope sequences was sufficient to capture the majority of the prediction signal. When HLA covariates were included in the model, performance was slightly improved. However, when such information was included, the data size decreased, which may have skewed the comparison. It has been predicted that if the model is trained with the same amount of data but with HLA and TCR-alpha information included, performance will improve significantly.

By restricting the model to a single HLA (HLA*A:02), the results improved, indicating that HLA-specific models may be more accurate than pan-HLA models. The fact that this HLA*A:02 has more data, however, may reflect a data-availability bias, which may be addressed by generating additional data for additional HLAs.

The present models were initially trained and tested on random splits; however, equivalent performance was obtained by controlling TCR sequences across train/test data splits in clusters that shared not only similar sequences but also motifs. This generalization to previously unseen TCRs in the last TCR-clustered data splits achieved excellent results, demonstrating that TCR-epitope predictions are learnable and can be used to screen new TCRs for binding to the training set's epitopes. The number of TCR instances per epitope appeared to be irrelevant as long as the TCRs are diverse enough to recognize the epitope-specific motifs driving binding.

Further, by clustering epitope sequences, epitope similarity was controlled between train/test splits. While generalization to previously unknown epitopes performed poorly, the ROC-AUC metrics were significantly greater than random expectation. However, no clear effect of the similarity distance between the epitope sequences in the test and training sets could be established. Taken together, these findings demonstrate that the TCR-epitope binding prediction is both learnable and generalizable to previously unknown epitopes, though the scarcity of epitope sequences in the training data set may act as a constraint on improved generalization.

When compared to state-of-the-art ImRex and Titan models, it was discovered that the present model eliminates the need for feature engineered and BLOSUM embeddings of amino acids to achieve comparable or better results. Additionally, this approach of balancing positive and negative binders across HLA types aided these models in learning more effectively, as evidenced by their improved performance when trained on data splits compared to the inference performance of originally published models. These findings imply that any model capable of learning motifs (i.e., deep learning and nonlinear models) can be used as long as the upstream amino-acid embeddings accurately represent their physicochemical properties and occurrences, in line with observations by Wu et. al.²³.

Finally, the model was perturbed using the Local Interpretable Model-Agnostic Explanations (LIME) technique to gain insight into the mechanism by which the interaction between CDR3 and the epitope sequences was captured. These interpretations were examined further by comparing the putative TCR-epitope interaction sites identified as significant amino acids via LIME to the experimentally determined 3D structures from PDB. It was demonstrated that the present model had learned critical amino acid interactions that are likely to be involved in TCR-epitope binding based on their physical proximity as determined by the 3D structure. While caution should be exercised with all such interpretations, as LIME will provide an interpretation that may or may not agree with the experimental data, this approach provides an intuitive means of quickly verifying the model's predictions.

### CONCLUSION

A novel strategy for predicting and interpreting binding between T-cell receptor and HLA class I epitope using the protein language model for TCRs was developed Amino-acid embeddings that are relevant for this task have been developed. A standard training and evaluation dataset has been generated and the model's performance has been compared to that of classical machine learning models and/or previously published methodologies. By doing this, high accuracy in predicting the binding of previously unseen TCRs and epitopes has been achieved. To aid researchers in deciphering the antigen-specific landscape and underlying immune responses in a variety of disease-related studies, the model's understanding of the interaction between TCR and the relevant epitope sequences using LIME has been examined. The techniques provided herein may be applied to the development of pan-HLA models.

### REFERENCES

1. Morris, G. P. & Allen, P. M. How the TCR balances sensitivity and specificity for the recognition of self and pathogens. Nat. Immunol. 13, 121-128 (2012).
2. Wang, P. et al. Identification of potential vaccine targets for COVID-19 by combining single-cell and bulk TCR sequencing. Clin. Transl. Med. 11, e430-e430 (2021).
3. Rossjohn, J. et al. T cell antigen receptor recognition of antigen-presenting molecules. Annu. Rev. Immunol. 33, 169-200 (2015).
4. La Gruta, N. L., Gras, S., Daley, S. R., Thomas, P. G. & Rossjohn, J. Understanding the drivers of MHC restriction of T cell receptors. Nat. Rev. Immunol. 18, 467-4-78 (2018).
5. Lee, C. H. et al. Predicting Cross-Reactivity and Antigen Specificity of T Cell Receptors. Front. Immunol. 11, 565096 (2020).
6. Dash, P. & Thomas, P. G. The Public Face and Private Lives of T Cell Receptor Repertoires. in Mathematical, Computational and Experimental T Cell Immunology 171-202 (Springer International Publishing, 2021). doi:10.1007/978-3-030-57204-4_11.
7. Ogishi, M. & Yotsuyanagi, H. Quantitative prediction of the landscape of T cell epitope immunogenicity in sequence space. Front. Immunol. 10, 827 (2019).
8. Glanville, J. et al. Identifying specificity groups in the T cell receptor repertoire. Nature 547, 94-98 (2017).
9. Krogsgaard, M. & Davis, M. M. How T cells ‘see’ antigen. Nature Immunology vol. 6 239-245 (2005).
10. Davis, M. M. & Bjorkman, P. J. T-cell antigen receptor genes and T-cell recognition. Nature 334, 395-402 (1988).
11. Peters, B., Nielsen, M. & Sette, A. T cell epitope predictions. Annu. Rev. Immunol. 38, 123-145 (2020).
12. Emerson, R. O. et al. Immunosequencing identifies signatures of cytomegalovirus exposure history and HLA-mediated effects on the T cell repertoire. Nat. Genet. 49, 659-665 (2017).
13. Pogorelyy, M. V et al. Detecting T cell receptors involved in immune responses from single repertoire snapshots. PLOS Biol. 17, 1-13 (2019).
14. Gielis, S. et al. Detection of Enriched T Cell Epitope Specificity in Full T Cell Receptor Sequence Repertoires. Front. Immunol. 10, 2820 (2019).
15. Jokinen, E., Huuhtanen, J., Mustjoki, S., Heinonen, M. & Landesmaki, H. Determining epitope specificity of T cell receptors with TCRGP. bioRxiv 542332 (2019) doi: 10.1101/542332.
16. Jurtz, V. I. et al. NetTCR: sequence-based prediction of TCR binding to peptide-MHC complexes using convolutional neural networks. bioRxiv 433706 (2018).
17. Springer, I., Besser, H., Tickotsky-Moskovitz, N., Dvorkin, S. & Louzoun, Y. Prediction of specific TCR-peptide binding from large dictionaries of TCR-peptide pairs. Front. Immunol. 11, 1803 (2020).
18. Fischer, D. S., Wu, Y., Schubert, B. & Theis, F. J. Predicting antigen specificity of single T cells based on TCR CDR3 regions. Mol. Syst. Biol. 16, e9416 (2020).
19. Moris, P. et al. Current challenges for unseen-epitope TCR interaction prediction and a new perspective derived from image classification. Brief. Bioinform. (2020) doi:10.1093/bib/bbaa318.
20. AlQuraishi, M. The Future of Protein Science will not be Supervised. (2019).
21. Neil, Thomas and Bhattacharya, Nicholas and Rao, R. Can We Learn the Language of Proteins? The Berkeley Artificial Intelligence Research Blog vol. 32 https://bair.berkeley.edu/blog/2019/11/04/proteins/(2019).
22. Rao, R. et al. Evaluating Protein Transfer Learning with TAPE. Adv. Neural Inf. Process. Syst. 32, 9689-9701 (2019).
23. Wu, K. E. et al. TCR-BERT: learning the grammar of T-cell receptors for flexible antigen-binding analyses. bioRxiv 2021.11.18.469186 (2021) doi:10.1101/2021.11.18.469186.
24. Bagaev, D. V et al. VDJdb in 2019: Database extension, new analysis infrastructure and a T-cell receptor motif compendium. Nucleic Acids Res. 48, D1057-D1062 (2020).
25. Vita, R. et al. The immune epitope database (IEDB): 2018 update. Nucleic Acids Res. 47, D339-D343 (2019).
26. Tickotsky, N., Sagiv, T., Prilusky, J., Shifrut, E. & Friedman, N. McPAS-TCR: a manually curated catalogue of pathology-associated T cell receptor sequences. Bioinformatics 33, 2924-2929 (2017).
27. W, Z. et al. PIRD: Pan Immune Repertoire Database. Bioinformatics 36, 897-903 (2020).
28. Mölder, F. et al. Rapid T-cell receptor interaction grouping with ting. Bioinformatics (2021) doi: 10.1093/BIOINFORMATICS/BTAB361.
29. Dhanda, S. K. et al. ImmunomeBrowser: a tool to aggregate and visualize complex and heterogeneous epitopes in reference proteins. Bioinformatics 34, 3931 (2018).
30. Liu, Y. et al. RoBERTa: A Robustly Optimized BERT Pretraining Approach. (2019).
31. Widrich, M. et al. Modern Hopfield Networks and Attention for Immune Repertoire Classification. bioRxiv (2020) doi: 10.1101/2020.04.12.038158.
32. Austel, V. et al. Ribeiro, M. T., Singh, S., Guestrin, C. “Why should I trust you?” Explaining the predictions of any classifier. SIGKDD, 2016. (2017).
33. Berman, H. M. et al. The Protein Data Bank. Nucleic Acids Res. 28, 235-242 (2000).
34. Schrödinger, L. The {PyMOL} Molecular Graphics System, Version 1.8. (2015).
35. Ishizuka, J. et al. The Structural Dynamics and Energetics of an Immunodominant T Cell Receptor Are Programmed by Its Vβ Domain. Immunity 28, 171-182 (2008).
36. Nielsen, M. et al. NetMHCpan, a Method for Quantitative Predictions of Peptide Binding to Any HLA-A and -B Locus Protein of Known Sequence. PLoS One 2, e796 (2007).
37. Strodthoff, N., Wagner, P., Wenzel, M. & Samek, W. UDSMProt: universal deep sequence models for protein classification. Bioinformatics 36, 2401-2409 (2020).

1. A computer-implemented method for training a predictive protein language NLP system to predict binding affinity or a level thereof using natural language processing (NLP) comprising:
in a first phase, training the predictive protein language NLP system comprising a first neural network on TCR sequence datasets and epitope sequence datasets in a self-supervised manner; and
in a second phase, training the predictive protein language NLP system comprising a second neural network with an annotated dataset in a supervised manner to predict binding affinity or a level thereof, wherein the predictive protein language NLP system comprises features from the first phase of training.

2. The computer-implemented method of claim 1 comprising:
in a first phase, training a first transformer of a first neural network with a first dataset comprising TCR sequences in a self-supervised manner;
in a first phase, training a second transformer of a first neural network with a second dataset comprising epitope sequences in a self-supervised manner;
providing the output of the first transformer and second transformer to a cross attention module, wherein the cross attention module computes cross attention using one or more processors between the output of the first transformer model and the output of the second transformer model to improve the prediction of binding affinity.

3. The computer-implemented method of claim 1 comprising:
providing the output of the cross attention module to one or more inputs of a second neural network to determine binding probabilities between tuples, wherein each tuple includes an epitope and a TCR.

4. The computer-implemented method of claim 1, wherein the TCR sequence dataset comprises a plurality of TCR sequences that have each undergone tokenization at an individual amino acid-level, an n-mer level, or a sub-word level.

5. The computer-implemented method of claim 1, wherein the TCR sequence dataset comprises a plurality of TCR sequences that have each undergone tokenization at an individual amino acid-level, an n-mer level, or a sub-word level, and/or wherein the epitope sequence dataset comprises a plurality of epitope sequences that have each undergone tokenization at an individual amino acid-level, an n-mer level, or a sub-word level.

6. The computer-implemented method of claim 1, wherein about 10-20% of the amino acids in the epitope sequence dataset are masked, and/or about 10-20% of the amino acids in the TCR sequence dataset are masked.

7. The computer-implemented method of claim 1, wherein the first transformer model with self-attention further comprises a robustly optimized bidirectional encoder representations from transformers approach model, and the second transformer model with self-attention further comprises a robustly optimized bidirectional encoder representations from transformers approach model.

8. (canceled)

9. The computer-implemented method of claim 1, further comprising:
preprocessing the TCR sequence dataset by selecting for sequences with a specified HLA class;
adding caps at the N-terminus and C-terminus of each TCR sequence if needed;
categorizing the HLA sequences and filtering the dataset based on sequence size; and
clustering the sequences and generating datasets for training.

10. A computer-implemented method for predicting binding affinity or a level thereof between a TCR sequence and an epitope sequence using natural language processing (NLP) comprising:
providing a trained predictive protein language NLP system, wherein:
in a first phase, a predictive protein language NLP system comprising a first neural network is trained on TCR sequence datasets and epitope sequence datasets in a self-supervised manner; and
in a second phase, the predictive protein language NLP system comprising a second neural network is trained using one or more processors, with an annotated protein sequence dataset in a supervised manner to predict binding affinity or a level thereof, wherein the predictive protein language NLP system comprises features from the first phase of training;
receiving an input query, from a user interface device coupled to the trained predictive protein language NLP system, comprising a candidate amino acid sequence;
generating, by the trained predictive protein language NLP system using one or more processors, a prediction including one or more binding affinities or levels thereof for the candidate TCR sequence and epitope sequence; and
displaying, on a display screen of a device, the predicted one or more biophysiochemical properties for the candidate amino acid sequence.

11. (canceled)

12. The computer-implemented method of claim 10, wherein the biophysiochemical property is binding affinity or level thereof of a TCR to an epitope.

13. The computer-implemented method of claim 10, further comprising:
training, in the first phase, the predictive protein language NLP system using the TCR sequence dataset and/or the epitope sequence dataset that has undergone individual amino acid-level tokenization of respective protein sequences.

14. The computer-implemented method of claim 10, further comprising:
training, in the first phase, the predictive protein language NLP system using the TCR sequence dataset and/or the epitope sequence dataset, wherein about 10-20% of the individual amino acids in said datasets are masked.

15. The computer-implemented method of claim 10, wherein the predictive protein language NLP system comprises a salience module, further comprising:
generating, for display on a display screen, information from the salience module that indicates a contribution of respective amino acids to the prediction of the binding affinity of an epitope to a TCR.

16. The computer-implemented method of claim 10, wherein the trained system is compiled into an executable file.

17. The computer-implemented method of claim 10, further comprising:
receiving a plurality of candidate amino acid sequences;
analyzing the candidate amino acid sequences; and
predicting whether the candidate amino acid sequences bind to a TCR epitope.

18. A system or apparatus to predict binding affinity or a level thereof comprising one or more processors for executing instructions corresponding to a predictive protein language NLP system to:
provide a trained predictive protein language NLP system, wherein:
in a first phase, a predictive protein language NLP system comprising a first neural network is trained on TCR sequence datasets and epitope sequence datasets in a self-supervised manner, wherein the first neural network comprises a transformer with attention; and
in a second phase, the predictive protein language NLP system is trained with an annotated sequence dataset in a supervised manner to predict a binding affinity or a level thereof, wherein the predictive protein language NLP system comprises features from the first phase of training;
receive an input query, from a user interface device coupled to the trained predictive protein language NLP system, comprising a candidate amino acid sequence;
generate, by the trained predictive protein language NLP system, a prediction including one or more biophysiochemical properties for the candidate amino acid sequence; and
display, on a display screen of a device, the predicted one or more binding affinity or a level thereof for the candidate TCR sequence and epitope sequence.

19. (canceled)

20. (canceled)

21. The system or apparatus of claim 18, further comprising:
training, in the first phase, the predictive protein language NLP system with a TCR sequence dataset comprising a plurality of TCR sequences that have each undergone tokenization at an individual amino acid-level, an n-mer level, or a sub-word level, and/or with an epitope sequence dataset comprising a plurality of epitope sequences that have each undergone tokenization at an individual amino acid-level, an n-mer level, or a sub-word level.

22. The system or apparatus of claim 18, further comprising:
training, in the first phase, the predictive protein language NLP system, wherein about 10-20% of the amino acids in the epitope sequence dataset are masked, and/or about 10-20% of the amino acids in the TCR sequence dataset are masked.

23. A computer program product for predicting biophysiochemical properties of an amino acid sequence, wherein the computer program product comprises a computer readable storage medium having instructions corresponding to a predictive protein language NLP system embodied therewith, the instructions executable by one or more processors to cause the processors to:
provide a trained predictive protein language NLP system, wherein:
in a first phase, a predictive protein language NLP system comprising a first neural network comprising a first transformer with attention trained on a TCR sequence dataset and a second transformer with attention trained on an epitope sequence dataset in a self-supervised manner; and
in a second phase, the predictive protein language NLP system is trained with an annotated protein sequence dataset in a supervised manner to predict binding affinity or a level thereof, wherein the predictive protein language NLP system comprises features from the first phase of training;
receive an input query, from a user interface device coupled to the trained predictive protein language NLP system, comprising a candidate amino acid sequence;
generate, by the trained predictive protein language NLP system, a prediction including one or more biophysiochemical properties for the candidate amino acid sequence; and
display, on a display screen of a device, the predicted one or more biophysiochemical properties for the candidate amino acid sequence.

24. (canceled)

25. (canceled)

26. The computer program product of claim 23, further comprising:
training, in the first phase, the predictive protein language NLP system using the TCR sequence dataset and/or the epitope sequence dataset that has undergone individual amino acid-level tokenization of respective protein sequences.

27. The computer program product of claim 23, further comprising:
training, in the first phase, the predictive protein language NLP system using the TCR sequence dataset and/or the epitope sequence dataset that has undergone individual amino acid-level tokenization of respective protein sequences.
